SEQUENCE LISTING (175 sequences)

SEQ ID NO: 1 - DNA, 465 bp, Homo sapiens
atggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagc

SEQ ID NO: 2 - protein, 155 aa, Homo sapiens [sequence omitted]

SEQ ID NO: 3 - DNA, 465 bp, Mus musculus
atggctgccagcggcatcacctcgcttcccgcactgccggaggacggcggcgccgccttcccaccaggccacttcaaggaccccaagcggctctactgcaagaacggcggcttcttcctgcgcatccatcccgacggccgcgtggatggcgtccgcgagaagagcgacccacacgtcaaactacaactccaagcagaagagagaggagttgtgtctatcaagggagtgtgtgccaaccggtaccttgctatgaaggaagatggacggctgctggcttctaagtgtgttacagaagagtgtttcttctttgaacgactggaatctaataactacaatacttaccggtcacggaaatactccagttggtatgtggcactgaaacgaactgggcagtataaactcggatccaaaacgggacctggacagaaggccatactgtttcttccaatgtctgctaagagctga

SEQ ID NO: 4 - protein, 154 aa, Mus musculus
MAASGITSLPALPEDGGAAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 5 - DNA, 468 bp, Homo sapiens
atggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaa

SEQ ID NO: 6 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 7 - DNA, 468 bp, Mus musculus
atggctgaaggggagatcacaaccttcgcagccctgaccgagaggttcaacctgcctctaggaaactacaaaaagcccaaactgctctactgcagcaacgggggccacttcttgaggatccttcctgatggcaccgtggatgggacaagggacaggagcgaccagcacattcagctgcagctcagtgcggaaagtgcgggcgaagtgtatataaagggtacggagaccggccagtacttggccatggacaccgaagggcttttatacggctcgcagacaccaaatgaggaatgtctgttcctggaaaggctggaagaaaaccattataacacttacacctccaagaagcatgcggagaagaactggtttgtgggcctcaagaagaacgggagctgtaagcgcggtcctcggactcactatggccagaaagccatcttgtttctgcccctcccggtgtcttctgactag

SEQ ID NO: 8 - protein, 155 aa, Mus musculus
MAEGEITTFAALTERFNLPLGNYKKPKLLYCSNGGHFLRILPDGTVDGTRDRSDQHIQLQLSAESAGEVYIKGTETGQYLAMDTEGLLYGSQTPNEECLFLERLEENHYNTYTSKKHAEKNWFVGLKKNGSCKRGPRTHYGQKAILFLPLPVSSD

SEQ ID NO: 9 - protein, 158 aa, Artificial sequence
  Synthetic polypeptide
MAAGSITTLPALPEDGGSGAFPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 10 - protein, 158 aa, Artificial sequence
  Synthetic polypeptide
MAAGSITTLPALPEDGGSFAFPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 11 - protein, 158 aa, Artificial sequence
  Synthetic polypeptide
MAAGSITTLPALPEDGGSFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 12 - protein, 149 aa, Artificial sequence
  Synthetic polypeptide
PALPEDGGSGAFPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 13 - protein, 150 aa, Artificial sequence
  Synthetic polypeptide
MPALPEDGGSGAFPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 14 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 15 - protein, 131 aa, Artificial sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 16 - protein, 127 aa, Artificial sequence
  Synthetic polypeptide
LYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 17 - protein, 129 aa, Artificial sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 18 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 19 - protein, 140 aa, Artificial sequence
  Synthetic polypeptide
FNLPPGNYKKPVLLYCSNGGHFLRILPDGTVDGTRDRSDQHIQLQVSAESVGEVYIKSTETGQYLAMDTDGLLYGSQTPNEECLFLVRLEENHYVTYISKKHAEKNWFVGLKKNGSCKRGPRTHYGQKAILFLVLPVSSD

SEQ ID NO: 20 - protein, 134 aa, Artificial sequence
  Synthetic polypeptide
NYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCNRGPRTHYGQKAILFLPLPVSSD

SEQ ID NO: 21 - protein, 134 aa, Artificial sequence
  Synthetic polypeptide
NYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCERGPRTHYGQKAILFLPLPVSSD

SEQ ID NO: 22 - protein, 155 aa, Artificial sequence
  Synthetic polypeptide
MAAGSITTLPALPEDGGSFAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 23 - protein, 8 aa, Artificial sequence
  Synthetic polypeptide
AAALPLPV

SEQ ID NO: 24 - protein, 8 aa, Artificial sequence
  Synthetic polypeptide
ILALPLPV

SEQ ID NO: 25 - protein, 8 aa, Artificial sequence
  Synthetic polypeptide
ILFAPLPV

SEQ ID NO: 26 - protein, 8 aa, Artificial sequence
  Synthetic polypeptide
ILFLPAPA

SEQ ID NO: 27 - protein, 131 aa, Artificial sequence; identical to SEQ ID NO: 15
  Synthetic polypeptide

SEQ ID NO: 28 - protein, 146 aa, Artificial sequence
  Synthetic polypeptide
MPALPEDGGAAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 29 - protein, 146 aa, Artificial sequence
  Synthetic polypeptide
MPALPEDGFAAFPPGNYKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 30 - protein, 148 aa, Artificial sequence
  Synthetic polypeptide
MPALPEDGFFSGAFPPGNYKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 31 - DNA, 477 bp, Artificial sequence
  Synthetic polynucleotide
atggccgctggctcaatcacgacgctgccggctctgccggaagatggtggctcaggtgcctttccgccgggtaactacaaaaaaccgaaactgctgtattgcagcaacggcggtcattttctgcgtattctgccggatggcaccgtcgacggtacgcgtgatcgcagtgaccagcacattcagctgcaactgagcgcggaatctgtgggtgaagtttatatcaaatcaaccgaaacgggccagtacctggccatggataccgacggcctgctgtacggttcgcaaacgccgaatgaagaatgcctgtttctggaacgtctggaagaaaaccattacaacacctacatcagtaaaaaacacgcggagaaaaactggttcgttggcctgaagaaaaacggttcctgtaaacgcggcccgcgcacccattacggtcaaaaagccattctgtttctgccgctgccggtttcgtccgactaa

SEQ ID NO: 32 - DNA, 477 bp, Artificial sequence
  Synthetic polynucleotide
atggccgctggctcaatcacgacgctgccggctctgccggaagatggtggctcattcgcctttccgccgggtaactacaaaaaaccgaaactgctgtattgcagcaacggcggtcattttctgcgtattctgccggatggcaccgtcgacggtacgcgtgatcgcagtgaccagcacattcagctgcaactgagcgcggaatctgtgggtgaagtttatatcaaatcaaccgaaacgggccagtacctggccatggataccgacggcctgctgtacggttcgcaaacgccgaatgaagaatgcctgtttctggaacgtctggaagaaaaccattacaacacctacatcagtaaaaaacacgcggagaaaaactggttcgttggcctgaagaaaaacggttcctgtaaacgcggcccgcgcacccattacggtcaaaaagccattctgtttctgccgctgccggtttcgtccgactaa

SEQ ID NO: 33 - DNA, 477 bp, Artificial sequence
  Synthetic polynucleotide
atggccgctggctcaatcacgacgctgccggctctgccggaagatggtggctcattcaacctgccgccgggtaactacaaaaaaccgaaactgctgtattgcagcaacggcggtcattttctgcgtattctgccggatggcaccgtcgacggtacgcgtgatcgcagtgaccagcacattcagctgcaactgagcgcggaatctgtgggtgaagtttatatcaaatcaaccgaaacgggccagtacctggccatggataccgacggcctgctgtacggttcgcaaacgccgaatgaagaatgcctgtttctggaacgtctggaagaaaaccattacaacacctacatcagtaaaaaacacgcggagaaaaactggttcgttggcctgaagaaaaacggttcctgtaaacgcggcccgcgcacccattacggtcaaaaagccattctgtttctgccgctgccggtttcgtccgactaa

SEQ ID NO: 34 - DNA, 468 bp, Artificial sequence
  Synthetic polynucleotide
atggctgaaggcgaaatcacgacgttcaccgctctgacggaaaaattcaatctgccgccgggtaactacaaaaaaccgaaactgctgtattgcagcaacggcggtcattttctgcgtattctgccggatggcaccgtcgacggtacgcgtgatcgcagtgaccagcacattcagctgcaactgagcgcggaatctgtgggtgaagtttatatcaaatcaaccgaaacgggccagtacctggccatggataccgacggcctgctgtacggttcgcaaacgccgaatgaagaatgcctgtttctggaacgtctggaagaaaaccattacaacacctacatcagtaaaaaacacgcggagaaaaactggttcgttggcctgaagaaaaacggttcctgtaaacgcggcccgcgcacgcattacggtcagaaagcaatcctgttcctgccgctgccggtctcctccgactaa

SEQ ID NO: 35 - protein, 155 aa, Artificial sequence; identical to SEQ ID NO: 6
  Synthetic polypeptide

SEQ ID NO: 36 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 37 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLVLPVSSD

SEQ ID NO: 38 - protein, 131 aa, Artificial sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 39 - protein, 129 aa, Artificial sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSVKRGPRTHYGQKAILFLVLPVSSD

SEQ ID NO: 40 - protein, 131 aa, Artificial sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLVLPVSSD

SEQ ID NO: 41 - protein, 129 aa, Artificial sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLVLPVSSD

SEQ ID NO: 42 - protein, 140 aa, Artificial sequence
  Synthetic polypeptide
FNLPPGNYKKPKLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 43 - protein, 140 aa, Artificial sequence
  Synthetic polypeptide
FNLPPGNYKKPKLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 44 - protein, 140 aa, Artificial sequence
  Synthetic polypeptide
FNLPPGNYKKPVLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 45 - protein, 140 aa, Artificial sequence
  Synthetic polypeptide
FNLPPGNYKKPVLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 46 - protein, 131 aa, Artificial sequence
  Synthetic polypeptide
KPKLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 47 - protein, 129 aa, Artificial sequence
  Synthetic polypeptide
KLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 48 - protein, 131 aa, Artificial sequence
  Synthetic polypeptide
KPKLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 49 - protein, 129 aa, Artificial sequence
  Synthetic polypeptide
KLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 50 - protein, 131 aa, Artificial sequence
  Synthetic polypeptide
KPVLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 51 - protein, 129 aa, Artificial sequence
  Synthetic polypeptide
VLLYCSNGGHFLRILPDGTVDGTRDRSDQHIQFQLSAESVGEVYIKSTETGQYLAMDTDGLLYGSQTPNEETLFLERLEENHYNTYISKKHAEKNWFVGLKKNGSVKRGPRTHYGQKAILWLPLPVSSD

SEQ ID NO: 52 - protein, 131 aa, Artificial sequence
  Synthetic polypeptide
KPVLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 53 - protein, 129 aa, Artificial sequence
  Synthetic polypeptide
VLLYCSNGGHFLRILPDGTVDGTRDRSDQHIQFQLSAESVGEVYIKSTETGQYLAMDTDGLLYGSQTPNEETLFLERLEENHYVTYISKKHAEKNWFVGLKKNGSVKRGPRTHYGQKAILWLPLPVSSD

SEQ ID NO: 54 - protein, 134 aa, Artificial sequence
  Synthetic polypeptide
FNLPPGNYKKPKLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 55 - protein, 134 aa, Artificial sequence
  Synthetic polypeptide
FNLPPGNYKKPVLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 56 - protein, 134 aa, Artificial sequence
  Synthetic polypeptide
FNLPPGNYKKPVLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 57 - protein, 134 aa, Artificial sequence; identical to SEQ ID NO: 56
  Synthetic polypeptide

SEQ ID NO: 58 - protein, 125 aa, Artificial sequence
  Synthetic polypeptide
KPKLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 59 - protein, 123 aa, Artificial sequence
  Synthetic polypeptide
KLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 60 - protein, 125 aa, Artificial sequence
  Synthetic polypeptide
KPVLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 61 - protein, 123 aa, Artificial sequence
  Synthetic polypeptide
VLLYCSNGGHFLRILPDGTVDGTRDRSDQHIQFQLSAESVGEVYIKSTETGQYLAIDTDGLVYGSQTPNEECLFLERLEENHYNTYISKKHGWFLGIKKNGSVKGTHYGQKAILFLPLPVSSD

SEQ ID NO: 62 - protein, 125 aa, Artificial sequence
  Synthetic polypeptide
KPKLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 63 - protein, 123 aa, Artificial sequence
  Synthetic polypeptide
KLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 64 - protein, 125 aa, Artificial sequence
  Synthetic polypeptide
VPKLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 65 - protein, 123 aa, Artificial sequence
  Synthetic polypeptide
VLLYCSNGGHFLRILPDGTVDGTRDRSDQHIQFQLSAESVGEVYIKSTETGQYLAIDTDGLVYGSQTPNEECLFLERLEENHYVTYISKKHGWFLGIKKNGSVKGTHYGQKAILFLPLPVSSD

SEQ ID NO: 66 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCNRGPRTHYGQKAILFLPLPVSSD

SEQ ID NO: 67 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCERGPRTHYGQKAILFLPLPVSSD

SEQ ID NO: 68 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 69 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVNRGPRTHYGQKAILFLPLPVSSD

SEQ ID NO: 70 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVERGPRTHYGQKAILFLPLPVSSD

SEQ ID NO: 71 - protein, 131 aa, Artificial sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 72 - protein, 129 aa, Artificial sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 73 - protein, 131 aa, Artificial sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 74 - protein, 129 aa, Artificial sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 75 - protein, 131 aa, Artificial sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 76 - protein, 129 aa, Artificial sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 77 - protein, 131 aa, Artificial sequence
  Synthetic polypeptide
KPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCNRGPRTHYGQKAILFLPLPVSSD

SEQ ID NO: 78 - protein, 129 aa, Artificial sequence
  Synthetic polypeptide
VLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCERGPRTHYGQKAILFLPLPVSSD

SEQ ID NO: 79 - protein, 131 aa, Artificial sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCNRGPRTHYGQKAILFLPLPVSSD

SEQ ID NO: 80 - protein, 129 aa, Artificial sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCERGPRTHYGQKAILFLPLPVSSD

SEQ ID NO: 81 - protein, 140 aa, Artificial sequence
  Synthetic polypeptide
FNLPPGNYTTPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 82 - protein, 140 aa, Artificial sequence
  Synthetic polypeptide
FNLPPGNYTTPKLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 83 - protein, 140 aa, Artificial sequence
  Synthetic polypeptide
FNLPPGNYTTPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCNRGPRTHYGQKAILFLPLPVSSD

SEQ ID NO: 84 - protein, 140 aa, Artificial sequence
  Synthetic polypeptide
FNLPPGDQDQNQLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 85 - protein, 131 aa, Artificial sequence
  Synthetic polypeptide
KPKLLYCSNGGHFLRILPDGTVDGTRDRSDPHIQLQLIAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 86 - protein, 129 aa, Artificial sequence
  Synthetic polypeptide
KLLYCSNGGHFLRILPDGTVDGTRDRSDPHIQLQLIAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 87 - protein, 140 aa, Artificial sequence
  Synthetic polypeptide
FNLPPGNYKKPVLLYCSNGGHFLRILPDGTVDGTRDRSDPHIQLQLIAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 88 - protein, 131 aa, Artificial sequence
  Synthetic polypeptide
KPVLLYCSNGGHFLRILPDGTVDGTRDRSDPHIQLQLIAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 89 - protein, 129 aa, Artificial sequence
  Synthetic polypeptide
VLLYCSNGGHFLRILPDGTVDGTRDRSDPHIQLQLIAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 90 - protein, 131 aa, Artificial sequence
  Synthetic polypeptide
KPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 91 - protein, 129 aa, Artificial sequence
  Synthetic polypeptide
KLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 92 - protein, 140 aa, Artificial sequence
  Synthetic polypeptide
FNLPPGNYKKPVLLYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 93 - protein, 131 aa, Artificial sequence
  Synthetic polypeptide
KPVLLYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 94 - protein, 129 aa, Artificial sequence
  Synthetic polypeptide
VLLYCSNGGHFLRILPDGTVDGTRDRSDPHIQLQLIAESVGEVYIKSTETGQYLAMDTDGLLYGSQTPNEECLFLERLEENGYVTYISKKHAEKNWFVGLKKNGSCKRGPRTHYGQKAILFLPLPVSSD

SEQ ID NO: 95 - protein, 131 aa, Artificial sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSPVRGPRTHYGQKAILFLPLPVSSD

SEQ ID NO: 96 - protein, 129 aa, Artificial sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSPVRGPRTHYGQKAILFLPLPVSSD

SEQ ID NO: 97 - protein, 140 aa, Artificial sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSPVRGPRTHYGQKAILFLPLPVSSD

SEQ ID NO: 98 - protein, 140 aa, Artificial sequence
  Synthetic polypeptide
FNLPPGNYKKPKLLYCSNGGHFLRILPDGTVDGTEDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 99 - protein, 158 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCKRGPRTHYGQKAILFLPLPVSSD

SEQ ID NO: 100 - protein, 158 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCNRGPRTHYGQKAILFLPLPVSSD

SEQ ID NO: 101 - protein, 158 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSCERGPRTHYGQKAILFLPLPVSSD

SEQ ID NO: 102 - protein, 158 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 103 - protein, 158 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVNRGPRTHYGQKAILFLPLPVSSD

SEQ ID NO: 104 - protein, 158 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVERGPRTHYGQKAILFLPLPVSSD

SEQ ID NO: 105 - protein, 90 aa, Artificial sequence
  Synthetic polypeptide
CGADQFRCGNGSCVPRAWRCDGVDDCGDGSDEAPEICETPTCQSNEFRCRSGRCIPQHWLCDGLNDCGDGSDESQQCSAPASEPPGSLSL

SEQ ID NO: 106 - protein, 37 aa, Artificial sequence
  Synthetic polypeptide
CGADQFRCGNGSCVPRAWRCDGVDDCGDGSDEAPEIC

SEQ ID NO: 107 - protein, 37 aa, Artificial sequence
  Synthetic polypeptide
CQSNEFRCRSGRCIPQHWLCDGLNDCGDGSDEXXXXC

SEQ ID NO: 108 - protein, 38 aa, Artificial sequence
  Synthetic polypeptide
CXXXXXXXCXXGXCXPXXWXCDGXXDCXDXSDEXXXXC

SEQ ID NO: 109 - protein, 38 aa, Artificial sequence
  Synthetic polypeptide
CXXXXFXCXXXXXCXPXXWXCDGXXDCGDXSDEXXXXC

SEQ ID NO: 110 - protein, 47 aa, Artificial sequence
  Synthetic polypeptide
CQSNEFRCRSGRCIPQHWLCDGLNDCGDGSDESQQCSAPASEPPGSL

SEQ ID NO: 111 - protein, 36 aa, Artificial sequence
  Synthetic polypeptide
CQSNEFRCRSGRCIPQHWLCDGLNDCGDGSDESQQC

SEQ ID NO: 112 - protein, 35 aa, Artificial sequence
  Synthetic polypeptide
CRAGEFRCSNGRCVPLTWLCDGEDDCQDNSDEKNC

SEQ ID NO: 113 - protein, 38 aa, Artificial sequence
  Synthetic polypeptide
CPSNQFPCRSTGICIPLAWVCDGLNDCGDGSDESPAHC

SEQ ID NO: 114 - protein, 37 aa, Artificial sequence
  Synthetic polypeptide
CQSNEFRCRSGRCIPQHWLCDGLNDCGDGSDEPPAHC

SEQ ID NO: 115 - protein, 37 aa, Artificial sequence
  Synthetic polypeptide
CLPDEFQCXSGRCIPQXWXCDGLNDCGDGSDEXPAXC

SEQ ID NO: 116 - protein, 38 aa, Artificial sequence
  Synthetic polypeptide
CXXXXFXCXNXXXCXXXXWXCDGXDDCXDGSDEXXXXC

SEQ ID NO: 117 - protein, 37 aa, Artificial sequence
  Synthetic polypeptide
CGPDQFRCSSGKCIPQHWLCDGLNDCGDGSDESPATC

SEQ ID NO: 118 - protein, 37 aa, Artificial sequence
  Synthetic polypeptide
CGANEFQCRSTGICVPVEWVCDGDNDCGDGSDEPPVC

SEQ ID NO: 119 - protein, 37 aa, Artificial sequence; identical to SEQ ID NO: 106
  Synthetic polypeptide

SEQ ID NO: 120 - protein, 37 aa, Artificial sequence
  Synthetic polypeptide
CGPDEFRCNNGQCIPLPWRCDGVDDCGDNSDEPLEIC

SEQ ID NO: 121 - protein, 35 aa, Artificial sequence
  Synthetic polypeptide
CASGEFTCNNGQCVPLAWRCDGVNDCQDGSDEKGC

SEQ ID NO: 122 - protein, 36 aa, Artificial sequence
  Synthetic polypeptide
CPPDEFQCRGTKKCLPLAWVCDGDNDCEDDSDEESC

SEQ ID NO: 123 - protein, 37 aa, Artificial sequence
  Synthetic polypeptide
CLPDEFQCGSGRCIPQHWLCDGLNDCGDGSDEPPAHC

SEQ ID NO: 124 - protein, 36 aa, Artificial sequence
  Synthetic polypeptide
CAPGEFTCKNTGRCIPLNWRCDGDDDCGDGSDETDC

SEQ ID NO: 125 - protein, 29 aa, Artificial sequence
  Synthetic polypeptide
CXXXXFXCXXXXXCXPXXWXCDGXXDCGD

SEQ ID NO: 126 - protein, 48 aa, Artificial sequence
  Synthetic polypeptide
CGADQFRCGNGSCVPRAWRCDGVDDCGDGSDEAPEICETPTCQSNEFR

SEQ ID NO: 127 - protein, 40 aa, Artificial sequence
  Synthetic polypeptide
CRSGRCIPQHWLCDGLNDCGDGSDESQQCSAPASEPPGSL

SEQ ID NO: 128 - protein, 38 aa, Artificial sequence; identical to SEQ ID NO: 116
  Synthetic polypeptide

SEQ ID NO: 129 - protein, 38 aa, Artificial sequence; identical to SEQ ID NO: 108
  Synthetic polypeptide

SEQ ID NO: 130 - protein, 38 aa, Artificial sequence; identical to SEQ ID NO: 109
  Synthetic polypeptide

SEQ ID NO: 131 - protein, 36 aa, Artificial sequence
  Synthetic polypeptide
CGXXLFTCXXXXICISXXWXCDGXDDCXDNSDEXXC

SEQ ID NO: 132 - protein, 47 aa, Artificial sequence
  Synthetic polypeptide
CGEGLFTCRSTNICISHAWVCDGVDDCEDNSDENNCSAPASEPPGSL

SEQ ID NO: 133 - protein, 36 aa, Artificial sequence
  Synthetic polypeptide
CGAGLFTCRSTNICISQVWVCDGVDDCEDNSDEDSC

SEQ ID NO: 134 - protein, 36 aa, Artificial sequence
  Synthetic polypeptide
CGAGLFTCRSTNICISQAWVCDGVDDCEDNSDENYC

SEQ ID NO: 135 - protein, 36 aa, Artificial sequence
  Synthetic polypeptide
CGAGLFTCRSTNICISQAWVCDGVDDCEDNSDETNC

SEQ ID NO: 136 - protein, 36 aa, Artificial sequence
  Synthetic polypeptide
CGEGLFTCGSTNICISSAWVCDGVDDCEDNSDENNC

SEQ ID NO: 137 - protein, 36 aa, Artificial sequence
  Synthetic polypeptide
CGEGLFTCRSTNICISHAWVCDGVDDCEDNSDENNC

SEQ ID NO: 138 - protein, 36 aa, Artificial sequence
  Synthetic polypeptide
CGEGLFTCRSTNICISEAWICDGVDDCEDNSDEKNC

SEQ ID NO: 139 - protein, 36 aa, Artificial sequence
  Synthetic polypeptide
CGAGLFTCRSAKICISHAWVCDGIDDCEDNSDENNC

SEQ ID NO: 140 - protein, 36 aa, Artificial sequence
  Synthetic polypeptide
CGAGLFTCRNSKICISQAWVCDGVDDCDDNSDEKYC

SEQ ID NO: 141 - protein, 36 aa, Artificial sequence
  Synthetic polypeptide
CGASLFTCRRSNICISQAWVCDGVDDCEDNSDEMNC

SEQ ID NO: 142 - protein, 36 aa, Artificial sequence
  Synthetic polypeptide
CGAGLFTCRSTKICISQAWVCDGVDDCEDNSDEKNC

SEQ ID NO: 143 - protein, 47 aa, Artificial sequence
  Synthetic polypeptide
CGAGLFTCRSTNICISQVWVCDGVDDCEDNSDEDSCSAPASEPPGSL

SEQ ID NO: 144 - protein, 47 aa, Artificial sequence
  Synthetic polypeptide
CGAGLFTCRSTNICISQAWVCDGVDDCEDNSDENYCSAPASEPPGSL

SEQ ID NO: 145 - protein, 47 aa, Artificial sequence
  Synthetic polypeptide
CGAGLFTCRSTNICISQAWVCDGVDDCEDNSDETNCSAPASEPPGSL

SEQ ID NO: 146 - protein, 47 aa, Artificial sequence
  Synthetic polypeptide
CGEGLFTCGSTNICISSAWVCDGVDDCEDNSDENNCSAPASEPPGSL

SEQ ID NO: 147 - protein, 47 aa, Artificial sequence
  Synthetic polypeptide
CGEGLFTCRSTNICISEAWICDGVDDCEDNSDEKNCSAPASEPPGSL

SEQ ID NO: 148 - protein, 47 aa, Artificial sequence
  Synthetic polypeptide
CGAGLFTCRSAKICISHAWVCDGIDDCEDNSDENNCSAPASEPPGSL

SEQ ID NO: 149 - protein, 47 aa, Artificial sequence
  Synthetic polypeptide
CGAGLFTCRNSKICISQAWVCDGVDDCDDNSDEKYCSAPASEPPGSL

SEQ ID NO: 150 - protein, 47 aa, Artificial sequence
  Synthetic polypeptide
CGASLFTCRRSNICISQAWVCDGVDDCEDNSDEMNCSAPASEPPGSL

SEQ ID NO: 151 - protein, 47 aa, Artificial sequence
  Synthetic polypeptide
CGAGLFTCRSTKICISQAWVCDGVDDCEDNSDEKNCSAPASEPPGSL

SEQ ID NO: 152 - protein, 135 aa, Artificial sequence
  Synthetic polypeptide
CGADQFRCGNGSCVPRAWRCDGVDDCGDGSDEAPEICETPTCQSNEFRCRSGRCIPQHWLCDGLNDCGDGSDESQQCSAPASEPPGSLCGEGLFTCRSTNICISHAWVCDGVDDCEDNSDENNCSAPASEPPGSL

SEQ ID NO: 153 - protein, 134 aa, Artificial sequence
  Synthetic polypeptide
CLPDEFQCGSGRCIPQHWLCDGLNDCGDGSDEPPAHCSAPASEPPGSLCRAGEFRCSNGRCVPLTWLCDGEDDCQDNSDEKNCAQPTCGEGLFTCRSTNICISHAWVCDGVDDCEDNSDENNCSAPASEPPGSL

SEQ ID NO: 154 - protein, 136 aa, Artificial sequence
  Synthetic polypeptide
CAPGEFTCKNTGRCIPLNWRCDGDDDCGDGSDETDCPAPTCPSNQFPCRSTGICIPLAWVCDGLNDCGDGSDESPAHCSAPASEPPGSLCGEGLFTCRSTNICISHAWVCDGVDDCEDNSDENNCSAPASEPPGSL

SEQ ID NO: 155 - protein, 136 aa, Artificial sequence
  Synthetic polypeptide
CGPDEFRCNNGQCIPLPWRCDGVDDCGDNSDEPLEICQAPTCQSNEFRCRSGRCIPQHWLCDGLNDCGDGSDEPPAHCSAPASEPPGSLCGEGLFTCRSTNICISHAWVCDGVDDCEDNSDENNCSAPASEPPGSL

SEQ ID NO: 156 - protein, 248 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSDCGADQFRCGNGSCVPRAWRCDGVDDCGDGSDEAPEICETPTCQSNEFRCRSGRCIPQHWLCDGLNDCGDGSDESQQCSAPASEPPGSLSL

SEQ ID NO: 157 - protein, 338 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSDCGADQFRCGNGSCVPRAWRCDGVDDCGDGSDEAPEICETPTCQSNEFRCRSGRCIPQHWLCDGLNDCGDGSDESQQCSAPASEPPGSLSLCGADQFRCGNGSCVPRAWRCDGVDDCGDGSDEAPEICETPTCQSNEFRCRSGRCIPQHWLCDGLNDCGDGSDESQQCSAPASEPPGSLSL

SEQ ID NO: 158 - protein, 205 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSDCGEGLFTCRSTNICISHAWVCDGVDDCEDNSDENNCSAPASEPPGSL

SEQ ID NO: 159 - protein, 252 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSDCGEGLFTCRSTNICISHAWVCDGVDDCEDNSDENNCSAPASEPPGSLCGEGLFTCRSTNICISHAWVCDGVDDCEDNSDENNCSAPASEPPGSL

SEQ ID NO: 160 - protein, 295 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSDCGADQFRCGNGSCVPRAWRCDGVDDCGDGSDEAPEICETPTCQSNEFRCRSGRCIPQHWLCDGLNDCGDGSDESQQCSAPASEPPGSLSLCGEGLFTCRSTNICISHAWVCDGVDDCEDNSDENNCSAPASEPPGSL

SEQ ID NO: 161 - protein, 295 aa, Artificial sequence
  Synthetic polypeptide
MAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSDCGEGLFTCRSTNICISHAWVCDGVDDCEDNSDENNCSAPASEPPGSLCGADQFRCGNGSCVPRAWRCDGVDDCGDGSDEAPEICETPTCQSNEFRCRSGRCIPQHWLCDGLNDCGDGSDESQQCSAPASEPPGSLSL

SEQ ID NO: 162 - protein, 248 aa, Artificial sequence
  Synthetic polypeptide
CGADQFRCGNGSCVPRAWRCDGVDDCGDGSDEAPEICETPTCQSNEFRCRSGRCIPQHWLCDGLNDCGDGSDESQQCSAPASEPPGSLSLMAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 163 - protein, 338 aa, Artificial sequence
  Synthetic polypeptide
CGADQFRCGNGSCVPRAWRCDGVDDCGDGSDEAPEICETPTCQSNEFRCRSGRCIPQHWLCDGLNDCGDGSDESQQCSAPASEPPGSLSLCGADQFRCGNGSCVPRAWRCDGVDDCGDGSDEAPEICETPTCQSNEFRCRSGRCIPQHWLCDGLNDCGDGSDESQQCSAPASEPPGSLSLMAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 164 - protein, 205 aa, Artificial sequence
  Synthetic polypeptide
CGEGLFTCRSTNICISHAWVCDGVDDCEDNSDENNCSAPASEPPGSLMAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 165 - protein, 252 aa, Artificial sequence
  Synthetic polypeptide
CGEGLFTCRSTNICISHAWVCDGVDDCEDNSDENNCSAPASEPPGSLCGEGLFTCRSTNICISHAWVCDGVDDCEDNSDENNCSAPASEPPGSLMAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 166 - protein, 295 aa, Artificial sequence
  Synthetic polypeptide
CGEGLFTCRSTNICISHAWVCDGVDDCEDNSDENNCSAPASEPPGSLCGADQFRCGNGSCVPRAWRCDGVDDCGDGSDEAPEICETPTCQSNEFRCRSGRCIPQHWLCDGLNDCGDGSDESQQCSAPASEPPGSLSLMAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 167 - protein, 295 aa, Artificial sequence
  Synthetic polypeptide
CGADQFRCGNGSCVPRAWRCDGVDDCGDGSDEAPEICETPTCQSNEFRCRSGRCIPQHWLCDGLNDCGDGSDESQQCSAPASEPPGSLSLCGEGLFTCRSTNICISHAWVCDGVDDCEDNSDENNCSAPASEPPGSLMAAGSITTLPALPEDGGSGAFPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLKKNGSVKRGPRTHYGQKAILFLPLPVSSD

SEQ ID NO: 168 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSCVRGPRTHYGQKAILFLPLPVSSD

SEQ ID NO: 169 - protein, 140 aa, Artificial Sequence
  Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSVVRGPRTHYGQKAILFLPLPVSSD

SEQ ID NO: 170 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSCVRGPRTHYGQKAILFLPLPVSSD

SEQ ID NO: 171 - protein, 129 aa, Artificial Sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSCVRGPRTHYGQKAILFLPLPVSSD

SEQ ID NO: 172 - protein, 131 aa, Artificial Sequence
  Synthetic polypeptide
KPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSVVRGPRTHYGQKAILFLPLPVSSD

SEQ ID NO: 173 - protein, 129 aa, Artificial Sequence
  Synthetic polypeptide
KLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLDQNGSVVRGPRTHYGQKAILFLPLPVSSD

SEQ ID NO: 174 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLDQNGSCVRGPRTHYGQKAILFLPLPVSSD

SEQ ID NO: 175 - protein, 140 aa, Artificial Sequence
  Synthetic polypeptide
FNLPPGNYKKPVLLYCSNGGHFLRILPDGTVDGTRDRSDQHIQLQLSAESVGEVYIKSTETGQYLAMDTDGLLYGSQTPNEECLFLERLEENHYVTYISKKHAEKNWFVGLDQNGSVVRGPRTHYGQKAILFLPLPVSSD

(19) United States
(12) Patent Application Publication
(10) Pub. No.: US 2016/0237133 A1
(43) Pub. Date: Aug. 18, 2016

(54) CHIMERIC FIBROBLAST GROWTH FACTOR (FGF) 2/FGF1 PEPTIDES AND METHODS OF USE

(71) Applicant: Salk Institute for Biological Studies, La Jolla, CA (US)

(72) Inventors: Jae Myoung Suh, San Diego, CA (US); Michael Downes, San Diego, CA (US); Ronald M. Evans, La Jolla, CA (US); Annette Atkins, San Diego, CA (US)

(73) Assignee: Salk Institute for Biological Studies, La Jolla, CA (US)

(21) Appl. No.: 15/092,097

(22) Filed: Apr. 6, 2016

Related U.S. Application Data
(63) Continuation of application No. PCT/US2014/061624, filed on Oct. 21, 2014.
(60) Provisional application No. 61/893,775, filed on Oct. 21, 2013.
(60) Provisional application No. 61/949,962, filed on Mar. 7, 2014.
(60) Provisional application No. 62/018,758, filed on Jun. 30, 2014.

(51) Int. Cl. C07K 14/50; A61K 45/06; A61K 38/18

(52) U.S. Cl. CPC C07K 14/503; C07K 14/501; A61K 38/1825; A61K 45/06; C07K 2319/00

(57) ABSTRACT

The present disclosure provides chimeric proteins having an N-terminus coupled to a C-terminus, wherein the N-terminus comprises an N-terminal portion of fibroblast growth factor (FGF) 2 and the C-terminus comprises a portion of an FGF1 protein. Such FGF2/FGF1 chimeras can further include a fibroblast growth factor receptor (FGFR) 1c-binding protein, a β-Klotho-binding protein, or both. Also provided are nucleic acid molecules that encode such proteins, and vectors and cells that include such nucleic acids. Methods of using the disclosed molecules to reduce blood glucose levels are also provided.

### CROSS-REFERENCE TO RELATED APPLICATIONS

This is a continuation of International Application No. PCT/US2014/061624 filed Oct. 21, 2014, which was published in English under PCT Article 21(2), which in turn claims priority to U.S. Provisional Application No. 61/893,775 filed Oct. 21, 2013, U.S. Provisional Application No. 61/949,962 filed Mar. 7, 2014, and U.S. Provisional Application No. 62/018,758 filed Jun. 30, 2014, all herein incorporated by reference.

### ACKNOWLEDGEMENT OF GOVERNMENT SUPPORT

This invention was made with government support under Grant Nos. DK057978, DK090962, HL088093, HL105278 and ES010337 awarded by The National Institutes of Health, National Human Genome Research Institute. The government has certain rights in the invention.

### FIELD

This application provides FGF2/FGF1 chimeric proteins, nucleic acids encoding such proteins, and methods of their use, for example to treat a metabolic disease.

### BACKGROUND

Type 2 diabetes and obesity are leading causes of mortality and are associated with the Western lifestyle, which is characterized by excessive nutritional intake and lack of exercise. A central player in the pathophysiology of these diseases is the nuclear hormone receptor (NHR) PPARγ, a lipid sensor and master regulator of adipogenesis. PPARγ is also the molecular target for the thiazolidinedione (TZD)-class of insulin sensitizers, which command a large share of the current oral anti-diabetic drug market. However, there are numerous side effects associated with the use of TZDs such as weight gain, liver toxicity, upper respiratory tract infection, headache, back pain, hyperglycemia, fatigue, sinusitis, diarrhea, hypoglycemia, mild to moderate edema, and anemia. Thus, the identification of new insulin sensitizers is needed.

### SUMMARY

Provided herein are chimeric proteins that include an N-terminus coupled to a C-terminus, wherein the N-terminus comprises an N-terminal portion of fibroblast growth factor (FGF) 2 and the C-terminus comprises a portion of an FGF1 protein. In some examples, the N-terminus includes at least 12 consecutive amino acids from amino acids 1-30 of FGF2 (e.g., from SEQ ID NO: 2 or 4) (which in some examples can include at least one mutation, such as 1, 2, 3 or 4 point mutations, such as substitutions, deletions, or additions, such as addition of a methionine on the N-terminus and/or such as those shown in Table 3) and the C-terminus includes at least 120 consecutive amino acids from amino acids 5-141 of FGF1 (e.g., from SEQ ID NO: 6 or 8), (which in some examples can include at least one mutation, such as 1 to 3, 1 to 4, 1 to 5, 1 to 10 or 1 to 20 point mutations, such as substitutions, deletions, additions, or combinations thereof, such as those shown in Table 2). Specific exemplary FGF2/FGF1 chimeric proteins are shown in SEQ ID NOS: 9, 10, 11, 12, 13, 99, 100, 101, 102, 103 and 104.

In some examples, the FGF2/FGF1 chimera includes additional sequences at its N- and/or C-terminus (e.g., see FIGS. 19, 20, 21 and 22). In one example the FGF2/FGF1 chimera includes at its N- and/or C-terminus at least 10, at least 20, at least 30, at least 35, at least 40, at least 50, at least 60, at least 70, at least 80, at least 90, at least 100, at least 120, at least 150, at least 180, or at least 200 amino acids (such as 20-500, 20 to 250, 30 to 200, 35 to 180, 37 to 90, or 37 to 180 amino acids) of a protein that selectively binds to beta-Klotho (β-Klotho), such as any of SEQ ID NOS: 105, 106, 107, 108, 109, 110, 111, 112, 113, 114, 115, 116, 117, 118, 119, 120, 121, 122, 123, 124, 125, 126, 127, 1289, 129, or 130.

In some examples, the FGF2/FGF1 chimera includes additional sequences at its N- and/or C-terminus (e.g., see FIGS. 19-22). In one example the FGF2/FGF1 chimera includes at least 10, at least 20, at least 30, at least 35, at least 40, at least 50, at least 60, at least 70, at least 80, at least 90, at least 100, at least 120, at least 150, at least 180, or at least 200 amino acids (such as 20-500, 20 to 250, 30 to 200, 35 to 180, 37 to 90, or 37 to 180 amino acids) of a protein that selectively binds to FGFR1c, such as any of SEQ ID NOS: 131, 132, 133, 134, 135, 136, 137, 138, 139, 140, 141, 142, 143, 144, 145, 146, 147, 148, 149, 150 and 151.

In some examples, the FGF2/FGF1 chimera includes additional sequences at its N- and/or C-terminus (e.g., see FIGS. 19-22). In one example the FGF2/FGF1 chimera includes at least 10, at least 20, at least 30, at least 35, at least 40, at least 50, at least 60, at least 70, at least 80, at least 90, at least 100, at least 120, at least 150, at least 180, or at least 200 amino acids (such as 20-500, 20 to 250, 30 to 200, 35 to 180, 37 to 90, or 37 to 180 amino acids) of a protein that selectively binds to β-Klotho and/or includes at least 10, at least 20, at least 30, at least 35, at least 40, at least 50, at least 60, at least 70, at least 80, at least 90, at least 100, at least 120, at least 150, at least 180, or at least 200 amino acids (such as 20-500, 20 to 250, 30 to 200, 35 to 180, 37 to 90, or 37 to 180 amino acids) of a protein that selectively binds to FGFR1c, such as any of SEQ ID NOS: 160, 161, 162, 163, 164, 165, 166, or 167.

Also provided are isolated nucleic acid molecules encoding the disclosed FGF2/FGF1 chimeric proteins. Vectors and cells that include such nucleic acid molecules are also provided.

Methods of using the disclosed FGF2/FGF1 chimeric proteins (or nucleic acid molecules encoding such) are provided. In some examples the methods include administering a therapeutically effective amount of a disclosed FGF2/FGF1 chimeric protein (or nucleic acid molecules encoding such) to reduce blood glucose in a mammal, such as a decrease of at least 5%, at least 10%, or at least 20%, for example relative to an amount when no chimera is administered. In some examples the methods include administering a therapeutically effective amount of a disclosed FGF2/FGF1 chimeric protein (or nucleic acid molecules encoding such) to treat a metabolic disease in a mammal, such as diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular diseases (e.g., hypertension), or combinations thereof. Also provided are methods of reducing fed and fasting blood glucose, improving insulin sensitivity and glucose tolerance, reducing systemic chronic inflammation, ameliorating hepatic steatosis in a mammal, or combinations thereof, by administering a therapeutically effective amount of a disclosed FGF2/FGF1 chimeric protein (or nucleic acid molecules encoding such). In some examples, use of the disclosed FGF2/FGF1 chimeric proteins (or nucleic acid molecules encoding such) results in one or more of: reduction in triglycerides, decrease in insulin resistance, reduction of hyperinsulinemia, increase in glucose tolerance, reduction of hyperglycemia, or combinations thereof, in a mammal.

The foregoing and other objects and features of the disclosure will become more apparent from the following detailed description, which proceeds with reference to the accompanying figures.

### BRIEF DESCRIPTION OF THE DRAWINGS

FIG. 1 is a graph showing decreases in blood glucose following administration of various peptides (mouse FGF1; positive control (SEQ ID NO: 8 but lacking first 15 as MAEGEITTFAALTER and with an added M at N terminus); FGF24 (SEQ ID NO: 9); or FGF25 (SEQ ID NO: 35; human FGF1 (SEQ ID NO: 6) with codon usage changes to improve expression in bacteria)).

FIG. 2 shows an exemplary wild-type FGF1 sequence (SEQ ID NO: 14), N-terminal deletions that can be made to FGF1 (SEQ ID NOS: 15-17), point mutations that can be made to FGF1 (SEQ ID NOS: 18-19), and mutations to the heparan binding domain of FGF1 (SEQ ID NOS: 20-21). Also shown are mutations that can be made to FGF2 (SEQ ID NO: 22).

FIGS. 3A-3D show how an exemplary wild-type mature FGF1 sequence (SEQ ID NO: 5) can be mutated to include mutations that increase thermostability of FGF1. FIG. 3A shows an M1 sequence (SEQ ID NO: 36), which can be combined with FGF1 N-terminal deletions and/or point mutations (SEQ ID NOS: 37-41, respectively). FIG. 3B shows an M2 sequence (SEQ ID NO: 42), which can be combined with FGF1 N-terminal deletions and/or point mutations (SEQ ID NOS: 43-52, respectively). FIG. 3C shows an M3 sequence (SEQ ID NO: 54), which can be combined with FGF1 N-terminal deletions and/or point mutations (SEQ ID NOS: 55-61, respectively, and FIG. 3D, SEQ ID NOS: 62-64, respectively). Any of these mutations or FGF1 sequences can be used to make an FGF2/FGF1 chimera.

FIGS. 4A-4B show additional FGF1 mutant sequences that can be generated from an exemplary wild-type mature FGF1 sequence (SEQ ID NO: 14) to include N-terminal deletions and/or point mutations (FIG. 4A, SEQ ID NOS: 66-75, respectively FIG. 4B SEQ ID NOS: 76-80, respectively). Any of these mutations or FGF1 sequences can be used to make an FGF2/FGF1 chimera.

FIGS. 5A-5B show additional FGF1 mutant sequences that can be generated from an exemplary wild-type mature FGF1 sequence (SEQ ID NO: 14) to include N-terminal deletions and/or point mutations (FIG. 5A, SEQ ID NOS: 81-92, respectively, FIG. 5B, SEQ ID NOS: 93-98, respectively). Any of these mutations or FGF1 sequences can be used to make an FGF2/FGF1 chimera.

FIG. 6 shows FGF2/FGF1 chimeras that include FGF2 fragments and FGF1 fragments that include mutations provided herein (SEQ ID NOS: 99-104). The FGF2 portion is in bold. The bracketed sequence highlights sequence differences between human and mouse FGF2. The bracketed (SG) is from the human FGF2 sequence which are replaced by (A) in the murine sequence. FGF24 is a fusion of human FGF2 with human FGF1.

FIG. 7 is a digital image showing the effect of intracellular signaling with M1 thru M5 peptides (SEQ ID NOS: 36, 42, 54, 68, and 19+a C117V mutation respectively). HEK293 cells were serum starved and then treated with the indicated peptides at 10 ng/ml concentration for 15 min. Total cell lysates were subject to western blots with indicated antibodies.

FIG. 8 is a digital image showing peptides NT1 (FGF1^{ΔNT}, SEQ ID NO: 15), NT2 (FGF1^{ΔNT2}, SEQ ID NO: 16), or NT3 (FGF1^{ΔNT3}, SEQ ID NO: 17) KN (SEQ ID NO: 18), and KLE (SEQ ID NO: 19) and their ability to affect intracellular signaling. HEK293 cells were serum starved and then treated with the indicated peptides at 10 ng/ml concentration for 15 min. Total cell lysates were subject to western blots with indicated antibodies.

FIG. 9 is a digital image showing peptides FGF1 (SEQ ID NO: 14) and NT1 (SEQ ID NO: 15) and their ability to affect intracellular signaling. HEK293 cells were serum starved and then treated with the indicated peptides at 10 ng/ml concentration for 15 min. Total cell lysates were subject to western blots with indicated antibodies.

FIGS. 10A and 10B are (A) line and (B) bar graphs showing the glucose lowering effects for M1, M2, and M3 in ob/ob mice. Mice were 5 mo old C57BL/6J ob/ob on normal chow. The peptides were injected SQ (0.5 mg/kg).

FIG. 11 shows in vivo glucose lowering effects correlate with FGFR mediated signaling. Mice were 5 mo old C57BL/6J ob/ob on normal chow. The peptides NT1 (FGF1^{ΔNT}, SEQ ID NO: 15) and NT2 (FGF1^{ΔNT2}, SEQ ID NO: 16), were injected SQ (0.5 mg/kg). The data shows that 9 amino acids can be removed without affecting glucose lowering activity, but removal of an additional 4 amino acids destroys glucose lowering activity.

FIG. 12 is a digital image showing that the in vivo glucose lowering effects correlate with FGFR mediated signaling. Serum starved HEK 293 cells were treated with indicated peptides (10 ng/ml) for 15 min and subject to western blot. FGF1^{ΔNT Prep1} and FGF1^{ΔNT Prep2} are the same sequence (SEQ ID NO: 15), just independent preparations of the protein.

FIG. 13 is a graph showing blood glucose levels 0 to 120 hrs following administration of a single injection of FGF1-KLE (SEQ ID NO: 19) or FGF1-KN (SEQ ID NO: 18). Note that the FGF1-KN mutant retained the ability to lower glucose for 120 hrs while FGF1-KLE fails to lower glucose. Mice were 5 mo old C57BL/6J ob/ob on normal chow. The FGF1-KN and KLE peptides were injected SQ (0.5 mg/kg).

FIG. 14A compares the dose response of downstream FGFR signaling induced by FGF1 and FGF1^{ΔNT} (SEQ ID NO: 15). FIG. 14B is a graph showing food intake in DIO mice after control vehicle (PBS, open bar), rFGF1 (0.5 mg/kg subcutaneous, filled bars), or rFGF1^{ΔNT} (0.5 mg/kg subcutaneous, striped bar) treatment (n=5). FIG. 14C compares the dose response of rFGF1 and NT1 in lowering glucose in ob/ob mice. FIG. 14A. Western blot showing intracellular signaling in serum starved HEK293 cells after a 15 min treatment with the indicated concentrations of PBS (vehicle), rFGF1^{ΔNT}, or rFGF1. FIG. 14B. Food intake in DIO mice during 24 hr period after injection of control vehicle (PBS, open bar), rFGF1 (0.5 mg/kg subcutaneous, filled bars), or rFGF1^{ΔNT} (0.5 mg/kg subcutaneous, striped bar, n=5). FIG. 14C. Dose response of glucose lowering effects of subcutaneously delivered rFGF1^{ΔNT} (striped bars) in comparison to rFGF1 (filled bars) in 12 week old ob/ob mice (n=6-12). ***P<0.005.

FIG. 15 is a bar graph showing blood glucose levels 0 hr, 16 hrs, or 24 hrs following administration of PBS, NT1 (SEQ ID NO: 15), NT2 (SEQ ID NO: 16), or NT3 (SEQ ID NO: 17). Note that if the N-terminus is truncated at 14 amino acids, glucose lowering ability is decreased (NT2). Mice were 5 mo old C57BL/6J ob/ob on normal chow. The peptides were injected SQ (0.5 mg/kg).

FIGS. 16A and 16B are bar graphs showing that NT1 (SEQ ID NO: 15) fails to lower blood glucose levels in HFD-fed aP2-Cre; FGFR1 f/f mice (mutant FGFR1 KO mice). Blood glucose levels in 8 month old HFD-fed wildtype FGFR1 f/f (control open bars) or adipose-specific FGFR1 knockout (mutant, R1 KO, aP2-Cre; FGFR1 f/f, filled bars) mice after NT1 treatment (murine rFGF1^{ΔNT}, 0.5 mg/kg subcutaneous injection, n=5 per group). Values are means±SEM. FIG. 16A shows the raw blood glucose levels, FIG. 16B shows the data normalized to initial blood glucose as 100%.

FIGS. 17A and 17B are bar graphs showing that recombinant mouse rFGF1 (amino acids 15-155 of SEQ ID NO: 8; murine FGF1 is ˜96% homologous to the human sequence, amino acids 15-155 of SEQ ID NO: 6) fails to lower blood glucose levels in HFD-fed aP2-Cre; FGFR1 f/f mice (FGFR1 KO, filled bars). Blood glucose levels in 8 month old HFD-fed wild type (FGFR1 f/f, black bars) or adipose-specific FGFR1 knockout (R1 KO, aP2-Cre; FGFR1f/f, dotted bars) mice after rFGF1 treatment (murine rFGF1, 0.5 mg/kg subcutaneous injection, n-=5 per group). Values are means±SEM. FIG. 17A shows the raw blood glucose levels, FIG. 17B shows the data normalized to initial blood glucose as 100%.

FIG. 18 is a bar graph showing that FGF1 incorporating the mutations K118E (SEQ ID NO: 20) and K118N (SEQ ID NO: 21) fail to lower blood glucose levels in DIO mice. Blood glucose levels in 7 months HFD-fed C57BL/6J mice after PBS (open bar), K118E (filled bar), and K118N (hatched bar) treatment (0.5 mg/kg subcutaneous injection, n=4-8 per group). Values are means±SEM.

FIGS. 19A-19F show exemplary arrangements of FGF2/FGF1/β-Klotho-binding chimeras, FGF2/FGF1/FGFR1c-binding chimeras, and FGF2/FGF1/β-Klotho-binding/FGFR1c-binding chimeras, with the FGF2/FGF1 portion at the N-terminus, such as (A) FGF2/FGF1/β-Klotho-binding chimera, (B) FGF2/FGF1/β-Klotho-binding/β-Klotho-binding chimera, (C) FGF2/FGF1/FGFR1c-binding chimera, (D) FGF2/FGF1/FGFR1c-binding/FGFR1c-binding chimera, (E) FGF2/FGF1/β-Klotho-binding/FGFR1c-binding chimera and (F) FGF2/FGF1/FGFR1c-binding/β-Klotho-binding chimera. Although monomers and dimers of FGFR1c- or β-Klotho-binding proteins are shown, in some examples greater multimers are used, such as trimers, etc.

FIGS. 20A-20F show exemplary arrangements of FGF2/FGF1/β-Klotho-binding chimeras, FGF2/FGF1/FGFR1c-binding chimeras, and FGF2/FGF1/β-Klotho-binding/FGFR1c-binding chimeras, with the FGF2/FGF1 portion at the C-terminus, such as (A) β-Klotho-binding/FGF2/FGF1 chimera, (B) β-Klotho-binding/β-Klotho-binding/FGF2/FGF1/chimera, (C) FGFR1c-binding/FGF2/FGF1 chimera, (D) FGFR1c-binding/FGFR1c-binding/FGF2/FGF1 chimera, (E) FGFR1c-binding/β-Klotho-binding/FGF2/FGF1 chimera and (F) β-Klotho-binding/FGFR1c-binding/FGF2/FGF1 chimera. Although monomers and dimers of FGFR1c- or β-Klotho-binding proteins are shown, in some examples greater multimers are used, such as trimers, etc.

FIGS. 21A-21F show exemplary arrangements of FGF2/FGF1/β-Klotho-binding chimeras (with C2240 as the β-Klotho-binding sequence), FGF2/FGF1/FGFR1c-binding chimeras (with C2987 as the FGFR1c-binding sequence), and FGF2/FGF1/β-Klotho-binding/FGFR1c-binding chimeras, with the FGF2/FGF1 portion at the N-terminus, such as (A) FGF2/FGF1/β-Klotho-binding chimera, (B) FGF2/FGF1/β-Klotho-binding/β-Klotho-binding chimera, (C) FGF2/FGF1/FGFR1c-binding chimera, (D) FGF2/FGF1/FGFR1c-binding/FGFR1c-binding chimera, (E) FGF2/FGF1/β-Klotho-binding/FGFR1c-binding chimera and (F) FGF2/FGF1/FGFR1c-binding/β-Klotho-binding chimera. Exemplary sequences are shown in SEQ ID NOS: 156-161. Although SEQ ID NO: 102 is used for the FGF2/FGF1 portion, other FGF2/FGF1 chimeras provided herein can be used. Although monomers and dimers of FGFR1c- or β-Klotho-binding proteins are shown, in some examples greater multimers are used, such as trimers, etc.

FIGS. 22A-22F show exemplary arrangements of FGF2/FGF1/β-Klotho-binding chimeras (with C2240 as the β-Klotho-binding sequence), FGF2/FGF1/FGFR1c-binding chimeras (with C2987 as the FGFR1c-binding sequence), and FGF2/FGF1/β-Klotho-binding/FGFR1c-binding chimeras, with the FGF2/FGF1 portion at the C-terminus, such as (A) β-Klotho-binding/FGF2/FGF1 chimera, (B) β-Klotho-binding/β-Klotho-binding/FGF2/FGF1/chimera, (C) FGFR1c-binding/FGF2/FGF1 chimera, (D) FGFR1c-binding/FGFR1c-binding/FGF2/FGF1 chimera, (E) FGFR1c-binding/β-Klotho-binding/FGF2/FGF1 chimera and (F) β-Klotho-binding/FGFR1c-binding/FGF2/FGF1 chimera. Exemplary sequences are shown in SEQ ID NOS: 162-167. Although SEQ ID NO: 102 is used for the FGF2/FGF1 portion, other FGF2/FGF1 chimeras provided herein can be used. Although monomers and dimers of FGFR1c- or β-Klotho-binding proteins are shown, in some examples greater multimers are used, such as trimers, etc.

FIG. 23 shows a native FGF1 sequence (SEQ ID NO: 14) and eight heparan binding mutant FGF1 KKK analogs (SEQ ID NOS: 168, 169, 170, 171, 172, 173, 174, and 175). The FGF2/FGF1 chimeras provided herein can include the FGF1 mutations shown in these sequences.

FIGS. 24 and 25 show that FGF1 heparan binding mutant KKK lowers glucose.

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NOS: 1 and 2 provide exemplary human FGF2 nucleic acid and protein sequences, respectively. Source: GenBank® Accession Nos: AB451450 and BAG70264, respectively. Heparan binding residues are amino acids 128, 129, 134, 138, and 143-145 of SEQ ID NO: 2.

SEQ ID NOS: 3 and 4 provide an exemplary mouse FGF2 nucleic acid and protein sequences, respectively. Source: GenBank® Accession Nos: NM_008006 and NP_032032.

SEQ ID NOS: 5 and 6 provide an exemplary human FGF1 nucleic acid and protein sequences, respectively. Source: GenBank® Accession Nos: BC032697.1 and AAH32697.1. Heparan binding residues are amino acids 127-129 and 133-134 of SEQ ID NO: 6.

SEQ ID NOS: 7 and 8 provide an exemplary mouse FGF1 nucleic acid and protein sequences, respectively. Source: GenBank® Accession Nos: BC037601.1 and AAH37601.1.

SEQ ID NO: 9 provides an exemplary FGF2/FGF1 chimeric protein sequence (FGF24). The FGF2 portion is amino acids 1-21 and the FGF1 portion is amino acids 22-158.

SEQ ID NO: 10 provides an exemplary FGF2/FGF1 chimeric protein sequence (FGF24.1). The FGF2 portion is amino acids 1-21 and the FGF1 portion is amino acids 22-158.

SEQ ID NO: 11 provides an exemplary FGF2/FGF1 chimeric protein sequence (FGF24.2). The FGF2 portion is amino acids 1-21 and the FGF1 portion is amino acids 22-158.

SEQ ID NO: 12 provides an exemplary FGF2/FGF1 chimeric protein sequence (FGF24 without the MAAGSITTL signal sequence). The FGF2 portion is amino acids 1-12 and the FGF1 portion is amino acids 13-149.

SEQ ID NO: 13 provides an exemplary FGF2/FGF1 chimeric protein sequence (FGF24 with an N-terminal M instead of the MAAGSITTL signal sequence). The FGF2 portion is amino acids 1-13 and the FGF1 portion is amino acids 14-150.

SEQ ID NO: 14 provides an exemplary mature form of FGF1 (140 aa, sometimes referred to in the art as FGF1 15-154)

SEQ ID NO: 15 provides an exemplary mature form of FGF1 with an N-terminal deletion.

SEQ ID NO: 16 provides an exemplary mature form of FGF1 with an N-terminal deletion.

SEQ ID NO: 17 provides an exemplary mature form of FGF1 with an N-terminal deletion.

SEQ ID NO: 18 provides an exemplary mature form of FGF1 with point mutations (K12V, N95V, wherein numbering refers to SEQ ID NO: 14) to reduce mitogenic activity.

SEQ ID NO: 19 provides an exemplary mature form of FGF1 with point mutations (K12V, L46V, E87V, N95V, P134V, wherein numbering refers to SEQ ID NO: 14) to reduce mitogenic activity.

SEQ ID NOS: 20 and 21 provide exemplary mature forms of FGF1 with mutations in the heparan binding domain (K118N or K118E, respectively, wherein numbering refers to SEQ ID NO: 14). In some examples these sequences further include MFNLPPG at their N-terminus. Such proteins have reduced mitogenicity as compared to wild-type FGF1.

SEQ ID NO: 22 provides an exemplary FGF2 with point mutations (G19F, H25N, F26Y, numbering refers to SEQ ID NO: 2). These mutations allow FG2 to bind to all FGF receptors (e.g., see Beenken et al., J. Biol. Chem. 287(5):3067-78, 2012).

SEQ ID NOS: 23-26 provide exemplary mutated FGF1 nuclear export sequences.

SEQ ID NO: 27 provides an exemplary FGF1 sequence that is non-mitogenic.

SEQ ID NO: 28 provides an exemplary mouse FGF2 with an N-terminal truncation, and with a methionine added to the N-terminus.

SEQ ID NO: 29 provides an exemplary mouse FGF2 protein sequence with three point mutations (at amino acids 9, 10 and 11; changed to the equivalent residues in FGF1 to allow the chimeric protein to bind to all FGF receptors).

SEQ ID NO: 30 provides an exemplary human FGF2 protein sequence with three point mutations (G19F, H25N, F26Y, wherein numbering refers to SEQ ID NO: 2, changed to the equivalent residues in FGF1 to allow the chimeric protein to bind to all FGF receptors).

SEQ ID NO: 31 provides a coding sequence for FGF24 (SEQ ID NO: 9).

SEQ ID NO: 32 provides a coding sequence for FGF24.1 (SEQ ID NO: 10).

SEQ ID NO: 33 provides a coding sequence for FGF24.2 (SEQ ID NO: 11).

SEQ ID NOS: 34 and 35 provide a coding sequence and protein sequence for FGF25.

SEQ ID NO: 36 provides an exemplary mature form of FGF1 with point mutations (K12V, C117V and P134V wherein numbering refers to SEQ ID NO: 14) to reduce mitogenic activity and increase thermostability. From Xia et al., PLoS One. 7 (11):e48210, 2012.

SEQ ID NO: 37 (FGF1(1-140αα)M1a) provides an exemplary mature form of FGF1 with point mutations (K12V, N95V, C117V, and P134V wherein numbering refers to SEQ ID NO: 14) to reduce mitogenic activity and increase thermostability.

SEQ ID NO: 38 (FGF1^{ΔNT1} (1-140αα)M1) provides an exemplary N-terminally truncated form of FGF1 with point mutations (K12V, C117V, and P134V wherein numbering refers to SEQ ID NO: 14) to reduce mitogenic activity and increase thermostability.

SEQ ID NO: 39 (FGF1^{ΔNT3} (1-140αα)M1a) provides an exemplary N-terminally truncated form of FGF1 with point mutations (K12V, C117V, and P134V wherein numbering refers to SEQ ID NO: 14) to reduce mitogenic activity and increase thermostability.

SEQ ID NO: 40 (FGF1^{ΔNT1} (1-140αα)M1a) provides an exemplary N-terminally truncated form of FGF1 with point mutations (K12V, N95V, C117V, and P134V wherein numbering refers to SEQ ID NO: 14) to reduce mitogenic activity, and increase thermostability

SEQ ID NO: 41 (FGF1^{ΔNT3} (1-140αα)M1a) provides an exemplary N-terminally truncated form of FGF1 with point mutations (K12V, N95V, C117V, and P134V wherein numbering refers to SEQ ID NO: 14) to reduce mitogenic activity, and increase thermostability

SEQ ID NO: 42 (FGF1(1-140αα)M2) provides an exemplary mature form of FGF1 with point mutations (L44F, C83T, C117V, and F132W wherein numbering refers to SEQ ID NO: 14) to reduce mitogenic activity and increase thermostability. From Xia et al., PLoS One. 7 (11):e48210, 2012.

SEQ ID NO: 43 (FGF1(1-140αα)M2a) provides an exemplary mature form of FGF1 with point mutations (L44F, C83T, N95V, C117V, and F132W wherein numbering refers to SEQ ID NO: 14) to reduce mitogenic activity and increase thermostability.

SEQ ID NO: 44 (FGF1(1-140αα)M2b) provides an exemplary mature form of FGF1 with point mutations (K12V, L44F, C83T, C117V, and F132W wherein numbering refers to SEQ ID NO: 14) to reduce mitogenic activity and increase thermostability.

SEQ ID NO: 45 (FGF1(1-140αα)M2c) provides an exemplary mature form of FGF1 with point mutations (K12V, L44F, C83T, N95V, C117V, and F132W wherein numbering refers to SEQ ID NO: 14) to reduce mitogenic activity and increase thermostability.

SEQ ID NO: 46 (FGF1^{ΔNT1}(10-140αα)M2) provides an exemplary N-terminally truncated form of FGF1 with point mutations (L44F, C83T, C117V, and F132W wherein numbering refers to SEQ ID NO: 14) to reduce mitogenic activity and increase thermostability.

SEQ ID NO: 47 (FGF1^{ΔNT3}(12-140αα)M2) provides an exemplary N-terminally truncated form of FGF1 with point mutations (L44F, C83T, C117V, and F132W wherein numbering refers to SEQ ID NO: 14) to reduce mitogenic activity and increase thermostability.

SEQ ID NO: 48 (FGF1^{ΔNT1}(10-140αα)M2a) provides an exemplary N-terminally truncated form of FGF1 with point mutations (L44F, C83T, N95V, C117V, and F132W wherein numbering refers to SEQ ID NO: 14) to reduce mitogenic activity and increase thermostability.

SEQ ID NO: 49 (FGF1^{ΔNT3}(12-140αα)M2a) provides an exemplary N-terminally truncated form of FGF1 with point mutations (L44F, C83T, N95V, C117V, and F132W wherein numbering refers to SEQ ID NO: 14) to reduce mitogenic activity and increase thermostability.

SEQ ID NO: 50 (FGF1^{ΔNT1}(10-140αα)M2b) provides an exemplary N-terminally truncated form of FGF1 with point mutations (K12V, L44F, C83T, C117V, and F132W wherein numbering refers to SEQ ID NO: 14) to reduce mitogenic activity and increase thermostability.

SEQ ID NO: 51 (FGF1^{ΔNT3}(12-140αα)M2b) provides an exemplary N-terminally truncated form of FGF1 with point mutations (K12V, L44F, C83T, C117V, and F132W wherein numbering refers to SEQ ID NO: 14) to reduce mitogenic activity and increase thermostability.

SEQ ID NO: 52 (FGF1^{ΔNT1}(10-140αα)M2c) provides an exemplary N-terminally truncated form of FGF1 with point mutations (K12V, L44F, C83T, N95V, and C117V, F132W wherein numbering refers to SEQ ID NO: 14) to reduce mitogenic activity and increase thermostability.

SEQ ID NO: 53 (FGF1^{ΔNT3}(12-140αα)M2c) provides an exemplary N-terminally truncated form of FGF1 with point mutations (K12V, L44F, C83T, N95V, and C117V, F132W wherein numbering refers to SEQ ID NO: 14) to reduce mitogenic activity and increase thermostability.

SEQ ID NO: 54 (FGF1(1-140αα)M3) provides an exemplary mature form of FGF1 with mutations (L44F, M67I, L73V, V109L, L111I, C117V, A103G, R119G Δ104-106, and Δ120-122, wherein numbering refers to SEQ ID NO: 14) to reduce mitogenic activity and increase thermostability. From Xia et al., PLoS One. 7(11):e48210, 2012.

SEQ ID NO: 55 (FGF1(1-140αα)M3a) provides an exemplary mature form of FGF1 with mutations (K12V, L44F, M67I, L73V, V109L, L111I, C117V, A103G, R119G, Δ104-106, and Δ120-122 wherein numbering refers to SEQ ID NO: 14) to reduce mitogenic activity and increase thermostability.

SEQ ID NO: 56 (FGF1(1-140αα)M3b) provides an exemplary mature form of FGF1 with mutations (K12V, L44F, M67I, L73V, N95V, V109L, L111I, C117V, A103G, R119G, Δ104-106, and Δ120-122 wherein numbering refers to SEQ ID NO: 14) to reduce mitogenic activity and increase thermostability.

SEQ ID NO: 57 (FGF1(1-140αα)M3c) provides an exemplary mature form of FGF1 with mutations (K12V, L44F, M67I, L73V, N95V, V109L, L111I, C117V, A103G, R119G, Δ104-106, and Δ120-122 wherein numbering refers to SEQ ID NO: 14) to reduce mitogenic activity and increase thermostability.

SEQ ID NO: 58 (FGF1^{ΔNT1} (1-140αα)M3) provides an exemplary N-terminally truncated form of FGF1 with mutations (L44F, M67I, L73V, V109L, L111I, C117V, A103G, R119G, Δ104-106, and Δ120-122 wherein numbering refers to SEQ ID NO: 14) to reduce mitogenic activity and increase thermostability.

SEQ ID NO: 59 (FGF1^{ΔNT3} (1-140αα)M3) provides an exemplary N-terminally truncated form of FGF1 with mutations (L44F, M67I, L73V, V109L, L111I, C117V, A103G, R119G, Δ104-106, and Δ120-122 wherein numbering refers to SEQ ID NO: 14) to reduce mitogenic activity and increase thermostability.

SEQ ID NO: 60 (FGF1^{ΔNT1} (1-140αα)M3a) provides an exemplary N-terminally truncated form of FGF1 with mutations (K12V, L44F, M67I, L73V, V109L, L111I, C117V, A103G, R119G, Δ104-106, and Δ120-122 wherein numbering refers to SEQ ID NO: 14) to reduce mitogenic activity and increase thermostability.

SEQ ID NO: 61 (FGF1^{ΔNT3} (1-140αα)M3a) provides an exemplary N-terminally truncated form of FGF1 with mutations (K12V, L44F, M67I, L73V, V109L, L111I, C117V, A103G, R119G, Δ104-106, and Δ120-122 wherein numbering refers to SEQ ID NO: 14) to reduce mitogenic activity and increase thermostability.

SEQ ID NO: 62 (FGF1^{ΔNT1} (1-140αα)M3b) provides an exemplary N-terminally truncated form of FGF1 with mutations (L44F, M67I, L73V, N95V, V109L, L111I, C117V, A103G, R119G, Δ104-106, and Δ120-122 wherein numbering refers to SEQ ID NO: 14) to reduce mitogenic activity and increase thermostability.

SEQ ID NO: 63 (FGF1^{ΔNT3} (1-140αα)M3b) provides an exemplary N-terminally truncated form of FGF1 with mutations (L44F, M67I, L73V, N95V, V109L, L111I, C117V, A103G, R119G, Δ104-106, and Δ120-122 wherein numbering refers to SEQ ID NO: 14) to reduce mitogenic activity and increase thermostability.

SEQ ID NO: 64 (FGF1^{ΔNT1} (1-140αα)M3c) provides an exemplary N-terminally truncated form of FGF1 with mutations (K12V, L44F, M67I, L73V, N95V, V109L, L111I, C117V, A103G, R119G, Δ104-106, and Δ120-122 wherein numbering refers to SEQ ID NO: 14) to reduce mitogenic activity and increase thermostability.

SEQ ID NO: 65 (FGF1^{ΔNT3} (1-140αα)M3c) provides an exemplary N-terminally truncated form of FGF1 with point mutations (K12V, L44F, M67I, L73V, N95V, V109L, L111I, C117V, A103G, R119G, Δ104-106, and Δ120-122 wherein numbering refers to SEQ ID NO: 14) to reduce mitogenic activity and increase thermostability.

SEQ ID NO: 66 (FGF1 (1-140αα) provides an exemplary mature form of FGF1 with point mutations (K12V, N95V, and K118N wherein numbering refers to SEQ ID NO: 14).

SEQ ID NO: 67 (FGF1 (1-140αα) provides an exemplary mature form of FGF1 with point mutations (K12V, N95, and K118E wherein numbering refers to SEQ ID NO: 14).

SEQ ID NO: 68 (FGF1 (1-140αα) provides an exemplary mature form of FGF1 with point mutations (K12V, N95V, and C117V wherein numbering refers to SEQ ID NO: 14).

SEQ ID NO: 69 (FGF1 (1-140αα) provides an exemplary mature form of FGF1 with point mutations (K12V, N95V, C117V, and K118N wherein numbering refers to SEQ ID NO: 14).

SEQ ID NO: 70 (FGF1 (1-140αα) provides an exemplary mature form of FGF1 with point mutations (K12V, N95V, C117V, and K118E wherein numbering refers to SEQ ID NO: 14).

SEQ ID NO: 71 (FGF1^{ΔNT} (10-140αα) provides an exemplary N-terminally truncated FGF1 with point mutations (K12V and N95V, wherein numbering refers to SEQ ID NO: 14).

SEQ ID NO: 72 (FGF1^{ΔNT2} (12-140αα) provides an exemplary N-terminally truncated FGF1 with point mutations (K12V, and N95V, wherein numbering refers to SEQ ID NO: 14).

SEQ ID NO: 73 (FGF1^{ΔNT} (10-140αα) provides an exemplary N-terminally truncated FGF1 with a point mutation (K12V, wherein numbering refers to SEQ ID NO: 14).

SEQ ID NO: 74 (FGF1^{ΔNT2} (12-140αα) provides an exemplary N-terminally truncated FGF1 with a point mutation (K12V, wherein numbering refers to SEQ ID NO: 14).

SEQ ID NO: 75 (FGF1^{ΔNT} (10-140αα) provides an exemplary N-terminally truncated FGF1 with a point mutation (N95V, wherein numbering refers to SEQ ID NO: 14).

SEQ ID NO: 76 (FGF1^{ΔNT2} (12-140αα) provides an exemplary N-terminally truncated FGF1 with a point mutation (N95V, wherein numbering refers to SEQ ID NO: 14).

SEQ ID NO: 77 (FGF1^{ΔNT} (10-140αα) provides an exemplary N-terminally truncated FGF1 with point mutations (K12V, N95V, and K118N, wherein numbering refers to SEQ ID NO: 14).

SEQ ID NO: 78 (FGF1^{ΔNT2} (12-140αα) provides an exemplary N-terminally truncated FGF1 with point mutations (K12V, N95V, and K118E, wherein numbering refers to SEQ ID NO: 14).

SEQ ID NO: 79 (FGF1^{ΔNT} (10-140αα) provides an exemplary N-terminally truncated FGF1 with a point mutation (K118N, wherein numbering refers to SEQ ID NO: 14).

SEQ ID NO: 80 (FGF1^{ΔNT2} (12-140αα) provides an exemplary N-terminally truncated FGF1 with a point mutation (K118E, wherein numbering refers to SEQ ID NO: 14).

SEQ ID NO: 81 (FGF1 (1-140αα) provides an exemplary mature form of FGF1 with point mutations (K9T and N10T wherein numbering refers to SEQ ID NO: 14).

SEQ ID NO: 82 (FGF1 (1-140αα) provides an exemplary mature form of FGF1 with point mutations (K9T, N10T, and N95V, wherein numbering refers to SEQ ID NO: 14).

SEQ ID NO: 83 (FGF1 (1-140αα) provides an exemplary mature form of FGF1 with point mutations (K9T, N10T, and K118N, wherein numbering refers to SEQ ID NO: 14).

SEQ ID NO: 84 (FGF1 (1-140αα) provides an exemplary mature form of FGF1 with a mutant NLS sequence.

SEQ ID NO: 85 (FGF1^{ΔNT} (1-140αα) provides an exemplary N-terminally truncated form of FGF1 with point mutations (Q40P and S47I, wherein numbering refers to SEQ ID NO: 14).

SEQ ID NO: 86 (FGF1^{ΔNT3} (1-140αα) provides an exemplary N-terminally truncated form of FGF1 with point mutations (Q40P and S47I, wherein numbering refers to SEQ ID NO: 14).

SEQ ID NO: 87 (FGF1 (1-140αα) provides an exemplary mature form of FGF1 with point mutations (K12V, Q40P, S47I, and N95V wherein numbering refers to SEQ ID NO: 14).

SEQ ID NO: 88 FGF1^{ΔNT} (1-140αα) provides an exemplary N-terminally truncated form of FGF1 with point mutations (K12V, Q40P, S47I, and N95V, wherein numbering refers to SEQ ID NO: 14).

SEQ ID NO: 89 (FGF1^{ΔNT3} (1-140αα) provides an exemplary N-terminally truncated form of FGF1 with point mutations (K12V, Q40P, S47I, and N95V, wherein numbering refers to SEQ ID NO: 14).

SEQ ID NO: 90 (FGF1^{ΔNT} (1-140αα) provides an exemplary N-terminally truncated form of FGF1 with point mutations (Q40P, S47I, and H93G, wherein numbering refers to SEQ ID NO: 14).

SEQ ID NO: 91 (FGF1^{ΔNT3} (1-140αα) provides an exemplary N-terminally truncated form of FGF1 with point mutations (Q40P, S47I, and H93G, wherein numbering refers to SEQ ID NO: 14).

SEQ ID NO: 92 (FGF1 (1-140αα) provides an exemplary mature form of FGF1 with point mutations (K12V, Q40P, S47I, H93G, and N95V, wherein numbering refers to SEQ ID NO:14).

SEQ ID NO: 93 (FGF1^{ΔNT} (1-140αα) provides an exemplary N-terminally truncated form of FGF1 with point mutations (K12V, Q40P, S47I, H93G, and N95V, wherein numbering refers to SEQ ID NO: 14).

SEQ ID NO: 94 (FGF1^{ΔNT3} (1-140αα) provides an exemplary N-terminally truncated form of FGF1 with point mutations (K12V, Q40P, S47I, H93G, and N95V, wherein numbering refers to SEQ ID NO: 14).

SEQ ID NO: 95 (FGF1^{ΔNT} (1-140αα) provides an exemplary N-terminally truncated form of FGF1 with point mutations (C117P and K118V, wherein numbering refers to SEQ ID NO: 14).

SEQ ID NO: 96 (FGF1^{ΔNT3} (1-140αα) provides an exemplary N-terminally truncated form of FGF1 with point mutations (C117P and K118V, wherein numbering refers to SEQ ID NO: 14).

SEQ ID NO: 97 (FGF1 (1-140αα) provides an exemplary mature form of FGF1 with point mutations (K12V, N95V, C117P, and K118V, wherein numbering refers to SEQ ID NO: 14).

SEQ ID NO: 98 (FGF1 (1-140αα) provides an exemplary mature form of FGF1 with a point mutation (R35E, wherein numbering refers to SEQ ID NO: 14).

SEQ ID NOS: 99-104 provide exemplary FGF2/FGF1 chimeric protein sequences with reduced or no mitogenic activity.

SEQ ID NO: 105 provides an exemplary β-Klotho binding protein dimer sequence (C2240) that can be attached at its N- or C-terminus directly or indirectly to any of the FGF2/FGF1 chimeras provided herein.

SEQ ID NO: 106 provides an exemplary β-Klotho binding protein sequence that can be attached at its N- or C-terminus directly or indirectly to any of the FGF2/FGF1 chimeras provided herein.

SEQ ID NO: 107 provides an exemplary β-Klotho binding protein sequence that can be attached at its N- or C-terminus directly or indirectly to any of the FGF2/FGF1 chimeras provided herein. In addition, it can be linked to SEQ ID NO: 106 via a linker and then the resulting chimera attached at its N- or C-terminus directly or indirectly to any of the FGF1 mutants provided herein to generate a chimeric protein.

SEQ ID NO: 108 provides an exemplary β-Klotho binding protein sequence that can be attached at its N- or C-terminus directly or indirectly to any of the FGF2/FGF1 chimeras provided herein. In addition, it can be linked to SEQ ID NO: 106 via a linker and then the resulting chimera attached at its N- or C-terminus directly or indirectly to any of the FGF2/FGF1 chimeras provided herein.

SEQ ID NO: 109 provides an exemplary β-Klotho binding protein sequence that can be attached at its N- or C-terminus directly or indirectly to any of the FGF2/FGF1 chimeras provided herein. In addition, it can be linked to SEQ ID NO: 106 via a linker and then the resulting chimera attached at its N- or C-terminus directly or indirectly to any of the FGF2/FGF1 chimeras provided herein.

SEQ ID NO: 110 provides an exemplary β-Klotho binding protein sequence that can be attached at its N- or C-terminus directly or indirectly to any of the FGF2/FGF1 chimeras provided herein. In addition, it can be linked to SEQ ID NO: 106 via a linker and then the resulting chimera attached at its N- or C-terminus directly or indirectly to any of the FGF2/FGF1 chimeras provided herein.

SEQ ID NO: 111 provides an exemplary β-Klotho binding protein sequence that can be attached at its N- or C-terminus directly or indirectly to any of the FGF2/FGF1 chimeras provided herein. In addition, it can be linked to SEQ ID NO: 106 via a linker and then the resulting chimera attached at its N- or C-terminus directly or indirectly to any of the FGF2/FGF1 chimeras provided herein.

SEQ ID NO: 112 provides an exemplary β-Klotho binding protein sequence can be attached at its N- or C-terminus directly or indirectly to any of the FGF2/FGF1 chimeras provided herein. In addition, it can be linked to SEQ ID NO: 106 via a linker and then the resulting chimera attached at its N- or C-terminus directly or indirectly to any of the FGF2/FGF1 chimeras provided herein.

SEQ ID NO: 113 provides an exemplary β-Klotho binding protein sequence can be attached at its N- or C-terminus directly or indirectly to any of the FGF2/FGF1 chimeras provided herein. In addition, it can be linked to SEQ ID NO: 106 via a linker and then the resulting chimera attached at its N- or C-terminus directly or indirectly to any of the FGF2/FGF1 chimeras provided herein.

SEQ ID NO: 114 provides an exemplary β-Klotho binding protein sequence can be attached at its N- or C-terminus directly or indirectly to any of the FGF2/FGF1 chimeras provided herein. In addition, it can be linked to SEQ ID NO: 106 via a linker and then the resulting chimera attached at its N- or C-terminus directly or indirectly to any of the FGF2/FGF1 chimeras provided herein.

SEQ ID NO: 115 provides an exemplary β-Klotho binding protein sequence that can be attached at its N- or C-terminus directly or indirectly to any of the FGF2/FGF1 chimeras provided herein.

SEQ ID NO: 116 provides an exemplary β-Klotho binding protein sequence that can be attached at its N- or C-terminus directly or indirectly to any of the FGF2/FGF1 chimeras provided herein.

SEQ ID NO: 117 provides an exemplary β-Klotho binding protein sequence that can be attached at its N- or C-terminus directly or indirectly to any of the FGF2/FGF1 chimeras provided herein.

SEQ ID NO: 118 provides an exemplary β-Klotho binding protein sequence that can be attached at its N- or C-terminus directly or indirectly to any of the FGF2/FGF1 chimeras provided herein.

SEQ ID NO: 119 provides an exemplary β-Klotho binding protein sequence that can be attached at its N- or C-terminus directly or indirectly to any of the FGF2/FGF1 chimeras provided herein.

SEQ ID NO: 120 provides an exemplary β-Klotho binding protein sequence that can be attached at its N- or C-terminus directly or indirectly to any of the FGF2/FGF1 chimeras provided herein.

SEQ ID NO: 121 provides an exemplary β-Klotho binding protein sequence that can be attached at its N- or C-terminus directly or indirectly to any of the FGF2/FGF1 chimeras provided herein.

SEQ ID NO: 122 provides an exemplary β-Klotho binding protein sequence that can be attached at its N- or C-terminus directly or indirectly to any of the FGF2/FGF1 chimeras provided herein.

SEQ ID NO: 123 provides an exemplary β-Klotho binding protein sequence that can be attached at its N- or C-terminus directly or indirectly to any of the FGF2/FGF1 chimeras provided herein.

SEQ ID NO: 124 provides an exemplary β-Klotho binding protein sequence that can be attached at its N- or C-terminus directly or indirectly to any of the FGF2/FGF1 chimeras provided herein.

SEQ ID NO: 125 provides an exemplary β-Klotho binding protein sequence that can be attached at its N- or C-terminus directly or indirectly to any of the FGF2/FGF1 chimeras provided herein. In addition, it can be linked to any of SEQ ID NOS: 126-127 via a linker and then the resulting chimera attached at its N- or C-terminus directly or indirectly to any of the FGF2/FGF1 chimeras provided herein.

SEQ ID NO: 126 provides an exemplary β-Klotho binding protein sequence that can be attached at its N- or C-terminus directly or indirectly to any of the FGF2/FGF1 chimeras provided herein. In addition, it can be linked to SEQ ID NO: 125 via a linker and then the resulting chimera attached at its N- or C-terminus directly or indirectly to any of the FGF2/FGF1 chimeras provided herein.

SEQ ID NO: 127 provides an exemplary β-Klotho binding protein sequence that can be attached at its N- or C-terminus directly or indirectly to any of the FGF2/FGF1 chimeras provided herein. In addition, it can be linked to SEQ ID NO: 125 via a linker and then the resulting chimera attached at its N- or C-terminus directly or indirectly to any of the FGF2/FGF1 chimeras provided herein.

SEQ ID NO: 128 provides an exemplary β-Klotho binding protein sequence that can be attached at its N- or C-terminus directly or indirectly to any of the FGF2/FGF1 chimeras provided herein.

SEQ ID NO: 129 provides an exemplary β-Klotho binding protein sequence that can be attached at its N- or C-terminus directly or indirectly to any of the FGF2/FGF1 chimeras provided herein.

SEQ ID NO: 130 provides an exemplary β-Klotho binding protein sequence that can be attached at its N- or C-terminus directly or indirectly to any of the FGF2/FGF1 chimeras provided herein.

SEQ ID NO: 131 provides an exemplary FGFR1c binding protein sequence that can be attached at its N- or C-terminus directly or indirectly to any of the FGF1 mutants provided herein to generate a chimeric protein. In addition, it can be linked to itself one or more times to generate an FGFR1c multimer, such as a dimer or a trimer.

SEQ ID NO: 132 (C2987) provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 133 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 134 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 135 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 136 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 137 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 138 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 139 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 140 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 141 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 142 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 143 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 144 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 145 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 146 provides an exemplary FGFR1c binding protein sequence that can be attached at its N- or C-terminus directly or indirectly to any of the FGF2/FGF1 chimeras provided herein. In addition, it can be linked to itself one or more times to generate an FGFr1c multimer, such as a dimer or a trimer.

SEQ ID NO: 147 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 148 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 149 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 150 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 151 provides an exemplary FGFR1c binding protein sequence that can be attached at its N- or C-terminus directly or indirectly to any of the FGF2/FGF1 chimeras provided herein. In addition, it can be linked to itself one or more times to generate an FGFR1c multimer, such as a dimer or a trimer.

SEQ ID NO: 152 provides an exemplary β-Klotho-FGFR1c binding protein sequence that can be attached at its N- or C-terminus directly or indirectly to any of the FGF2/FGF1 chimeras provided herein.

SEQ ID NO: 153 provides an exemplary β-Klotho-FGFR1c binding protein sequence that can be attached at its N- or C-terminus directly or indirectly to any of the FGF2/FGF1 chimeras provided herein.

SEQ ID NO: 154 provides an exemplary β-Klotho-FGFR1c binding protein sequence that can be attached at its N- or C-terminus directly or indirectly to any of the FGF2/FGF1 chimeras provided herein.

SEQ ID NO: 155 provides an exemplary β-Klotho-FGFR1c binding protein sequence that can be attached at its N- or C-terminus directly or indirectly to any of the FGF2/FGF1 chimeras provided herein.

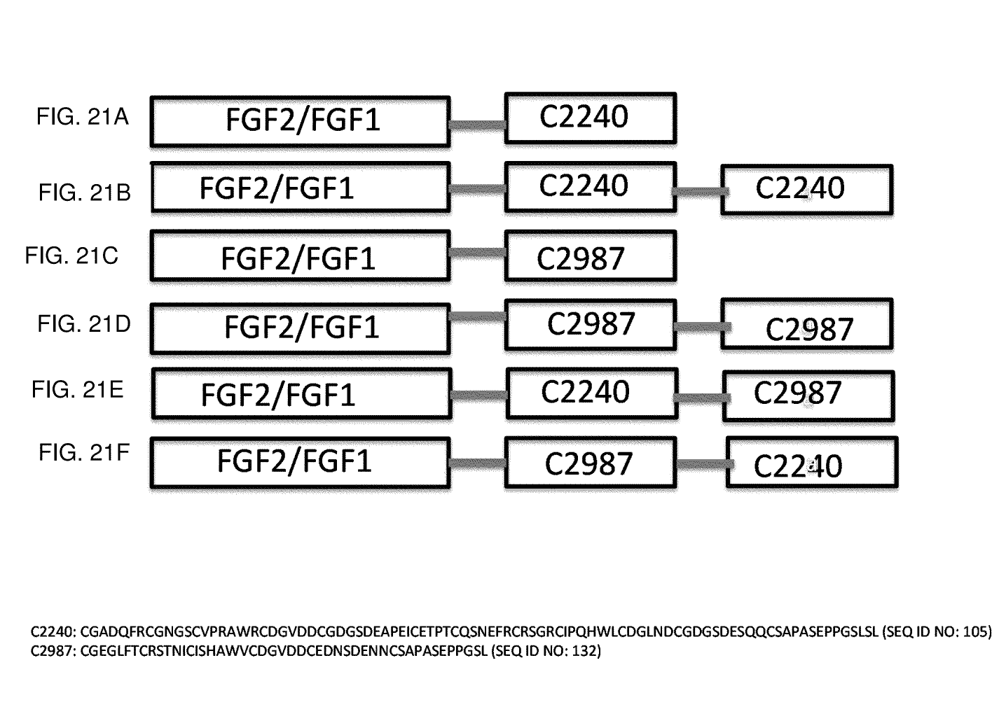

SEQ ID NO: 156 provides an exemplary FGF2/FGF1/β-Klotho binding protein chimera sequence (C2240). This is represented in FIG. 21A.

SEQ ID NO: 157 provides an exemplary FGF2/FGF1/β-Klotho binding protein chimera sequence (C2240) with two β-Klotho binding protein portions. This is represented in FIG. 21B.

SEQ ID NO: 158 provides an exemplary FGF2/FGF1/FGFR1c-binding protein chimera sequence (C2987). This is represented in FIG. 21C.

SEQ ID NO: 159 provides an exemplary FGF2/FGF1/FGFR1c binding protein chimera sequence (C2987) with two FGFR1c binding protein portions. This is represented in FIG. 21D.

SEQ ID NO: 160 provides an exemplary FGF2/FGF1/β-Klotho binding protein/FGFR1c-binding protein chimera sequence. This is represented in FIG. 21E.

SEQ ID NO: 161 provides an exemplary FGF2/FGF1/FGFR1c-binding protein/β-Klotho-binding protein chimera sequence. This is represented in FIG. 21F.

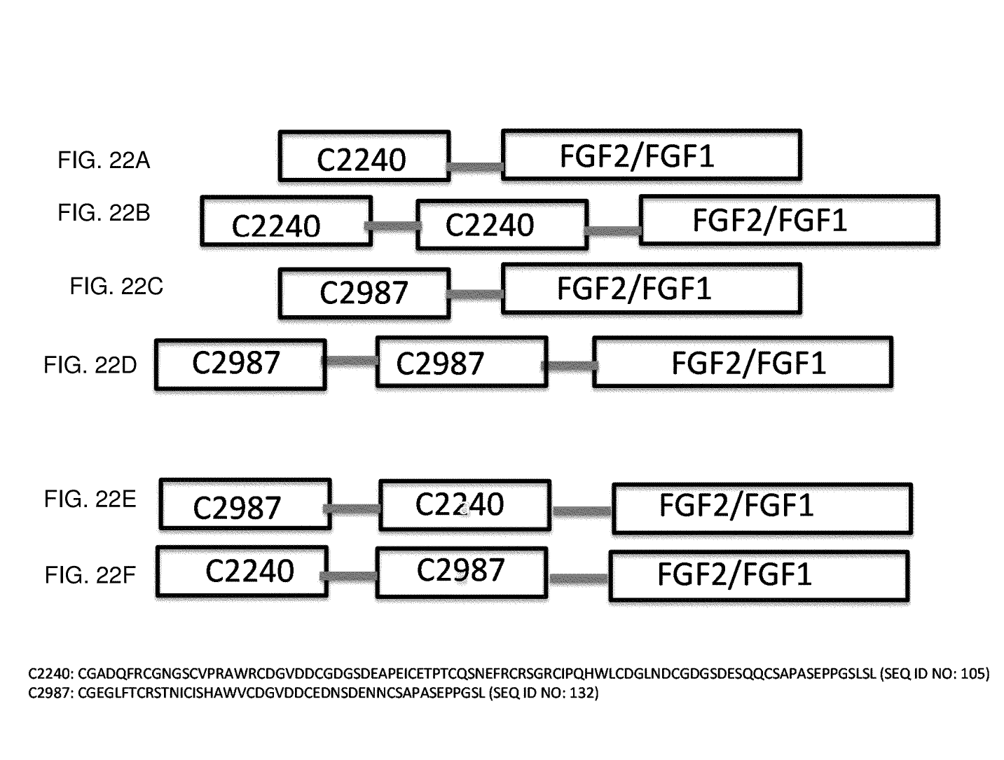

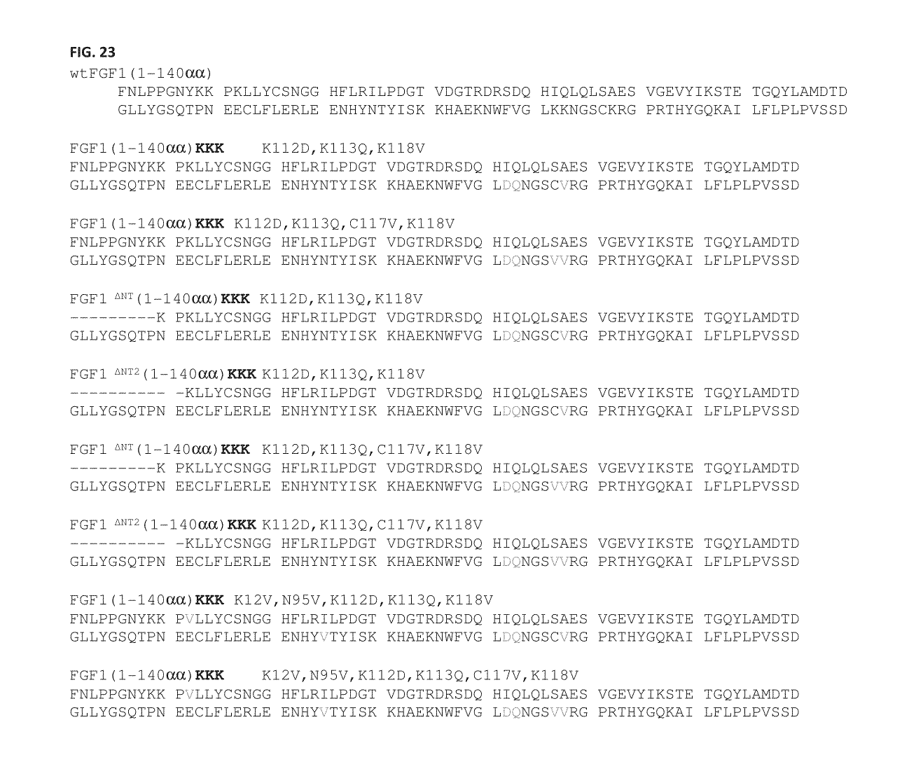

SEQ ID NO: 162 provides an exemplary FGF2/FGF1/β-Klotho binding protein chimera sequence (C2240). This is represented in FIG. 22A.

SEQ ID NO: 163 provides an exemplary FGF2/FGF1/β-Klotho binding protein chimera sequence (C2240) with two β-Klotho binding protein portions. This is represented in FIG. 22B.

SEQ ID NO: 164 provides an exemplary FGF2/FGF1/FGFR1c-binding protein chimera sequence (C2987). This is represented in FIG. 22C.

SEQ ID NO: 165 provides an exemplary FGF2/FGF1/FGFR1c binding protein chimera sequence (C2987) with two FGFR1c-binding protein portions. This is represented in FIG. 22D.

SEQ ID NO: 166 provides an exemplary FGF2/FGF1/β-Klotho binding protein/FGFR1c-binding protein chimera sequence. This is represented in FIG. 22E.

SEQ ID NO: 167 provides an exemplary FGF2/FGF1/FGFR1c-binding protein/β-Klotho binding protein chimera sequence. This is represented in FIG. 22F.

SEQ ID NO: 168 provides an exemplary FGF1 heparan binding KKK mutant analog K112D, K113Q, K118V (wherein numbering refers to SEQ ID NO: 14).

SEQ ID NO: 169 provides an exemplary FGF1 heparan binding KKK mutant analog with mutations K112D, K113Q, C117V, K118V (wherein numbering refers to SEQ ID NO: 14).

SEQ ID NO: 170 provides an exemplary FGF1 heparan binding KKK mutant analog with an N-terminal truncation and mutations K112D, K113Q, K118V (wherein numbering refers to SEQ ID NO: 14).

SEQ ID NO: 171 provides an exemplary FGF1 heparan binding KKK mutant analog with an N-terminal truncation and mutations K112D, K113Q, K118V (wherein numbering refers to SEQ ID NO: 14).

SEQ ID NO: 172 provides an exemplary FGF1 heparan binding KKK mutant analog with an N-terminal truncation and mutations K112D, K113Q, C117V, K118V (wherein numbering refers to SEQ ID NO: 14).

SEQ ID NO: 173 provides an exemplary FGF1 heparan binding KKK mutant analog with an N-terminal truncation and mutations K112D, K113Q, C117V, K118V (wherein numbering refers to SEQ ID NO: 14).

SEQ ID NO: 174 provides an exemplary FGF1 heparan binding KKK mutant analog with mutations K12V, N95V, K112D, K113Q, K118V (wherein numbering refers to SEQ ID NO: 14.

SEQ ID NO: 175 provides an exemplary FGF1 heparan binding KKK mutant analog with mutations K12V, N95V, K112D, K113Q, C117V, K118V (wherein numbering refers to SEQ ID NO: 14).

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Oct. 21, 2013. All references and GenBank® Accession numbers cited herein are incorporated by reference.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration:

To provide or give a subject an agent, such as an FGF2/FGF1 chimeric protein or nucleic acid molecule, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes.

Beta-Klotho Binding Domain or Protein:

A peptide sequence that binds selectively to β-Klotho (such as human β-Klotho, OMIM 61135, GenBank® Accession No. NP_783864.1), but not to other proteins. β-Klotho is a cofactor for FGF21 activity. Such a binding domain can include one or more monomers (wherein the monomers can be the same or different sequences), thereby generating a multimer (such as a dimer). In specific examples, such a domain/protein is not an antibody. Exemplary β-Klotho binding proteins are provided in SEQ ID NO: 105, 106, 107, 108, 109, 110, 111, 112, 113, 114, 115, 116, 117, 118, 119, 120, 121, 122, 123, 124, 125, 126, 127, 128, 129, and 130, as well as U.S. Pat. No. 8,372,952, U.S. Publication No. 2013/0197191, and Smith et al., PLoS One 8:e61432, 2013, all herein incorporated by reference.

A β-Klotho binding protein “specifically binds” to β-Klotho when the dissociation constant (KD) is at least about 1×10⁻⁷ M, at least about 1.5×10⁻⁷, at least about 2×10⁻⁷, at least about 2.5×10⁻⁷, at least about 3×10⁻⁷, at least about at least about 5×10⁻⁷ M, at least about 1×10⁻⁸ M, at least about 5×10⁻⁸, at least about 1×10⁻⁹, at least about 5×10⁻⁹, at least about 1×10⁴⁰, or at least about 5×10⁻¹⁰ M. In one embodiment, KD is measured by a radiolabeled antigen binding assay (RIA) performed with the β-Klotho binding protein and β-Klotho. In another example, K_{D} is measured using an ELISA assay.

C-Terminal Portion:

A region of a protein sequence that includes a contiguous stretch of amino acids that begins at or near the C-terminal residue of the protein. A C-terminal portion of the protein can be defined by a contiguous stretch of amino acids (e.g., a number of amino acid residues).

Chimeric Protein:

A protein that includes at least a portion of the sequence of a full-length first protein (e.g., FGF2) and at least a portion of the sequence of a full-length second protein (e.g., FGF1), where the first and second proteins are different. A chimeric polypeptide also encompasses polypeptides that include two or more non-contiguous portions derived from the same polypeptide. In some examples, a chimeric protein includes an FGF2/FGF1 chimeric protein, along with one or more other peptides, such as a β-Klotho binding protein, an FGF1Rc-binding protein, or both. The two or more different peptides can be joined directly or indirectly, for example using a linker.

Diabetes Mellitus:

A group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 2 diabetes results from insulin resistance, a condition in which cells fail to use insulin properly, sometimes combined with an absolute insulin deficiency. This form is also called “non insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and in some examples diagnosed by demonstrating any one of:
a. Fasting plasma glucose level≧7.0 mmol/1(126 mg/dl);
b. Plasma glucose≧11.1 mmol/1(200 mg/dL) two hours after a 75 g oral glucose load as in a glucose tolerance test;
c. Symptoms of hyperglycemia and casual plasma glucose≧11.1 mmol/1(200 mg/dl);
d. Glycated hemoglobin (Hb A1C)≧6.5%

Effective Amount or Therapeutically Effective Amount:

The amount of agent, such as an FGF2/FGF1 chimeric protein (or nucleic acid encoding such) disclosed herein, that is an amount sufficient to prevent, treat (including prophylaxis), reduce and/or ameliorate the symptoms and/or underlying causes of any of a disorder or disease. In one embodiment, an “effective amount” is sufficient to reduce or eliminate a symptom of a disease, such as a diabetes (such as type II diabetes), for example by lowering blood glucose.

Fibroblast Growth Factor 1 (FGF1):

OMIM 13220. Includes FGF1 nucleic acid molecules and proteins. A protein that binds to the FGF receptor, and is also known as the acidic FGF. FGF1 sequences are publically available, for example from GenBank® sequence database (e.g., Accession Nos. NP_00791 and NP_034327 provide exemplary FGF1 protein sequences, while Accession Nos. NM_000800 and NM_010197 provide exemplary FGF1 nucleic acid sequences). Other examples are provided in SEQ ID NOS: 5-8 and 14. One of ordinary skill in the art can identify additional FGF1 nucleic acid and protein sequences, including FGF1 variants. A mutated FGF1 is a variant of FGF1 (e.g., a variant of any of SEQ ID NOS: 5, 6, 7, 8 or 14, such as one having at least 90%, at least 95%, at least 96%, at least 97%, at least 98% or at least 99% sequence identity to SEQ ID NO: 5, 6, 7, 8 or 14). In one example, such a variant includes an N-terminal truncation, at least one point mutation, or combinations thereof, such as changes that decrease mitogenicity of FGF1. Specific exemplary FGF1 mutant proteins are shown in SEQ ID NOS: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, and 175 and can be part of the FGF2/FGF1 chimeras disclosed herein.

Fibroblast Growth Factor 2 (FGF2):

OMIM 134920. Includes FGF2 nucleic acid molecules and proteins. FGF2 is a paracrine FGF that binds to the FGF receptor, and is also known as the basic FGF. FGF2 is present in basement membranes and in the subendothelial extracellular matrix of blood vessels. It stays membrane-bound as long as there is no signal peptide. FGF2 sequences are publically available, for example from GenBank® sequence database (e.g., Accession Nos. NP_001997 and NP_032032 provide exemplary FGF1 protein sequences, while Accession Nos. NM_002006 and NM_008006 provide exemplary FGF2 nucleic acid sequences). Other examples are provided in SEQ ID NOS: 1-4. One of ordinary skill in the art can identify additional FGF1 nucleic acid and protein sequences, including FGF2 variants. A mutated FGF2 is a variant of FGF2 (e.g., a variant of any of SEQ ID NOS: 1-4, such as one having at least 90%, at least 95%, at least 96%, at least 97%, at least 98% or at least 99% sequence identity to SEQ ID NO: 1, 2, 3, or 4). In one example, such a variant includes an N-terminal truncation, at least one point mutation, or combinations thereof. Specific exemplary FGF2 mutant proteins are shown in SEQ ID NOS: 22, 28, 29, and 30, and can be part of the FGF2/FGF1 chimeras disclosed herein.

Fibroblast Growth Factor Receptor 1c (FGFR1c) Binding Domain or Protein:

A peptide sequence that binds selectively to FGFR1c (such as human FGFR1c, e.g., GenBank® Accession No. NP_001167536.1 or NP_056934.2), but not to other proteins. FGFR1c is a cofactor for FGF21 activity. Such a binding domain can include one or more monomers (wherein the monomers can be the same or different sequences), thereby generating a multimer (such as a dimer). In specific examples, such a domain/protein is not an antibody. Exemplary FGFR1c-binding proteins can be found in SEQ ID NOS: 131, 132, 133, 134, 135, 136, 137, 138, 139, 140, 141, 142, 143, 144, 145, 146, 147, 148, 149, 150 and 151 as well as U.S. Pat. No. 8,372,952, U.S. Publication No. 2013/0197191, and Smith et al., PLoS One 8:e61432, 2013, all herein incorporated by reference.

A FGFR1c binding protein “specifically binds” to FGFR1c when the dissociation constant (KD) is at least about 1×10⁻⁷ M, at least about 1.5×10⁻⁷, at least about 2×10⁻⁷, at least about 2.5×10⁻⁷, at least about 3×10⁻⁷, at least about at least about 5×10⁻⁷ M, at least about 1×10⁻⁸ M, at least about 5×10⁻⁸, at least about 1×10⁻⁹, at least about 5×10⁻⁹, at least about 1×10⁴⁰, or at least about 5×10⁻¹⁰ M. In one embodiment, KD is measured by a radiolabeled antigen binding assay (RIA) performed with the FGFR1c-binding protein and FGFR1c. In another example, KD is measured using an ELISA assay.

Fibroblast Growth Factor Receptor 1c (FGFR1c):

Also known as FGFR1 isoform 2. Includes FGFR1c nucleic acid molecules and proteins. FGFR1c and β-Klotho can associate with FGF21 to form a signaling complex. FGFR1c sequences are publically available, for example from the GenBank® sequence database (e.g., Accession Nos. NP_001167536.1 and NP_056934.2 provide exemplary FGFR1c protein sequences). One of ordinary skill in the art can identify additional FGFR1c nucleic acid and protein sequences, including FGFR1c variants.

Host Cells:

Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. Thus, host cells can be transgenic, in that they include nucleic acid molecules that have been introduced into the cell, such as a nucleic acid molecule encoding a chimeric protein disclosed herein.

Isolated:

An “isolated” biological component (such as a nucleic acid molecule or chimeric protein) has been substantially separated, produced apart from, or purified away from other biological components in the cell of the organism in which the component naturally occurs, such as other chromosomal and extrachromosomal DNA and RNA, and proteins. Nucleic acids molecules and chimeric proteins which have been “isolated” thus include nucleic acids and proteins purified by standard purification methods. The term also embraces nucleic acid molecules and chimeric proteins prepared by recombinant expression in a host cell as well as chemically synthesized nucleic acids. A purified cell, chimeric protein, or nucleic acid molecule can be at least 90%, at least 95%, at least 96%, at least 97%, at least 98%, or at least 99% pure.

Linker:

A moiety or group of moieties that joins or connects two or more discrete separate peptide or proteins, such as monomer domains, for example to generate a chimeric protein. In one example a linker is a substantially linear moiety. Exemplary linkers that can be used to generate the chimeric proteins provided herein include but are not limited to: peptides, nucleic acid molecules, peptide nucleic acids, and optionally substituted alkylene moieties that have one or more oxygen atoms incorporated in the carbon backbone. A linker can be a portion of a native sequence, a variant thereof, or a synthetic sequence. Linkers can include naturally occurring amino acids, non-naturally occurring amino acids, or a combination of both. In one example a linker is composed of at least 5, at least 10, at least 15 or at least 20 amino acids, such as 5 to 10, 5 to 20, or 5 to 50 amino acids. In one example the linker is a poly alanine.

Mammal: This term includes both human and non-human mammals. Similarly, the term “subject” includes both human and veterinary subjects (such as cats, dogs, cows, and pigs).

Metabolic disorder/disease: A disease or disorder that results from the disruption of the normal mammalian process of metabolism. Includes metabolic syndrome.

Examples include but are not limited to: (1) glucose utilization disorders and the sequelae associated therewith, including diabetes mellitus (Type I and Type-2), gestational diabetes, hyperglycemia, insulin resistance, abnormal glucose metabolism, “pre-diabetes” (Impaired Fasting Glucose (IFG) or Impaired Glucose Tolerance (IGT)), and other physiological disorders associated with, or that result from, the hyperglycemic condition, including, for example, histopathological changes such as pancreatic β-cell destruction; (2) dyslipidemias and their sequelae such as, for example, atherosclerosis, coronary artery disease, cerebrovascular disorders and the like; (3) other conditions which may be associated with the metabolic syndrome, such as obesity and elevated body mass (including the co-morbid conditions thereof such as, but not limited to, nonalcoholic fatty liver disease (NAFLD), nonalcoholic steatohepatitis (NASH), and polycystic ovarian syndrome (PCOS)), and also include thromboses, hypercoagulable and prothrombotic states (arterial and venous), hypertension, cardiovascular disease, stroke and heart failure; (4) disorders or conditions in which inflammatory reactions are involved, including atherosclerosis, chronic inflammatory bowel diseases (e.g., Crohn's disease and ulcerative colitis), asthma, lupus erythematosus, arthritis, or other inflammatory rheumatic disorders; (5) disorders of cell cycle or cell differentiation processes such as adipose cell tumors, lipomatous carcinomas including, for example, liposarcomas, solid tumors, and neoplasms; (6) neurodegenerative diseases and/or demyelinating disorders of the central and peripheral nervous systems and/or neurological diseases involving neuroinfiammatory processes and/or other peripheral neuropathies, including Alzheimer's disease, multiple sclerosis, Parkinson's disease, progressive multifocal leukoencephalopathy and Guillian-Barre syndrome; (7) skin and dermatological disorders and/or disorders of wound healing processes, including erythemato-squamous dermatoses; and (8) other disorders such as syndrome X, osteoarthritis, and acute respiratory distress syndrome. Other examples are provided in WO 2014/085365 (herein incorporated by reference).

In specific examples, the metabolic disease includes one or more of (such as at least 2 or at least 3 of): diabetes (such as type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), and cardiovascular diseases (e.g., hypertension).

N-Terminal Portion:

A region of a protein sequence that includes a contiguous stretch of amino acids that begins at or near the N-terminal residue of the protein. An N-terminal portion of the protein can be defined by a contiguous stretch of amino acids (e.g., a number of amino acid residues).

Operably Linked:

A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as an FGF2/FGF1 chimeric coding sequence). Generally, operably linked DNA sequences are contiguous and, where necessary to join two protein coding regions (such as regions of FGF1 and FGF2), in the same reading frame.

Pharmaceutically Acceptable Carriers:

The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the disclosed FGF2/FGF1 chimeric proteins (or nucleic acid encoding such) herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter:

Ann array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant:

A recombinant nucleic acid molecule is one that has a sequence that is not naturally occurring or has a sequence that is made by an artificial combination of two otherwise separated segments of sequence (e.g., a FGF2/FGF1 chimera). This artificial combination can be accomplished by routine methods, such as chemical synthesis or by the artificial manipulation of isolated segments of nucleic acids, such as by genetic engineering techniques. Similarly, a recombinant protein is one encoded for by a recombinant nucleic acid molecule. Similarly, a recombinant or transgenic cell is one that contains a recombinant nucleic acid molecule and expresses a recombinant protein.

Sequence Identity of Amino Acid Sequences:

The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of the chimeric proteins disclosed herein are typically characterized by possession of at least about 95%, 96%, 97%, 98% or 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided. Thus, a portion of an FGF1 or FGF2 protein disclosed herein used to make a chimeric FGF2/FGF1 protein can have at least 80%, at least 85%, at least 90%, at least 92%, at least 95%, at least 98%, at least 99% sequence identity to such disclosed FGF1 fragments (e.g., SEQ ID NO: 14, 15, 16, 17, 18, 19, 20, 21, 27, 37, 38, 39, 40, 41, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97 and 98) and FGF2 fragments (e.g., amino acids 1 to 21 or 10-21 of SEQ ID NO: 2 or SEQ ID NO: 28). In addition, exemplary chimeric FGF2/FGF1 proteins have at least 80%, at least 85%, at least 90%, at least 92%, at least 95%, at least 98%, or at least 99% sequence identity to SEQ ID NO: 9, 10, 11, 12, 13, 99, 100, 101, 102, 103, or 104 and retain the ability to lower blood glucose in a mammal.

Similarly, exemplary β-Klotho binding domain sequences that can be used in the FGF2/FGF1 chimeras disclosed herein in some examples have at least 70%, at least 80%, at least 85%, at least 90%, at least 92%, at least 95%, at least 97%, at least 98%, or at least 99% sequence identity to SEQ ID NO: 105, 106, 107, 108, 109, 110, 111, 112, 113, 114, 115, 116, 117, 118, 119, 120, 121, 122, 123, 124, 125, 126, 127, 128, 129, 130, 152, 153, 154, or 155.

Similarly, exemplary FGFR1c binding sequences that can be used in the mutant FGF1 chimeras disclosed herein in some examples have at least 70%, at least 80%, at least 85%, at least 90%, at least 92%, at least 95%, at least 97%, at least 98%, or at least 99% sequence identity to SEQ ID NO: 131, 132, 133, 134, 135, 136, 137, 138, 139, 140, 141, 142, 143, 144, 145, 146, 147, 148, 149, 150, 151, 152, 153, 154, or 155 and retain the ability to lower blood glucose in a mammal.

Similarly, exemplary chimeric FGF2/FGF1 coding sequences have at least 70%, at least 80%, at least 85%, at least 90%, at least 92%, at least 95%, at least 98%, or at least 99% sequence identity to SEQ ID NO: 31, 32, or 33 and retain the ability to encode a protein that can lower blood glucose in a mammal.

Similarly, exemplary chimeric FGF2/FGF1 sequences with a β-Klotho binding portion and/or a FGFR1c-binding portion have at least 70%, at least 80%, at least 85%, at least 90%, at least 92%, at least 95%, at least 98%, or at least 99% sequence identity to SEQ ID NO: 156, 157, 158, 159, 160, 161, 162, 163, 164, 165, 166 or 167 and retain the ability to lower blood glucose in a mammal.

Subject:

Any mammal, such as humans, non-human primates, pigs, sheep, cows, dogs, cats, rodents and the like which is to be the recipient of the particular treatment, such as treatment with a chimeric protein (or nucleic acid encoding such) provided herein. In two non-limiting examples, a subject is a human subject or a murine subject. In some examples, the subject has one or more metabolic diseases, such as diabetes (e.g., type 2 diabetes, non-type 2 diabetes, type 1 diabetes, latent autoimmune diabetes (LAD), or maturity onset diabetes of the young (MODY)), polycystic ovary syndrome (PCOS), metabolic syndrome (MetS), obesity, non-alcoholic steatohepatitis (NASH), non-alcoholic fatty liver disease (NAFLD), dyslipidemia (e.g., hyperlipidemia), cardiovascular disease (e.g., hypertension), or combinations thereof. In some examples, the subject has elevated blood glucose.

Transduced and Transformed:

A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses {Wolff, J. A., ed, Gene Therapeutics, Birkhauser, Boston, USA (1994)}. In the case of infection by retroviruses, the infecting retrovirus particles are absorbed by the target cells, resulting in reverse transcription of the retroviral RNA genome and integration of the resulting provirus into the cellular DNA.

Transgene:

An exogenous gene supplied by a vector. In one example, a transgene includes an FGF2/FGF1 chimeric coding sequence.

Vector:

A nucleic acid molecule as introduced into a host cell, thereby producing a transformed host cell. A vector may include nucleic acid sequences that permit it to replicate in the host cell, such as an origin of replication. A vector may also include one or more FGF2/FGF1 chimeric coding sequences and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### Overview

Provided herein are chimeric proteins that include an N-terminus coupled to a C-terminus, wherein the N-terminus comprises an N-terminal portion of fibroblast growth factor (FGF) 2 protein and the C-terminus comprises a portion of an FGF1 protein. Such proteins are referred to herein as FGF2/FGF1 chimeric proteins, or FGF24 proteins. In some examples, the N-terminus of the FGF2/FGF1 chimeric protein includes at least 12 consecutive amino acids from amino acids 1-30 of FGF2, such as at least 12 consecutive amino acids from amino acids 1-30 of SEQ ID NO: 2 or 4. In some examples, the C-terminus of the FGF2/FGF1 chimeric protein includes at least 120 or at least 130 consecutive amino acids from amino acids 5-141 of FGF1, such as at least 120 consecutive amino acids from amino acids 5-141 of SEQ ID NO: 6 or 8 or at least 120 consecutive amino acids from SEQ ID NO: 14.

In some examples, the chimeric protein comprises at least 80% sequence identity to SEQ ID NOS 9, 10, 11, 12, 13, 99, 100, 101, 102, 103, or 104 and retains the ability to lower blood glucose in a mammal. Thus, the chimeric protein can have at least 90%, at least 95%, at least 96%, at least 97%, at least 98% or at least 99% sequence identity to SEQ ID NO: 9, 10, 11, 12, 13, 99, 100, 101, 102, 103, or 104 and has the ability to lower blood glucose in a mammal. In some examples, the FGF2/FGF1 chimeric protein includes or consists of SEQ ID NO: 9, 10, 11, 12, 13, 99, 100, 101, 102, 103, or 104. The disclosure encompasses variants of the disclosed FGF2/FGF1 chimeric proteins, such as SEQ ID NO: 9, 10, 11, 12, 13, 99, 100, 101, 102, 103, or 104 having at least 1, at least 2, at least 3, at least 4 at least 5, at least 10, at least 15, or at least 20 mutations, such as 1 to 4, 1 to 5, 1 to 8, or 1 to 10 mutations, such as 1, 2, 3, 4, 5, 6, 7, 8, 9 or 10 mutations, such as conservative amino acid substitutions or substitutions shown in Table 1, 2, and/or 3, and retains the ability to lower blood glucose in a mammal.

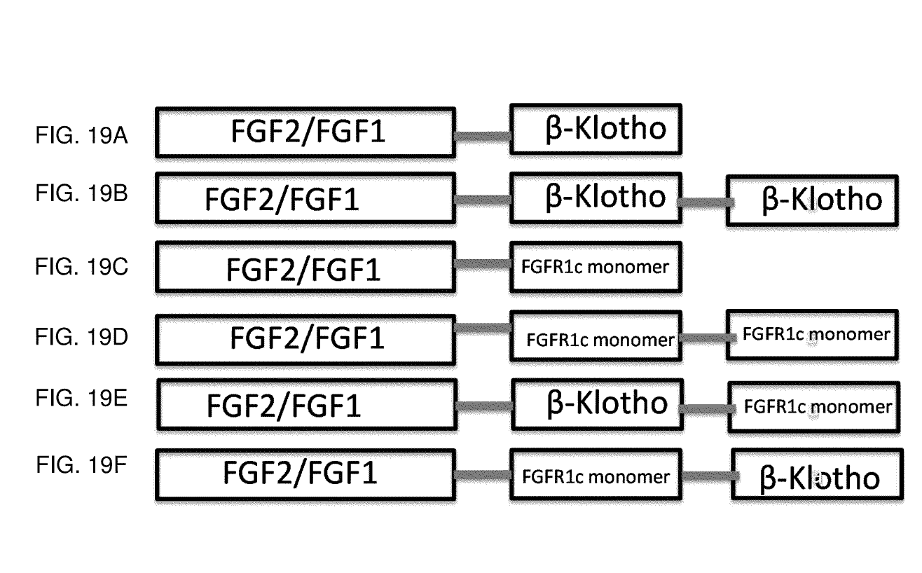

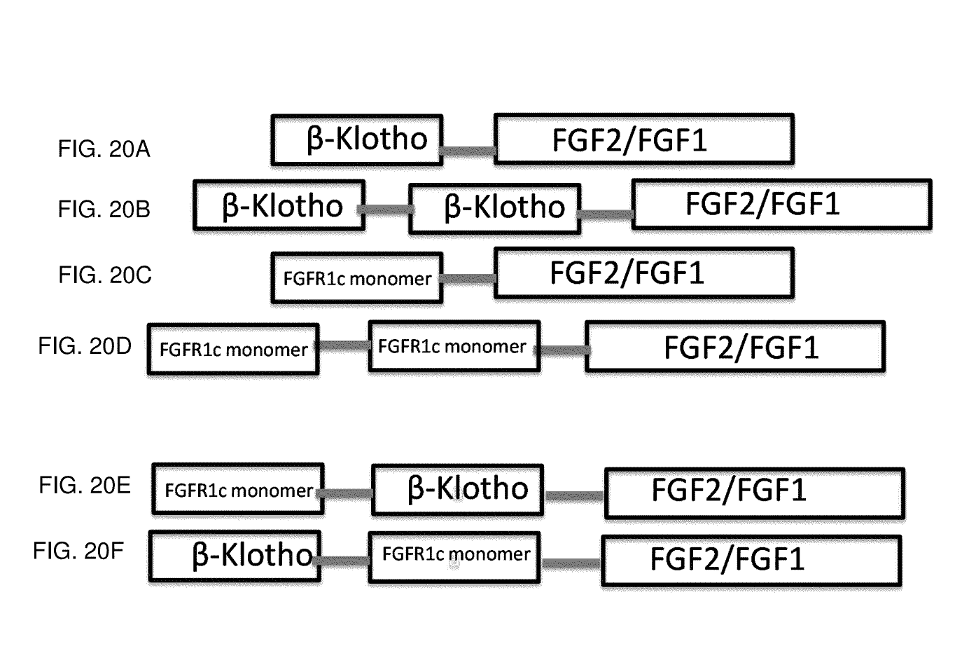

In some examples, the FGF2/FGF1 chimera includes additional sequences at its N- and/or C-terminus. In one example the FGF2/FGF1 chimera includes at its N- and/or C-terminus at least 10, at least 20, at least 30, at least 35, at least 40, at least 50, at least 60, at least 70, at least 80, at least 90, at least 100, at least 120, at least 150, at least 180, or at least 200 amino acids (such as 20-500, 20 to 250, 30 to 200, 35 to 180, 37 to 90, or 37 to 180 amino acids) of a protein that selectively binds to β-Klotho, such as SEQ ID NO: 105, 106, 107, 108, 109, 110, 111, 112, 113, 114, 115, 116, 117, 118, 119, 120, 121, 122, 123, 124, 125, 126, 127, 128, 129, or 130, wherein the chimera retains the ability to lower blood glucose in a mammal. Thus, for example, one end of the FGF2/FGF1 chimera can be joined directly to the end of a β-Klotho-binding protein. This embodiment is schematically shown in FIGS. 19A, 19B, 20A, 20B, 21A, 21B, 22A, and 22B. The β-Klotho binding peptide can be at the N-terminus (e.g., FIG. 20A) or the C-terminus (e.g., FIG. 19A) of the FGF2/FGF1 chimera. In addition, more than one β-Klotho binding peptide can be present (e.g., FIGS. 19B, 20B, 21B, and 22B). Examples of β-Klotho-binding proteins that can be used are shown in SEQ ID NOS: 105, 106, 107, 108, 109, 110, 111, 112, 113, 114, 115, 116, 117, 118, 119, 120, 121, 122, 123, 124, 125, 126, 127, 128, 129, and 130. In some examples, the FGF2/FGF1 chimera and β-Klotho-binding protein portion are linked indirectly through the use of a linker, such as one composed of at least 5, at least 10, at least 15 or at least 20 amino acids. In one example the linker is a polyalanine.

In some examples, the FGF2/FGF1 chimera includes additional sequences at its N- and/or C-terminus. In one example the FGF2/FGF1 chimera includes at least 10, at least 20, at least 30, at least 35, at least 40, at least 50, at least 60, at least 70, at least 80, at least 90, at least 100, at least 120, at least 150, at least 180, or at least 200 amino acids (such as 20-500, 20 to 250, 30 to 200, 35 to 180, 37 to 90, or 37 to 180 amino acids) of a protein that selectively binds to FGFR1c, such as any of SEQ ID NOS: 131, 132, 133, 134, 135, 136, 137, 138, 139, 140, 141, 142, 143, 144, 145, 146, 147, 148, 149, 150 and 151, wherein the chimera retains the ability to lower blood glucose in a mammal. Thus, for example, one end of the FGF2/FGF1 chimera can be joined directly to the end of an FGFR1c-binding protein. This embodiment is schematically shown in FIGS. 19C, 19D, 20C, 20D, 21C, 21D, 22C, and 22D. The FGFR1c binding peptide can be at the N-terminus (e.g., FIG. 20C) or the C-terminus (e.g., FIG. 19C) of the FGF2/FGF1 chimera. In addition, more than one FGFR1c binding peptide can be present (e.g., FIGS. 19D, 20D, 21D, and 22D). Examples of FGFR1c-binding proteins that can be used are shown in SEQ ID NOS: 131, 132, 133, 134, 135, 136, 137, 138, 139, 140, 141, 142, 143, 144, 145, 146, 147, 148, 149, 150 and 151. In some examples, the FGF2/FGF1 chimera and FGFR1c-binding protein portion are linked indirectly through the use of a linker, such as one composed of at least 5, at least 10, at least 15 or at least 20 amino acids. In one example the linker is a polyalanine.

In some examples, the FGF2/FGF1 chimera includes additional sequences at its N- and/or C-terminus. In one example the FGF2/FGF1 chimera includes at least 10, at least 20, at least 30, at least 35, at least 40, at least 50, at least 60, at least 70, at least 80, at least 90, at least 100, at least 120, at least 150, at least 180, or at least 200 amino acids (such as 20-500, 20 to 250, 30 to 200, 35 to 180, 37 to 90, or 37 to 180 amino acids) of a protein that selectively binds to β-Klotho and/or includes at least 10, at least 20, at least 30, at least 35, at least 40, at least 50, at least 60, at least 70, at least 80, at least 90, at least 100, at least 120, at least 150, at least 180, or at least 200 amino acids (such as 20-500, 20 to 250, 30 to 200, 35 to 180, 37 to 90, or 37 to 180 amino acids) of a protein that selectively binds to FGFR1c, such as any of SEQ ID NOS: 160, 161, 166, or 167). Thus, for example, one end of the FGF2/FGF1 chimera can be joined directly to the end of a FGFR1c-binding protein and/or a β-Klotho-binding protein. This embodiment is schematically shown in FIGS. 19E, 19F, 20E, 20F, 21E, 21F, 22E, and 22F. The β-Klotho- and FGFR1c-binding peptides can be at the N-terminus (e.g., FIGS. 20E, 20F, 22E and 22F) or the C-terminus (e.g., FIGS. 19E, 19F, 21E and 21F) of the FGF2/FGF1 chimera. Although not shown, more than one β-Klotho-binding peptide and/or FGFR1c-binding peptide can be present. Although not shown, the β-Klotho-binding peptide and/or FGFR1c-binding peptide can flank the FGF2/FGF1 chimera (e.g., β-Klotho-binding peptide and/or FGFR1c-binding peptide on the N-terminal end of an FGF2/FGF1 chimera, and β-Klotho-binding peptide and/or FGFR1c-binding peptide on the C-terminal end of an FGF2/FGF1 chimera). Examples of β-Klotho-binding peptides that can be used are shown in SEQ ID NO: 105, 106, 107, 108, 109, 110, 111, 112, 113, 114, 115, 116, 117, 118, 119, 120, 121, 122, 123, 124, 125, 126, 127, 128, 129, or 130 and examples of FGFR1c-binding proteins that can be used are shown in SEQ ID NOS: 131, 132, 133, 134, 135, 136, 137, 138, 139, 140, 141, 142, 143, 144, 145, 146, 147, 148, 149, 150 and 151. Examples of β-Klotho-binding peptides/FGFR1c-binding proteins chimeras that can be used are shown in SEQ ID NOS: 152, 153, 154, and 155. In some examples, the FGF2/FGF1 chimera and the FGFR1c-binding protein portion and/or the β-Klotho-binding protein portion are linked indirectly through the use of a linker, such as one composed of at least 5, at least 10, at least 15 or at least 20 amino acids. In one example the linker is a polyalanine.

In some examples, the FGF2/FGF1 chimeric protein that also includes a β-Klotho-binding peptide and/or FGFR1c-binding peptide has at least 80% sequence identity SEQ ID NO: 156, 157, 158, 159, 160, 161, 162, 163, 164, 165, 166 or 167 and retains the ability to lower glucose in a mammal. Thus, the protein can have at least 90%, at least 95%, at least 96%, at least 97%, at least 98% or at least 99% sequence identity to SEQ ID NO: 156, 157, 158, 159, 160, 161, 162, 163, 164, 165, 166 or 167 and has the ability to lower glucose in a mammal. In some examples, the FGF2/FGF1 chimeric protein that also includes a β-Klotho-binding peptide and/or FGFR1c-binding peptide includes or consists of SEQ ID NO: 156, 157, 158, 159, 160, 161, 162, 163, 164, 165, 166 or 167. The disclosure encompasses variants of the disclosed FGF2/FGF1 chimeric proteins that also includes a β-Klotho-binding peptide and/or FGFR1c-binding peptide, such as SEQ ID NO: 156, 157, 158, 159, 160, 161, 162, 163, 164, 165, 166 or 167 having at least 1, at least 2, at least 3, at least 4 at least 5, at least 10, at least 15, or at least 20 mutations, such as 1 to 4, 1 to 5, 1 to 8, or 1 to 10 mutations, such as 1, 2, 3, 4, 5, 6, 7, 8, 9 or 10 mutations, such as conservative amino acid substitutions or substitutions shown in Table 1, 2, and/or 3.

Also provided are isolated nucleic acid molecules encoding the disclosed FGF2/FGF1 chimeric proteins (including those that have a β-Klotho-binding peptide and/or FGFR1c-binding peptide), such as a nucleic acid molecule encoding a protein having at least 80%, at least 85%, at least 90%, at least 95%, at least 96%, at least 97%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167 and encodes a protein that can lower glucose in a mammal Vectors and cells that include such nucleic acid molecules are also provided. For example, such nucleic acid molecules can be expressed in a host cell, such as a bacterium or yeast cell (e.g., E. coli), thereby permitting expression of the FGF2/FGF1 chimeric protein. The resulting FGF2/FGF1 chimeric protein can be purified from the cell.

Methods of using the disclosed FGF2/FGF1 chimeric proteins (or nucleic acid molecules encoding such), including those that further include a β-Klotho-binding peptide and/or FGFR1c-binding peptide, are provided. For example, such methods include administering a therapeutically effective amount of a disclosed FGF2/FGF1 chimeric protein (such as at least 0.5 mg/kg) (or nucleic acid molecules encoding such) to reduce blood glucose in a mammal, such as a decrease of at least 5%. Such FGF2/FGF1 chimeric proteins (such as at least 0.5 mg/kg) (or nucleic acid molecules encoding such) can be used alone or in combination with other agents, such as other glucose reducing agents, such as thiazolidinedione.

In one example, the method is a method of treating a metabolic disease (such as metabolic syndrome, diabetes, obesity, or combinations thereof) in a mammal. Such a method can include administering a therapeutically effective amount of a disclosed FGF2/FGF1 chimeric protein, including those that further include a β-Klotho-binding peptide and/or FGFR1c-binding peptide, (such as at least 0.5 mg/kg) (or nucleic acid molecules encoding such) to treat the metabolic disease.

In one example, the method is a method of reducing fed and fasting blood glucose, improving insulin sensitivity and glucose tolerance, reducing systemic chronic inflammation, ameliorating hepatic steatosis in a mammal, reducing triglycerides, decreasing insulin resistance, reducing hyperinsulinemia, increasing glucose tolerance, reducing hyperglycemia, or combinations thereof. Such a method can include administering a therapeutically effective amount of a disclosed FGF2/FGF1 chimeric protein, including those that further include a β-Klotho-binding peptide and/or FGFR1c-binding peptide, (such as at least 0.5 mg/kg) (or nucleic acid molecules encoding such) to reduce fed and fasting blood glucose, improve insulin sensitivity and glucose tolerance, reduce systemic chronic inflammation, ameliorate hepatic steatosis in a mammal, or combinations thereof.

In some examples, the mammal, such as a human, cat or dog, has diabetes. Methods of administration are routine, and can include subcutaneous, intraperitoneal, intramuscular, or intravenous injection.

In some examples, use of the FGF2/FGF1 chimeras disclosed herein, including those that further include a β-Klotho-binding peptide and/or FGFR1c-binding peptide, does not lead to (or significantly reduces, such as a reduction of at least 20%, at least 50%, at least 75%, or at least 90%) the adverse side effects observed with thiazolidinediones (TZDs) therapeutic insulin sensitizers, including weight gain, increased liver steatosis and bone fractures (e.g., reduced affects on bone mineral density, trabecular bone architecture and cortical bone thickness).

Provided are methods of reducing fed and fasting blood glucose, improving insulin sensitivity and glucose tolerance, reducing systemic chronic inflammation, ameliorating hepatic steatosis, or combinations thereof, in a mammal. Such methods can include administering a therapeutically effective amount of a FGF2/FGF1 chimera disclosed herein, including those that further include a β-Klotho-binding peptide and/or FGFR1c-binding peptide, to the mammal, or a nucleic acid molecule encoding the chimera or a vector comprising the nucleic acid molecule, thereby reducing fed and fasting blood glucose, improving insulin sensitivity and glucose tolerance, reducing systemic chronic inflammation, ameliorating hepatic steatosis, reduce one or more non-HDL lipid levels, or combinations thereof, in a mammal. In some examples, the fed and fasting blood glucose is reduced in the treated subject by at least 10%, at least 20%, at least 30%, at least 50%, at least 75%, or at least 90% as compared to an absence of administration of the FGF2/FGF1 chimera. In some examples, insulin sensitivity and glucose tolerance is increased in the treated subject by at least 10%, at least 20%, at least 30%, at least 50%, at least 75%, or at least 90% as compared to an absence of administration of the FGF2/FGF1 chimera. In some examples, systemic chronic inflammation is reduced in the treated subject by at least 10%, at least 20%, at least 30%, at least 50%, at least 75%, or at least 90% as compared to an absence of administration of FGF2/FGF1 chimera. In some examples, hepatic steatosis is reduced in the treated subject by at least 10%, at least 20%, at least 30%, at least 50%, at least 75%, or at least 90% as compared to an absence of administration of the FGF2/FGF1 chimera. In some examples, one or more lipids (such as a non-HDL, for example IDL, LDL and/or VLDL) are reduced in the treated subject by at least 10%, at least 20%, at least 30%, at least 50%, at least 75%, or at least 90% as compared to an absence of administration of the FGF2/FGF1 chimera. In some examples, triglyceride and or cholesterol levels are reduced with the FGF2/FGF1 chimera by at least 10%, at least 20%, at least 30%, at least 50%, at least 75%, or at least 90% as compared to an absence of administration of the FGF2/FGF1 chimera. In some examples, combinations of these reductions are achieved.

### FGF2/FGF1 Chimeric Proteins and Variants Thereof

The present disclosure provides chimeric proteins that include an N-terminal and a C-terminal portion, wherein the N-terminal portion includes an N-terminal portion of FGF2, while the C-terminal portion includes a portion of FGF1. In some examples, the chimeric FGF2/FGF1 protein is referred to as an FGF24 protein. FGFs are a family of growth factors, with members involved in angiogenesis, wound healing, embryonic development and various endocrine signaling pathways. The FGFs are heparan-binding proteins and interact with cell-surface-associated heparan sulfate proteoglycans. There are currently 22 FGF family members.

In some examples, the N-terminus of the FGF2/FGF1 chimeric protein includes at least 12 consecutive amino acids from amino acids 1-30 of FGF2, such as at least 12, at least 13, at least 14, at least 15, at least 16, at least 17, at least 18, at least 19, at least 20, at least 21, at least 22, at least 23, at least 24, or at least 25 consecutive amino acids from amino acids 1-30 of SEQ ID NO: 2 or 4 (such as 12, 13, 14, 15, 16, 17, 18, 19, 20, 21, 22, 23, 24, 35, 36, 27, 28, 29, 30, 31, 32, 33, 34 35, consecutive amino acids from amino acids 1-35 of SEQ ID NO: 2 or 4, for example 12-30, 12-15, 12-21 or 12-20 consecutive amino acids from amino acids 1-30 of SEQ ID NO: 2 or 4). Examples of least 12 consecutive amino acids from amino acids 1-30 of FGF2 that can be used to generate an FGF2/FGF1 chimeric protein include but are not limited to: MAAGSITTLP ALPEDGGSGA F (amino acids 1 to 21 of SEQ ID NO: 2), MAASGITSLP ALPEDGGAAF (amino acids 1 to 20 of SEQ ID NO: 4), PALPEDGGSGAF (amino acids 10 to 21 of SEQ ID NO: 2), and PALPEDGGAAF (amino acids 10 to 20 of SEQ ID NO: 4). In some examples, such an FGF2 portion of the FGF2/FGF1 chimeric protein includes 1, 2, 3, 4, or 5 substitutions, such as at least one of those shown in Table 3, at least one conservative substitution, or combinations thereof.

In some examples, this contiguous region of FGF2 can be mutated, for example to change receptor binding specificity and/or mitogenicity (e.g., allow the resulting chimeric protein it is used in to bind to all FGF receptors and/or decrease mitogenicity). In some examples, a fragment of FGF2 that includes at least 12 contiguous amino acids from amino acids 1-30 of FGF2 can include 1, 2, 3, 4, or 5 point mutations. Examples of point mutations include amino acid deletions, substitutions, or additions, such as those shown in Table 3. Examples of least 12 consecutive amino acids from amino acids 1-30 of FGF2 containing 1 to 5 point mutations that can be used to generate an FGF2/FGF1 chimeric protein include but are not limited to: MAAGSITTLP ALPEDGGSFA F (amino acids 1 to 21 of SEQ ID NO: 10); MAAGSITTLP ALPEDGGSFNL (amino acids 1 to 21 of SEQ ID NO: 11); PALPEDGGSFAF (amino acids 10 to 21 of SEQ ID NO: 10); PALPEDGGSFNL (amino acids 10 to 21 of SEQ ID NO: 11); MPALPEDGGSGAF (amino acids 1 to 13 of SEQ ID NO: 13); MPALPEDGGAAF (amino acids 1 to 12 of SEQ ID NO: 28); MPALPEDGFAAF (amino acids 1 to 12 of SEQ ID NO: 29) and MPALPEDGFFSGAF (amino acids 1 to 14 of SEQ ID NO: 30).

In some examples, the C-terminus of the FGF2/FGF1 chimeric protein includes at least 120 consecutive amino acids from amino acids 5-141 of FGF1, such as at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, or at least 140 consecutive amino acids from amino acids 5-141 of SEQ ID NO: 6 or 8 (such as 120-130, 120-135, 130-135, 130-140, or 120-140 consecutive amino acids from amino acids 5-141 of SEQ ID NO: 6 or 8), or such as at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, or at least 140 consecutive amino acids from SEQ ID NO: 14 (such as 120-130, 120-135, or 120-140 consecutive amino acids from SEQ ID NO: 14). Examples of least 120 consecutive amino acids from amino acids 5 to 141 of FGF1 that can be used to generate an FGF2/FGF1 chimeric protein include but are not limited to amino acids 4 to 140 of SEQ ID NO: 14 and the protein sequence shown in SEQ ID NO: 14, 15, 16, 17 or 27. In some examples, the FGF1 portion of the chimera is a truncated version of the mature protein (e.g., SEQ ID NO: 14), which can include for example deletion of at least 5, at least 6, at least 9, at least 10, at least 11, at least 12, at least 13, at least 14, at least 15, or at least 20 consecutive N-terminal amino acids, such as the N-terminal 5, 6, 7, 8, 9, 10, 11, 12, or 13 amino acids of mature FGF1 (e.g., SEQ ID NO: 14). In some examples, such an N-terminally deleted FGF1 protein has reduced mitogenic activity as compared to wild-type (e.g., native) mature FGF1 protein.

In some examples, this contiguous region of FGF1 can be mutated, for example to decrease mitogenicity, increase stability, decrease binding affinity for heparin and/or heparan sulfate (compared to the portion of the FGF1 protein without the modification), or combinations thereof. In some examples, a fragment of FGF1 that includes at least 120 or at least 130 contiguous amino acids from amino acids 5-141 of FGF1 can include at least 1, at last 2, at least 3, at least 4, at least 5, or at least 10 point mutations, such as 1 to 20, 1 to 15, 1 to 5, 1 to 4, 2 to 3, or 1 to 10 point mutations, such as 1, 2, 3, 4, 5, 10, 12, 15, or 20 point mutations. Examples of point mutations include amino acid deletions, substitutions, additions, or combinations thereof, such as those shown in Table 2. Examples of least 120 or at least 130 consecutive amino acids from amino acids 5-141 of FGF1 containing 1 to 20 point mutations that can be used to generate an FGF2/FGF1 chimeric protein include but are not limited to the protein sequence shown in SEQ ID NO: 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175.

In some examples, the FGF2/FGF1 chimeric protein has at its N-terminus a methionine. In some examples, the FGF2/FGF1 chimeric protein is at least 120 amino acids in length, such as at least 125, at least 130, at least 135, at least 140, at least 145, at least 150, at least 155, at least 160, or at least 175 amino acids in length, such as 130-160, 132-160, 140-160, 150-160, 130-200, 130-180, 130-170, or 120-160 amino acids in length.

Exemplary FGF2 and FGF1 sequences that can be used to generate an FGF2/FGF1 chimera are shown in Table 1 (other exemplary FGF1 sequences that can be used to generate an FGF2/FGF1 chimera are shown in SEQ ID NOS: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, and 175). One skilled in the art will appreciate that any FGF2 sequence in Table 1 can be combined with any FGF1 sequence in Table 1, to generate a chimera. In addition, mutations can be made to the sequences shown in the Table, such as one or more of the mutations discussed herein, such as those provided in Tables 2 and 3 (such as 1, 2, 3, 4, 5, 6, 7, 8, 9, 10, 11, 12, 13, 14, 15, 16, 17, 18, 19 or 20 of such mutations). In one example, an FGF2 and/or FGF1 portion of an FGF2/FGF1 chimera has at least 90%, at least 91%, at least 92%, at least 93%, at least 94%, at least 95%, at least 96%, at least 97%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167. In some examples, a spacer is introduced between the FGF2 and FGF1 sequence, to produce the chimera.

TABLE 1
Exemplary FGF2 and FGF1 sequences that can be used to generate an
FGF2/FGF1 chimera

| FGF 2 Sequences | FGF1 Sequences |
|---|---|
| MAAGSITTLP ALPEDGGSGA F (amino | PPGNYK KPKLLYCSNG |
| acids 1-21 of SEQ ID NO: 2) | GHFLRILPDG TVDGTRDRSD |
| | QHIQLQLSAE SVGEVYIKST |
| | ETGQYLAMDT DGLLYGSQTP |
| | NEECLFLERL EENHYNTYIS |
| | KKHAEKNWFVGLKKNGSCKR |
| | GPRTHYGQKA ILFLPLPVSSD (aa 4 to |
| | 140 of SEQ ID NO: 14) |
| MAASGITSLP ALPEDGGAAF (aa 1-20 | FNLPPGNYKK PKLLYCSNGG |
| of SEQ ID NO: 2) | HFLRILPDGT VDGTRDRSDQ |
| | HIQLQLSAES VGEVYIKSTE |
| | TGQYLAMDTD GLLYGSQTPN |
| | EECLFLERLE ENHYNTYISK |
| | KHAEKNWFVG LKKNGSCKRG |
| | PRTHYGQKAI LFLPLPVSSD (SEQ ID |
| | NO: 14) |
| PALPEDGGSGAF (aa 10 - 21 of SEQ ID | K PKLLYCSNGG HFLRILPDGT |
| NO: 2) | VDGTRDRSDQ HIQLQLSAES |
| | VGEVYIKSTE TGQYLAMDTD |
| | GLLYGSQTPN EECLFLERLE |
| | ENHYNTYISK KHAEKNWFVG |
| | LKKNGSCKRG PRTHYGQKAI |
| | LFLPLPVSSD (SEQ ID NO: 15) |
| PALPEDGGAAF (aa 10 - 20 of SEQ ID | LYCSNGG HFLRILPDGT |
| NO: 4) | VDGTRDRSDQ HIQLQLSAES |
| | VGEVYIKSTE TGQYLAMDTD |
| | GLLYGSQTPN EECLFLERLE |
| | ENHYNTYISK KHAEKNWFVG |
| | LKKNGSCKRG PRTHYGQKAI |
| | LFLPLPVSSD (SEQ ID NO: 16) |
| MAAGSITTLP ALPEDGGSFA F (aa 1 to | KLLYCSNGG HFLRILPDGT |
| 21 of SEQ ID NO: 10) | VDGTRDRSDQ HIQLQLSAES |
| | VGEVYIKSTE TGQYLAMDTD |
| | GLLYGSQTPN EECLFLERLE |
| | ENHYNTYISK KHAEKNWFVG |
| | LKKNGSCKRG PRTHYGQKAI |
| | LFLPLPVSSD (SEQ ID NO: 17) |
| MAAGSITTLP ALPEDGGSFNL (aa 1 to | FNLPPGNYKK PVLLYCSNGG |
| 21 of SEQ ID NO: 11) | HFLRILPDGT VDGTRDRSDQ |
| | HIQLQLSAES VGEVYIKSTE |
| | TGQYLAMDTD GLLYGSQTPN |
| | EECLFLERLE ENHYVTYISK |
| | KHAEKNWFVG LKKNGSCKRG |
| | PRTHYGQKAI LFLPLPVSSD (SEQ ID |
| | NO: 18) |
| PALPEDGGSFAF (aa 10 to 21 of SEQ ID | FNLPPGNYKK PVLLYCSNGG |
| NO: 10) | HFLRILPDGT VDGTRDRSDQ |
| | HIQLQVSAES VGEVYIKSTE |
| | TGQYLAMDTDGLLYGSQTPN |
| | EECLFLVRLE ENHYVTYISK |
| | KHAEKNWFVG LKKNGSCKRG |
| | PRTHYGQKAI LFLVLPVSSD |
| | (SEQ ID NO: 19) |
| PALPEDGGSFNL (aa 10 to 21 of SEQ ID | NYKK PKLLYCSNGG HFLRILPDGT |
| NO: 11) | VDGTRDRSDQ HIQLQLSAES |
| | VGEVYIKSTE TGQYLAMDTD |
| | GLLYGSQTPN EECLFLERLE |
| | ENHYNTYISK KHAEKNWFVG |
| | LKKNGSCNRG PRTHYGQKAI |
| | LFLPLPVSSD (SEQ ID NO: 20) |
| MPALPEDGGSGAF (aa 1 to 13 of SEQ | NYKK PKLLYCSNGG HFLRILPDGT |
| ID NO: 13) | VDGTRDRSDQ HIQLQLSAES |
| | VGEVYIKSTE TGQYLAMDTD |
| | GLLYGSQTPN EECLFLERLE |
| | ENHYNTYISK KHAEKNWFVG |
| | LKKNGSCERG PRTHYGQKAI |
| | LFLPLPVSSD (SEQ ID NO: 21) |
| MPALPEDGGAAF (aa 1 to 12 of SEQ ID | KPKLLYCSNG GHFLRILPDG |
| NO: 28) | TVDGTRDRSD QHIQLQLSAE |
| | SVGEVYIKST ETGQYLAMDT |
| | DGLLYGSQTP NEECLFLERL |
| | EENHYNTYIS KKHAEKNWFV |
| | GLKKNGSCKR GPRTHYGQKA |
| | ILFLPLPVSSD (SEQ ID NO: 27) |
| MPALPEDGFAAF (amino acids 1 to 12 | |
| of SEQ ID NO: 29) | |
| MPALPEDGFFSGAF (amino acids 1 to | |
| 14 of SEQ ID NO: 30) | |

Exemplary point mutations that can be introduced into the FGF1 and FGF2 portions of the FGF2/FGF1 chimera are provided in Tables 2 and 3, respectively.

TABLE 2
Exemplary point mutations that can be introduced
into the FGF1 portion of the FGF2/FGF1 chimera

| Location of Point Mutation Position | |
|---|---|
| in mature FGF1 SEQ ID NO: 14 | Mutation Citation |
| K9 | K9T |
| K10 | K10T |
| K12 | K12V |
| L14 | L14A |
| Y15 | Y15F, Y15A, Y15V |
| C16 | C16V, C16A, C16T, C16S |
| H21 | H21Y |
| R35 | R35E, R35V |
| Q40 | Q40P |
| L44 | L44F |
| L46 | L46V |
| S47 | S47I |
| E49 | E49Q, E49A |
| Y55 | Y55F, Y55S, Y55A |
| M67 | M67I |
| L73 | L73V |
| C83 | C83T, C83S, C83A C83V |
| E87 | E87V, E87A, E87S, E87T |
| H93 | H93G, H93A |
| Y94 | Y94V, Y94F, Y94A |
| N95 | N95V, N95A, N95S, N95T |
| H102 | H102Y |
| A103 | A103G |
| EKN (104-106) | Δ104-106 |
| F108 | F108Y |
| V109 | V109L |
| L111 | L111I |
| K112 | K112D, K112E, K112Q |
| K113 | K113Q, K113E, K113D |
| C117 | C117V, C117P, C117T, C117S, |
| | C117A |
| K118 | K118N, K118E, K118V |
| R119 | R119G, R119V, R119E |
| GPR (120-122) | Δ120-122 |
| F132 | F132W |
| L133 | L133A, L133S |
| P134 | P134V |
| L135 | L135A, L135S |

TABLE 3
Exemplary point mutations that can be introduced
into the FGF2 portion of the FGF2/FGF1 chimera

| Location of Point Mutation Position | |
|---|---|
| in mature form of FGF2 SEQ ID NO: 2 | Mutation Citation |
| G19 | G19F |
| H25 | H25N |
| F26 | F26V |

In some examples, the FGF2/FGF1 chimera includes an FGF1 portion having mutations at one or more of the following positions of FGF1: K9, K10, K12, L14, Y15, C16, H21, R35, Q40, L44, L46, S47, E49, Y55, M67, L73, C83, L86, E87, H93, Y94, N95, H102, A103, E104, K105, N106, F108, V109, L111, K112, K113, C117, K118, R119, G120, P121, R122, F132, L133, P134, L135, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or all 42 of these positions, such as one or more of K9T, K10T, K12V, L14A, Y15F, Y15A, Y15V, C16V, C16A, C16T, C16S, H21Y, R35E, R35V, Q40P, L44F, L46V, S47I, E49Q, E49A, Y55F, Y55S, Y55A, M67I, L73V, C83T, C83S, C83A C83V, E87V, E87A, E87S, E87T, H93G, H93A, Y94V, Y94F, Y94A, N95V, N95A, N95S, N95T, H102Y, A103G, Δ104-106, F108Y, V109L, L111I, K112D, K112E, K112Q, K113Q, K113E, K113D, C117V, C117P, C117T, C117S, C117A, K118N, K118E, K118V, R119G, R119V, R119E, Δ120-122, F132W, L133A, L133S, P134V, L135A, and L135S, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of these mutations. In one example, K9 and K10 are replaced with DQ (as in the mutated nuclear localization sequence) or with equivalent residues from FGF21 (or another FGF that does not bind to FGFR4) (wherein the numbering refers to SEQ ID NO: 14).

In some examples, the FGF2/FGF1 chimera includes an FGF1 portion having mutations at one or more of the following positions of FGF1: Y15, E87, Y94, and N95 (wherein the numbering refers to SEQ ID NO: 14), such as one or more of Y15F, Y15A, Y15V, E87V, E87A, E87S, E87T, N95V, N95A, N95S, N95T, Y94V, Y94F, and Y94A (such as 1, 2, 3 or 4 of these mutations). For example, E87 or N95 of FGF1 can be replaced with a non-charged amino acid. In addition, Y15 and Y94 of FGF1 can be replaced with an amino acid that destabilizes the hydrophobic interactions.

In some examples, the FGF2/FGF1 chimera includes an FGF1 portion having mutations at one or more of the following positions of FGF1: Y15, C16, E87, H93, Y94, and N95 (wherein the numbering refers to SEQ ID NO: 14), such as one or more of Y15F, Y15A, Y15V, E87V, E87A, E87S, E87T, H93A, N95V, N95A, N95S, N95T, Y94V, Y94F, and Y94A (such as 1, 2, 3, 4, 5, 6, 7, 8, 9 or 10 of these mutations).

In some examples, the FGF2/FGF1 chimera includes an FGF1 portion having mutations at one or more of the following positions of FGF1: C16, C83, and C117 (wherein the numbering refers to SEQ ID NO: 5), such as one or more of C16V, C16A, C16T, C16S, C83T, C83S, C83A C83V, C117V, C117P, C117T, C117S, and C117A (such as 1, 2, or 3 of these mutations).

In some examples, the FGF2/FGF1 chimera includes an FGF1 portion having mutations at one or more of the following positions of FGF1: E87, Y94, and N95 (wherein the numbering refers to SEQ ID NO: 14), such as one or two of E87V, E87A, E87S, E87T, Y94V, Y94F, Y94A, N95V, N95A, N95S, and N95T.

In some examples, the FGF2/FGF1 chimera includes an FGF1 portion having mutations at one or more of the following positions of FGF1: K12, C83, and C117 (wherein the numbering refers to SEQ ID NO: 14), such as one or more of K12V, K12C, C83T, C83S, C83A, C83V, C117V, C117P, C117T, C117S, and C117A (such as 1, 2, or 3 of these mutations, such as K12V, C83T, and C117V).

In some examples, the FGF2/FGF1 chimera includes an FGF1 portion having mutations at one or more of the following positions of K112, K113, C117, K118 (wherein the numbering refers to SEQ ID NO: 14), such as one or more of K112D, K113Q, C117V, K118V (such as 1, 2, 3 or 4 of these mutations). Specific examples are shown in SEQ ID NOS: 168-175, any of which can be used in the FGF1 portion of the FGF2/FGF1 chimera.

In some examples, the FGF2/FGF1 chimera includes an FGF2 portion having mutations at one or more of the following positions of FGF2: G19, H25, and F26 (wherein the numbering refers to SEQ ID NO: 2), such as one or more of G19F, H25N, and F26Y (such as 1, 2, or 3 of these mutations).

Exemplary FGF2/FGF1 chimeric proteins are provided in SEQ ID NOS: 9, 10, 11, 12, 13, 99, 100, 101, 102, 103, 104, 156, 157, 158, 159, 160, 161, 162, 163, 164, 165, 166 and 167. One skilled in the art will recognize that minor variations can be made to these sequences, without adversely affecting the function of the protein. For example, variants of the FGF2/FGF1 chimeric proteins include those having at least 90%, at least 95%, at least 96%, at least 97%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167, but retain the ability to treat a metabolic disease, or decrease blood glucose in a mammal (such as a mammal with type II diabetes). Thus, variants of SEQ ID NO: 9, 10, 11, 12, 13, 99, 100, 101, 102, 103, 104, 156, 157, 158, 159, 160, 161, 162, 163, 164, 165, 166 or 167 retaining at least 90%, at least 95%, at least 96%, at least 97%, at least 98%, or at least 99% sequence identity are of use in the disclosed methods, for example if they can lower blood glucose in a mammal.

### FGF2

For the FGF2 portion of the chimera, the N-terminal portion of FGF2 can be used to determine or control the receptor specificity of the chimera. For example, an N-terminal portion of FGF2 can be mutated to increase the promiscuity of the protein, for example such that it binds to all FGF receptors (e.g., point mutations G9F, H16N and F17Y in SEQ ID NO: 29).

Exemplary wild-type full-length FGF2 proteins are shown in SEQ ID NOS: 2 (human) and 4 (mouse). In some examples, the FGF2 includes SEQ ID NO: 2 or 4, but without the N-terminal methionine (thus resulting in a 154 or 153 amino acid FGF2 protein). In addition, the mature/active form of FGF2 a portion of the N-terminus is removed, such as the N-terminal 5, 6, 7, 8, 9, 10, 11, 12, 13, 14, 15, 16, 17, 18, 19, 20, or 21 amino acids from SEQ ID NO: 2 or 4. Thus, in some examples the active form of wild-type FGF2 comprises or consists of amino acids 9-155 or 9-154 of SEQ ID NO: 2 or 4, respectively. In some examples, the FGF2 used in the FGF2/FGF1 chimera includes an N-terminal truncation, and a methionine added to the N-terminus of the resulting truncation, such as amino acids 1-13 of SEQ ID NO: 13 (wherein such a sequence can be mutated as discussed herein). Thus, the FGF2 portion of the FGF2/FGF1 chimera can include an N-terminal truncation of the full-length protein.

Mutations can be introduced into FGF2, and the mutated FGF2 sequence used in the FGF2/FGF1 chimera. In some examples, multiple types of mutations disclosed herein are made to the FGF2 portion of the FGF2/FGF1 chimera. Although mutations below are noted by a particular amino acid for example in SEQ ID NO: 2 or 28, one skilled in the art will appreciate that the corresponding amino acid can be mutated in any FGF2 sequence. For example, K30 of SEQ ID NO: 2 corresponds to K21 of SEQ ID NO: 28). Specific exemplary mutations are shown in Tables 1 and 3.

In one example, an N-terminal region of FGF2 is used in the FGF2/FGF1 chimera. For example, the first 50, first 40, first 35, first 30, first 25, first 24, first 23, first 22, first 21, or first 20 amino acids of SEQ ID NO: 2 or 4 can be used, such as the first 21 amino acids of SEQ ID NO: 2 or 4 MAAGSITTLPALPEDGGSGAF (e.g., see SEQ ID NO: 9). In some examples, a sequence that includes or consists of PALPEDGGSGAF (amino acids 9-21 of SEQ ID NO: 2) is used in the FGF2 portion of the FGF2/FGF1 chimera. In some examples, PALPEDGGSGAF (amino acids 9-21 of SEQ ID NO: 2) further includes an N-terminal methionine, thus, MPALPEDGGSGAF (amino acids 1-13 of SEQ ID NO: 13). In some examples, the underlined SG in the FGF2 sequences shown above (e.g., amino acids 18-19 of SEQ ID NO: 2) is mutated, for example to AA (e.g., as shown in amino acids 18-19 of SEQ ID NO: 4), SF, or SFNL. Such mutations can be used to introduce flexibility between the FGF2 and FGF1 portions of the chimera.

In one example, mutations are made in the N-terminal region of FGF2 in order to allow the chimera to bind to all FGF receptors, such as mutating G17F, H25N, F26Y (or combinations thereof) of SEQ ID NO: 2 (see SEQ ID NO: 29 and SEQ ID NO: 30). Methods for measuring such activity are known in the art (e.g., see Beenken et al., J. Biol. Chem. 287(5):3067-78, 2012).

### FGF1

For the FGF1 portion of the chimera, the C-terminal portion of FGF1 can be used to determine or control the mitogenicity of the chimera (for example by mutating the nuclear localization sequence (NLS) or the heparan sulfate binding region) and to provide glucose-lowering ability to the chimera. Mutations can also be introduced into a wild-type FGF1 sequence that affects the stability and receptor binding selectivity of the chimera.

Exemplary wild-type full-length FGF1 proteins are shown in SEQ ID NOS: 6 (human) and 8 (mouse). In some examples, the FGF1 includes SEQ ID NO: 6 or 8, but without the N-terminal methionine (thus resulting in a 154 aa FGF1 protein). In addition, in the mature/active form of wild-type FGF1, a portion of the N-terminus is removed, such as the N-terminal 15, 16, 20, or 21 amino acids from SEQ ID NO: 6 or 8. Thus, in some examples the active form of wild-type FGF1 comprises or consists of amino acids 16-155 or 22-155 of SEQ ID NO: 6 or 8 (e.g., see SEQ ID NO: 14). In some examples, the FGF1 used in the FGF2/FGF1 chimera includes SEQ ID NO: 14 with a methionine added to the N-terminus (wherein such a sequence can be mutated as discussed herein). Thus, the FGF1 portion of the FGF2/FGF1 chimera can include an N-terminal truncation of the full-length protein. In some examples, the FGF1 protein is a truncated version of the mature protein (e.g., SEQ ID NO: 14), which can include for example deletion of at least 2, at least 3, at least 4, at least 5, at least 6, at least 9, at least 10, at least 11, at least 12, at least 13, at least 14, at least 15, or at least 20 consecutive N-terminal amino acids. Thus, in some examples, the mutant FGF1 protein is a truncated version of the mature protein (e.g., SEQ ID NO: 5), such a deletion of the N-terminal 2, 3, 4, 5, 6, 7, 8, 9, 10, 11, 12, 13, 14, 15, 16, 17, 18, 19, 20 or 25 amino acids shown in SEQ ID NO: 14. Examples of N-terminally truncated FGF1 proteins are shown in SEQ ID NOS: 38, 39, 40, 41, 46, 47, 48, 49, 50, 51, 52, 53, 58, 59, 60, 61, 62, 63, 64, 65, 71, 72, 73, 74, 75, 76, 77, 78, 79, 80, 85, 86, 87, 88, 89, 90, 91, 92, 93, 94, 95 and 96, as well as in FIG. 6.

Mutations can be introduced into FGF1, and the mutated FGF1 sequence used in the FGF2/FGF1 chimera. In some examples, multiple types of mutations disclosed herein are made to the FGF1 portion of the FGF2/FGF1 chimera. Although mutations below are noted by a particular amino acid for example in SEQ ID NO: 6, 8 or 14, one skilled in the art will appreciate that the corresponding amino acid can be mutated in any FGF1 sequence. For example, Q40 of SEQ ID NO: 14 corresponds to Q55 of SEQ ID NO: 6 and 8.

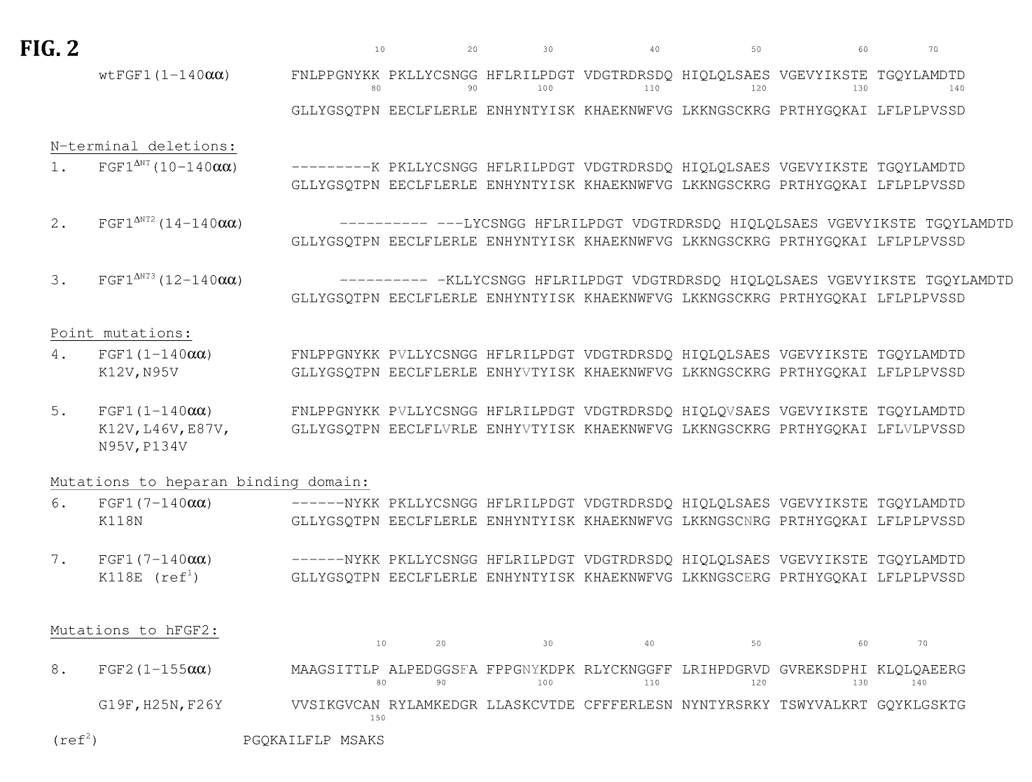

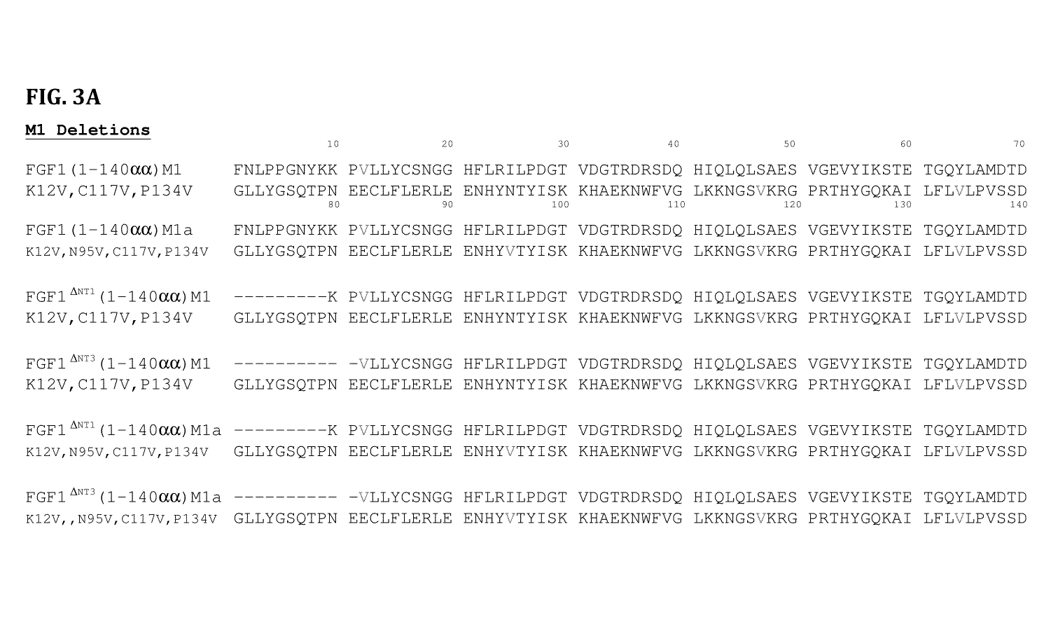

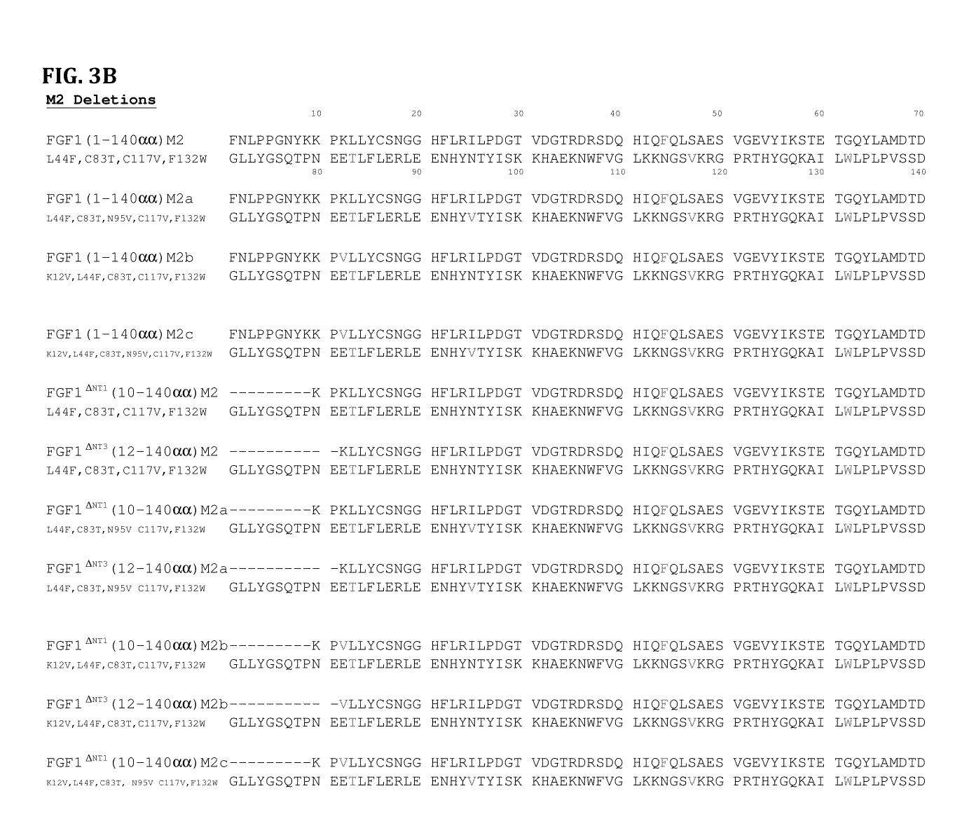

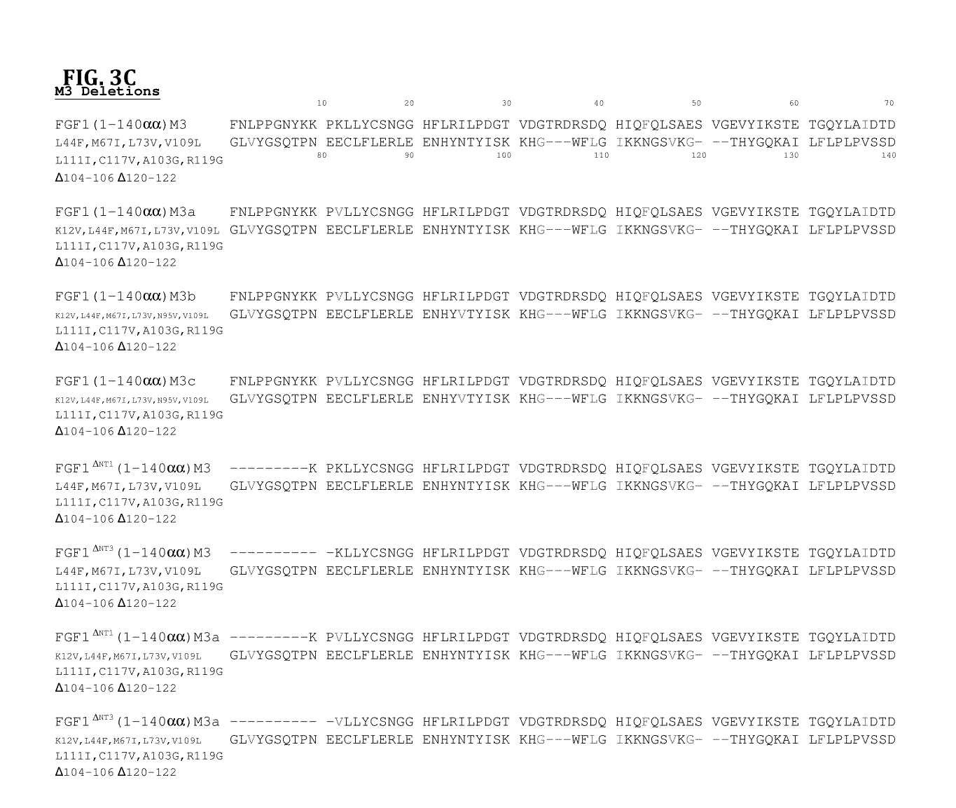

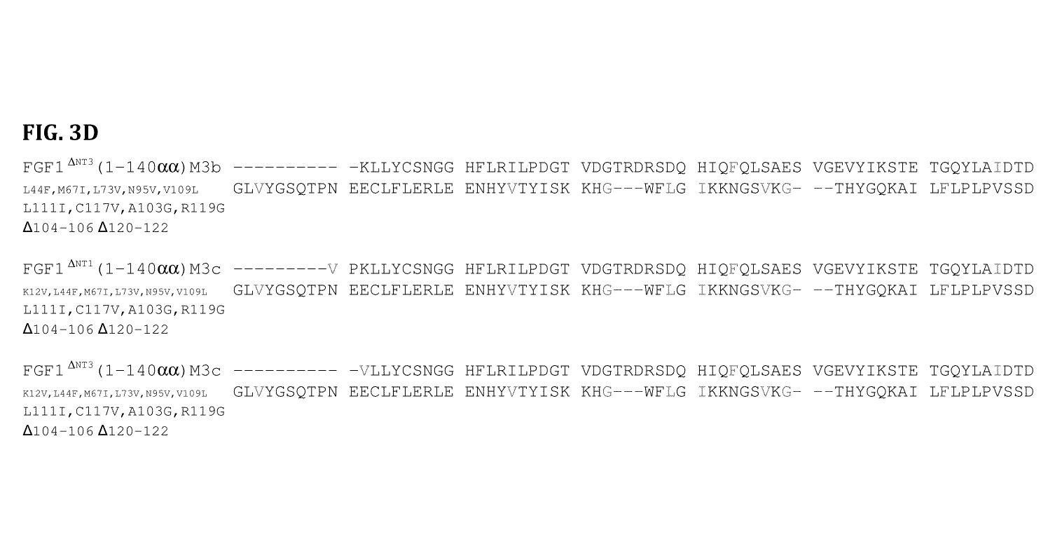

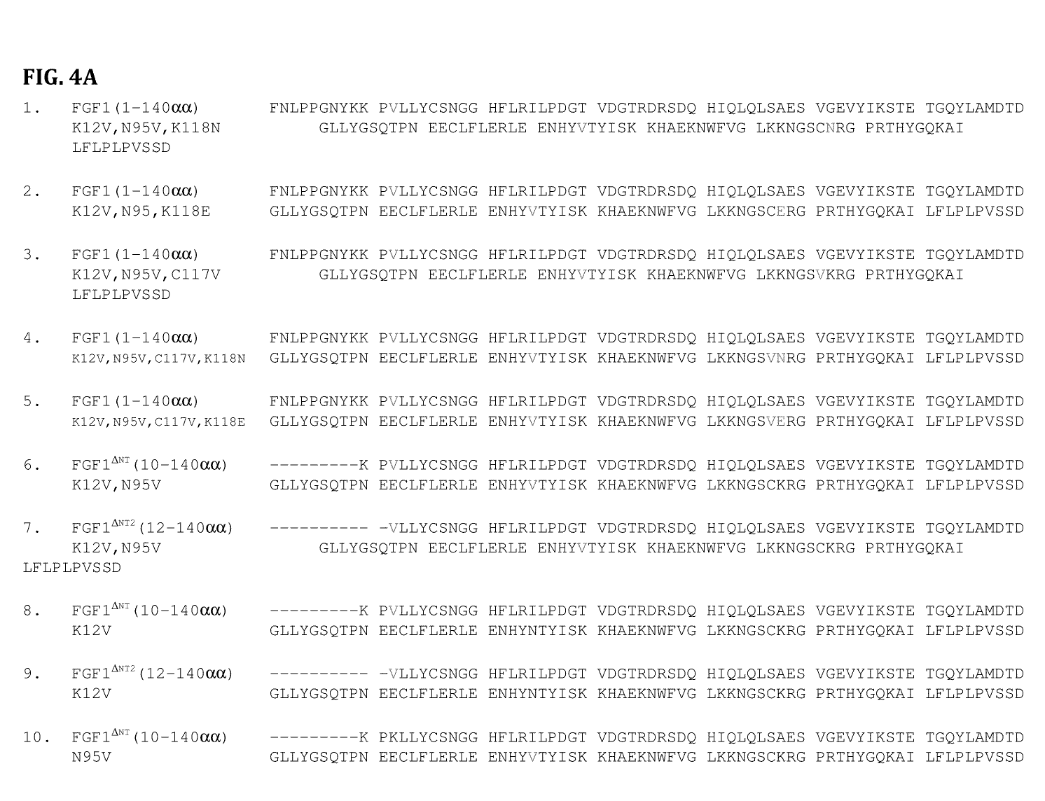

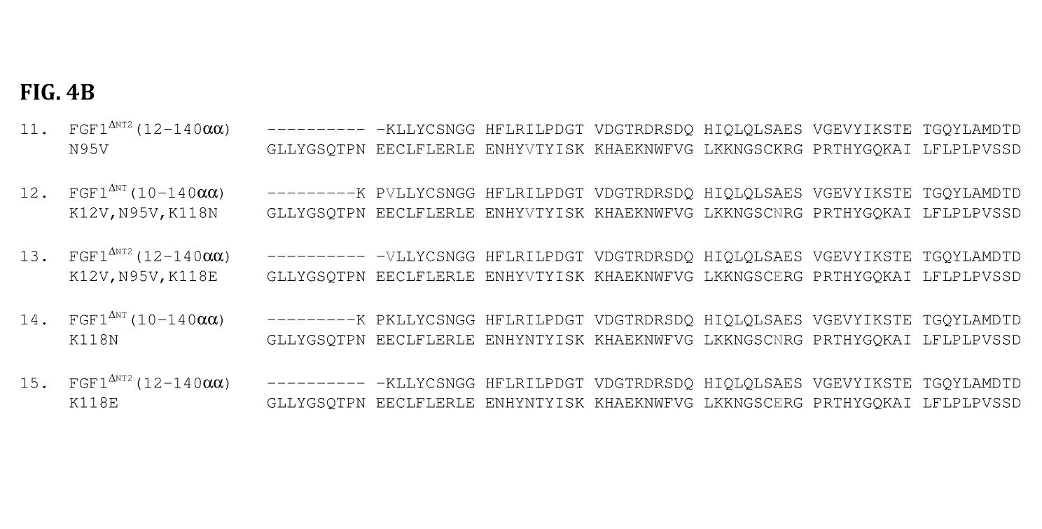

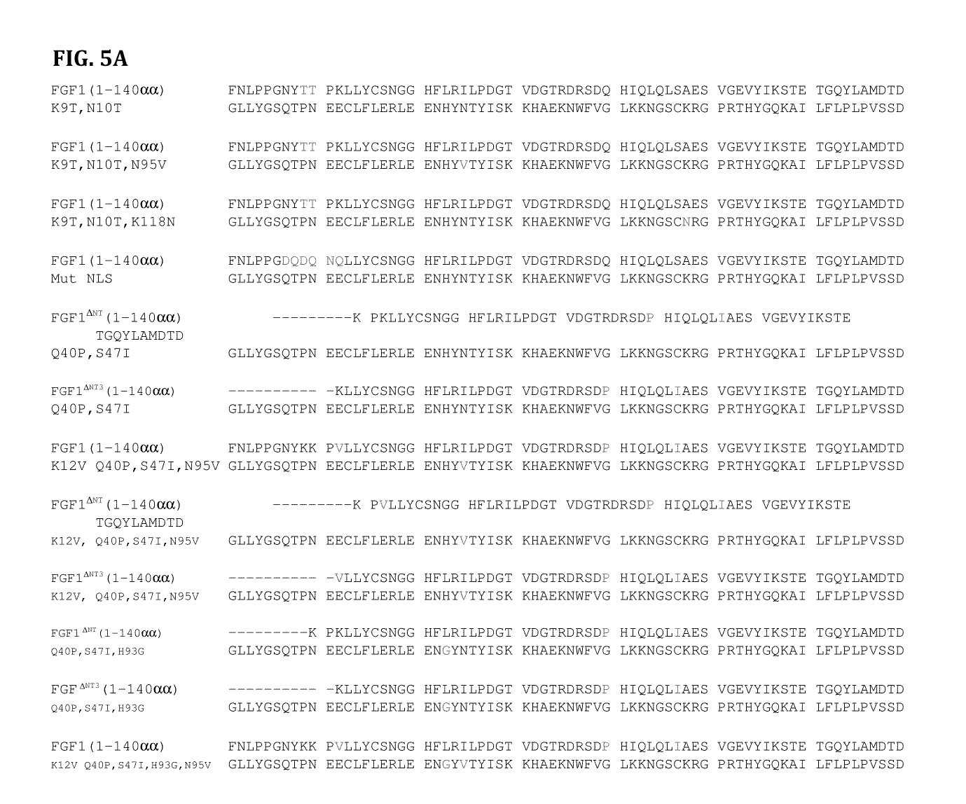

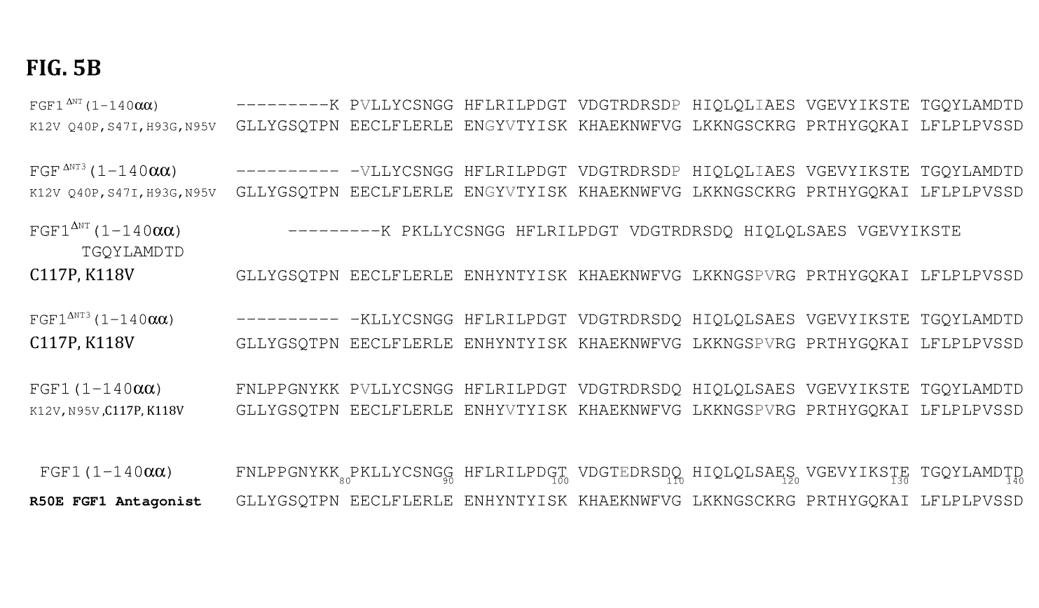

In one example, mutations are made to the N-terminal region of FGF1 (such as SEQ ID NO: 6, 8 or 14), such as deletion of the first 14, 15, 16, 17, 18, 19, 20, 21, 22, 23, 24, 25, 26, 27, 28, 29, or 30 amino acids of SEQ ID NO: 6 or 8 (such as deletion of at least the first 14 amino acids of SEQ ID NO: 6 or 8, such as deletion of at least the first 15, at least 16, at least 20, at least 25, or at least 29 amino acids of SEQ ID NO: 6 or 8), deletion of the first 3, 4, 5, 6, 7, 8, 9, 10, 11, 12, 13, 14, 15, 16, 17, 18, 19, or 20 amino acids of SEQ ID NO: 14 (e.g., see SEQ ID NOS: 15-17 and FIG. 2).

In some examples, the FGF1 portion of the FGF2/FGF1 chimera includes mutations at one or more of the following positions of FGF1: K9, K10, K12, L14, Y15, C16, H21, R35, Q40, L44, L46, S47, E49, Y55, M67, L73, C83, L86, E87, H93, Y94, N95, H102, A103, E104, K105, N106, F108, V109, L111, K112, K113, C117, K118, R119, G120, P121, R122, F132, L133, P134, L135, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or all 42 of these positions. In some examples, the FGF1 portion of the FGF2/FGF1 chimera includes mutations at one or more of the following positions of FGF1: K12, R35, Y55, E87, Y94, N95, and C117 such as 1, 2, 3, 4, 5, 6, or 7 of these positions. In some examples, the FGF1 portion of the FGF2/FGF1 chimera includes mutations at one or more of the following positions of FGF1: K112, K113, K118 and such as 1, 2, or 3 of these positions.

Mutations can be made to FGF1 (such as SEQ ID NO: 6, 8 or 14) to reduce its mitogenic activity. In some examples, such mutations reduce mitogenic activity by at least 50%, at least 60%, at least 70%, at least 75%, at least 80%, at least 90%, at least 92%, at least 95%, at least 98%, at least 99%, or even complete elimination of detectable mitogenic activity. Methods of measuring mitogenic activity are known in the art, such as thymidine incorporation into DNA in serum-starved cells (e.g., NIH 3T3 cells) stimulated with the mutated FGF1, methylthiazoletetrazolium (MTT) assay, cell number quantification or BrdU incorporation. In some examples, the assay provided by Fu et al., World J. Gastroenterol. 10:3590-6, 2004; Klingenberg et al., J. Biol. Chem. 274:18081-6, 1999; Shen et al., Protein Expr Purif. 81:119-25, 2011, or Zou et al., Chin. Med. J. 121:424-429, 2008 is used to measure mitogenic activity. Examples of such mutations include, but are not limited to K12C, K12V, L46V, E87V, N95V, K12V/N95V (e.g., see SEQ ID NO: 18, which can also include a methionine on its N-terminus), and Lys12Val/Pro134Val, Lys12Val/Leu46Val/Glu87Val/Asn95Val/Pro134Val (e.g., see SEQ ID NO: 19, which can also include a methionine on its N-terminus) (wherein the numbering refers to the sequence shown SEQ ID NO: 14). In some examples, a portion of contiguous N-terminal residues are removed, such as amino acids 1-9 of SEQ ID NO: 14, to produce a non-mitogenic form of FGF21. An example is shown in SEQ ID NO: 27. Mutations that reduce the heparan binding affinity (such as a reduction of at least 10%, at least 20%, at least 50%, or at least 75%), can also be used to reduce mitogenic activity, for example by substituting heparan binding residues from a paracrine FGFs into FGF1.

Mutations can also be introduced into one or both nuclear localization sites (NLS1, amino acids 24-27 of SEQ ID NO: 6 and NLS2, amino acids 115-128 of SEQ ID NO: 6) of FGF1, for example to reduce mitogenicity. Examples of NLS mutations that can be made to FGF1 include but are not limited to: deleting or mutating all or a part of NLS1 (such as deleting or mutating the lysines), deleting or mutating the lysines in NLS2 such as ¹¹⁵KK . . . ¹²⁷KK . . . , or combinations thereof (wherein the numbering refers to the sequence shown SEQ ID NO: 6). For example, one or more of 24K, 25K, 27K, 115K, 116K, 127K or 128K (wherein the numbering refers to the sequence shown SEQ ID NO: 6) or can be mutated (for example changed to an alanine or deleted). Particular examples of such mutations that can be made to the heparan binding site in the NLS2 (KKN . . . KR) domain are shown in SEQ ID NOS: 20 and 21 (K118N or K118E, respectively, wherein numbering refers to SEQ ID NO: 14).

Mutations can be introduced into the phosphorylation site of FGF1, for example to create a constitutively active or inactive mutant to affects nuclear signaling.

In some examples, mutations are introduced into the FGF1 nuclear export sequence, for example to increase the amount of FGF1 in the nucleus and reduce its mitogenicity as measured by thymidine incorporation assays in cultured cells (e.g., see Nilsen et al., J. Biol. Chem. 282(36):26245-56, 2007). Mutations to the nuclear export sequence decrease FGF1-induced proliferation (e.g., see Nilsen et al., J. Biol. Chem. 282(36):26245-56, 2007). Methods of measuring FGF1 degradation are known in the art, such as measuring [³⁵S]Methionine-labeled FGF1 or immunoblotting for steady-state levels of FGF1 in the presence or absence of proteasome inhibitors. In one example, the assay provided by Nilsen et al., J. Biol. Chem. 282(36):26245-56, 2007 or Zakrzewska et al., J. Biol. Chem. 284:25388-403, 2009 is used to measure FGF1 degradation.

The FGF1 nuclear export sequence includes amino acids 145-152 of SEQ ID NO: 6 and 8 or amino acids 130-137 of SEQ ID NO: 14. Examples of FGF1 nuclear export sequence mutations that can be made to include but are not limited to changing the sequence ILFLPLPV (amino acids 145-152 of SEQ ID NO: 6 and 8) to AAALPLPV (SEQ ID NO: 23), ILALPLPV (SEQ ID NO: 24), ILFAPLPV (SEQ ID NO: 25), or ILFLPAPA (SEQ ID NO: 26).

In one example, mutations are introduced to improve stability of FGF1. In some examples, the sequence NYKKPKL (amino acids 22-28 of SEQ ID NO: 6) is not altered, and in some examples ensures for structural integrity of FGF1 and increases interaction with the FGF1 receptor. Methods of measuring FGF1 stability are known in the art, such as measuring denaturation of FGF1 or mutants by fluorescence and circular dichroism in the absence and presence of a 5-fold molar excess of heparin in the presence of 1.5 M urea or isothermal equilibrium denaturation by guanidine hydrochloride. In one example, the assay provided by Dubey et al., J. Mol. Biol. 371:256-268, 2007 is used to measure stability of the protein. Examples of mutations that can be introduced into FGF1 to increase stability include, but are not limited to, one or more of Q40P, S47I and H93G (wherein the numbering refers to the sequence shown SEQ ID NO: 14).

In some examples, mutations in FGF1 increase the thermostability of mature or truncated FGF1. For example, mutations can be made at one or more of the following positions. Exemplary mutations that can be used to increase the thermostability of mutated FGF1 include but are not limited to one or more of: K12, C117, P134, L44, C83, F132, M67, L73, V109, L111, A103, R119, 4104-106, and Δ120-122, wherein the numbering refers to SEQ ID NO: 14 (e.g., see Xia et al., PLoS One. 7:e48210, 2012). In some examples, thermostability of FGF1 is increased by using one or more of the following mutations: Q40P and S47I or Q40P, S47I, and H93G (or any other combination of these mutations).

In one example, mutations are introduced to increase protease resistance of FGF1 (e.g., see Kobielak et al., Protein Pept Lett. 21(5):434-43, 2014). Other mutations that can be made to FGF include those mutations provided in Lin et al., J Biol Chem. 271(10):5305-8, 1996).

In some examples, the mutant FGF1 protein or chimera is PEGylated at one or more positions, such as at N95 (for example see methods of Niu et al., J. Chromatog. 1327:66-72, 2014, herein incorporated by reference). Pegylation consists of covalently linking a polyethylene glycol group to surface residues and/or the N-terminal amino group. N95 is known to be involved in receptor binding, thus is on the surface of the folded protein. As mutations to surface exposed residues could potentially generate immunogenic sequences, pegylation is an alternative method to abrogate a specific interaction. Pegylation is an option for any surface exposed site implicated in the receptor binding and/or proteolytic degradation. Pegylation can “cover” functional amino acids, e.g. N95, as well as increase serum stability.

In some examples, the mutant FGF1 protein or chimera includes an immunoglobin FC domain (for example see Czajkowsky et al., EMBO Mol. Med. 4:1015-28, 2012, herein incorporated by reference). The conserved FC fragment of an antibody can be incorporated either n-terminal or c-terminal of the mutant FGF1 protein or chimera, and can enhance stability of the protein and therefore serum half-life. The FC domain can also be used as a means to purify the proteins on protein A or Protein G sepharose beads. This makes the FGF1 mutants having heparin binding mutations easier to purify.

### Variant Sequences

Variant FGF2/FGF1 chimeric proteins, including variants of the FGF2 and FGF1 fragments in Table 1, and variants of SEQ ID NOS: 9, 10, 11, 12, 13, 99, 100, 101, 102, 103, and 104, as well as variants of FGF2/FGF1 chimers that further include a 3-Klotho-binding peptide and/or FGFR1c-binding peptide (such as any of SEQ ID NOS: 156, 157, 158, 159, 160, 161, 162, 163, 164, 165, 166 or 167), can contain one or more mutations, such as a single insertion, a single deletion, a single substitution, or combinations thereof. In some examples, the FGF2 fragment includes 1-4 deletions, 1-4 insertions, 1-4 substitutions, or any combination thereof (e.g., single deletion together with 1-3 insertions), however in some examples such variants retain the ability to lower blood glucose in a mammal. In some examples, the disclosure provides a variant of any disclosed FGF2 fragment having 1, 2, 3, or 4 amino acid changes. In some examples, the FGF1 fragment includes 1-20 insertions, 1-20 deletions, 1-20 substitutions, or any combination thereof (e.g., single insertion together with 1-19 substitutions). In some examples, the disclosure provides a variant of any disclosed FGF1 fragment having 1, 2, 3, 4, 5, 6, 7, 8, 9, 10, 11, 12, 13, 14, 15, 16, 17, 18, 19 or 20 amino acid changes. In some examples, any of SEQ ID NOS: 9, 10, 11, 12, 13, 99, 100, 101, 102, 103, 104, 156, 157, 158, 159, 160, 161, 162, 163, 164, 165, 166 or 167 can include 1 to 20 or 1 to 10 insertions, 1 to 20 or 1 to 10 deletions, 1 to 20 or 1 to 10 substitutions, or any combination thereof (e.g., single insertion together with 1 to 7 substitutions). In some examples, the disclosure provides a variant of SEQ ID NO: 9, 10, 11, 12, 13, 99, 100, 101, 102, 103, or 104 having 1, 2, 3, 4, 5, 6, 7, 8, 9, 10, 11, 12, 13, 14, 15, 16, 17, 18, 19 or 20 amino acid changes. In some examples, the disclosure provides a variant of any disclosed β-Klotho-binding peptides having 1-20 insertions, 1-20 deletions, 1-20 substitutions, or any combination thereof (e.g., single insertion together with 1-19 substitutions). In some examples, the disclosure provides a variant of any disclosed β-Klotho-binding peptides having 1, 2, 3, 4, 5, 6, 7, 8, 9, 10, 11, 12, 13, 14, 15, 16, 17, 18, 19 or 20 amino acid changes (e.g., in SEQ ID NO: 105, 106, 107, 108, 109, 110, 111, 112, 113, 114, 115, 116, 117, 118, 119, 120, 121, 122, 123, 124, 125, 126, 127, 128, 129, or 130). In some examples, the disclosure provides a variant of any disclosed FGFR1c-binding peptides having 1-20 insertions, 1-20 deletions, 1-20 substitutions, or any combination thereof (e.g., single insertion together with 1-19 substitutions). In some examples, the disclosure provides a variant of any disclosed FGFR1c-binding peptides having 1, 2, 3, 4, 5, 6, 7, 8, 9, 10, 11, 12, 13, 14, 15, 16, 17, 18, 19 or 20 amino acid changes (e.g., in SEQ ID NO: 131, 132, 133, 134, 135, 136, 137, 138, 139, 140, 141, 142, 143, 144, 145, 146, 147, 148, 149, 150 or 151). In some examples, the disclosure provides a variant of any disclosed β-Klotho-binding/FGFR1c-binding chimera having 1-20 insertions, 1-20 deletions, 1-20 substitutions, or any combination thereof (e.g., single insertion together with 1-19 substitutions). In some examples, the disclosure provides a variant of any disclosed β-Klotho-binding/FGFR1c-binding chimera having 1, 2, 3, 4, 5, 6, 7, 8, 9, 10, 11, 12, 13, 14, 15, 16, 17, 18, 19 or 20 amino acid changes (e.g., in SEQ ID NO: 152, 153, 154, or 155). In one example, such variant peptides are produced by manipulating the nucleotide sequence encoding a peptide using standard procedures such as site-directed mutagenesis or PCR. Such variants can also be chemically synthesized.

One type of modification or mutation includes the substitution of amino acids for amino acid residues having a similar biochemical property, that is, a conservative substitution (such as 1 to 4, 1 to 5, 1 to 8, 1 to 10, or 1 to 20 conservative substitutions). Typically, conservative substitutions have little to no impact on the activity of a resulting peptide. For example, a conservative substitution is an amino acid substitution in SEQ ID NO: SEQ ID NO: 9, 10, 11, 12, 13, 99, 100, 101, 102, 103, 104, 156, 157, 158, 159, 160, 161, 162, 163, 164, 165, 166 or 167 that does not substantially affect the ability of the peptide to decrease blood glucose in a mammal. An alanine scan can be used to identify which amino acid residues in an FGF2 fragment, FGF1 fragment, β-Klotho-binding protein, or FGFR1c-binding protein or any of SEQ ID NOS: SEQ ID NO: 9, 10, 11, 12, 13, 99, 100, 101, 102, 103, 104, 105, 106, 107, 108, 109, 110, 111, 112, 113, 114, 115, 116, 117, 118, 119, 120, 121, 122, 123, 1124, 125, 126, 127, 128 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166 or 167 can tolerate an amino acid substitution. In one example, the activity of FGF2, FGF1, β-Klotho-binding protein, or FGFR1c-binding protein, or any of SEQ ID NOS: 9-13 and 99-167 is not altered by more than 25%, for example not more than 20%, for example not more than 10%, when an alanine, or other conservative amino acid, is substituted for 1-4, 2-3, 1-5, 1-8, 1-10, or 1-20 native amino acids. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions or additions) can be assessed for fragments of FGF1 or FGF2 by analyzing the function of the protein, as well as any chimera made using the sequences in Table 1, 2 and/or 3, or SEQ ID NOS: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175 (or variants thereof), such as SEQ ID NOS: 9, 10, 11, 12, 13, 99, 100, 101, 102, 103, and 104, by analyzing the ability of the variant protein to decrease blood glucose in a mammal.

### Spacers

In some embodiments, the FGF2/FGF1 chimeric protein includes an FGF2 portion contiguously joined to an FGF1 portion. However, one skilled in the art will appreciate that in some examples, the FGF2 portion and the FGF1 portion are coupled by an intervening spacer, such as a peptide sequence of 1, 2, 3, 4, 5, 6, 7, 8, 9, 10, or more amino acid residues. Thus, a spacer could be introduced between the FGF2 and FGF1 sequences shown in Table 1. Similarly, SEQ ID NO: 9, 10, 11, 12, 13, 99, 100, 101, 102, 103, 104, 156, 157, 158, 159, 160, 161, 162, 163, 164, 165, 166 or 167 can be modified to include such a spacer between the FGF2 and FGF1 portion, between a (3-Klotho-binding peptide and FGF2 and/or FGF1, between a FGFR1c-binding peptide and FGF2 and/or FGF1, and/or between a FGFR1c-binding peptide and a β-Klotho-binding peptide. For example, SEQ ID NO: 9 can be modified by introducing a spacer ([spacer]) as follows:

MAAGSITTL PALPEDGGSG AF [spacer] PPGNYK KPKLLYCSNG
GHFLRILPDG TVDGTRDRSD QHIQLQLSAE SVGEVYIKST
ETGQYLAMDT DGLLYGSQTP NEECLFLERL EENHYNTYIS
KKHAEKNWFVGLKKNGSCKR GPRTHYGQKA ILFLPLPVSSD

### Addition of Other Peptides

In some examples, the FGF2/FGF1 chimeric protein includes other proteins or peptides, for example at its N-terminus, at its C-terminus or both at its N- and its C-terminus. For example, any FGF2/FGF1 chimeric protein provided herein can be joined directly or indirectly to the end of a β-Klotho-binding protein, an FGFR1c binding protein, or both a 3-Klotho-binding protein, an FGFR1c binding protein. Examples of β-Klotho-binding proteins that can be directly or indirectly attached to a FGF2/FGF1 chimeric protein are shown in SEQ ID NO: 105, 106, 107, 108, 109, 110, 111, 112, 113, 114, 115, 116, 117, 118, 119, 120, 121, 122, 123, 124, 125, 126, 127, 128, 129, and 130. Examples of FGFR1c-binding proteins that can be directly or indirectly attached to a FGF2/FGF1 chimeric protein are shown in SEQ ID NOS: 131, 132, 133, 134, 135, 136, 137, 138, 139, 140, 141, 142, 143, 144, 145, 146, 147, 148, 149, 150 and 151. Examples of β-Klotho-binding/FGFR1c-binding protein chimeras that can be directly or indirectly attached to a FGF2/FGF1 chimeric protein are shown in SEQ ID NOS: 152, 153, 154, and 155. Specific examples of FGF2/FGF1 chimeric proteins with other peptides are shown in SEQ ID NOS: 156, 157, 158, 159, 160, 161, 162, 163, 164, 165, 166 and 167. However, based on this disclosure, such as FIGS. 19-22, one skilled in the art will recognize that any FGF2/FGF1 chimera provided herein can be directly or indirectly linked to any β-Klotho-binding protein and/or any FGFR1c-binding protein provided herein.

The FGF2/FGF1 chimeric protein can be contiguously joined to a β-Klotho-binding protein portion. However, one skilled in the art will appreciate that in some examples, the FGF2/FGF1 portion and the β-Klotho-binding protein portion are coupled by an intervening spacer, such as a peptide sequence of 1, 2, 3, 4, 5, 6, 7, 8, 9, 10, or more amino acid residues. Thus, a spacer could be introduced between the FGF2/FGF1 and β-Klotho-binding protein. Similarly, the FGF2/FGF1 chimeric protein can be contiguously joined to an FGFR1c-binding protein portion. However, one skilled in the art will appreciate that in some examples, the FGF2/FGF1 portion and the FGFR1c-binding protein portion are coupled by an intervening spacer, such as a peptide sequence of 1, 2, 3, 4, 5, 6, 7, 8, 9, 10, or more amino acid residues. Similarly, the FGF2/FGF1 chimeric protein can be contiguously joined to both an FGFR1c-binding protein portion and a β-Klotho-binding protein portion. However, one skilled in the art will appreciate that in some examples, the FGF2/FGF1 portion, the β-Klotho-binding protein portion, and/or the FGFR1c-binding protein portion are coupled by an intervening spacer, such as a peptide sequence of 1, 2, 3, 4, 5, 6, 7, 8, 9, 10, or more amino acid residues. Thus, a spacer could be introduced between the FGF2/FGF1 portion, the β-Klotho-binding protein portion, and/or the FGFR1c-binding protein portion. Similarly, any of SEQ ID NOS: 156, 157, 158, 159, 160, 161, 162, 163, 164, 165, 166 or 167 can be modified to include such a spacer(s).

### Production of Proteins

Isolation and purification of recombinantly expressed FGF2/FGF1 chimeric proteins can be carried out by conventional means including preparative chromatography and immunological separations. Once expressed, FGF2/FGF1 chimeric proteins can be purified according to standard procedures of the art, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes.

In addition to recombinant methods, FGF2/FGF1 chimeric proteins disclosed herein can also be constructed in whole or in part using standard peptide synthesis. In one example, FGF2/FGF1 chimeric proteins are synthesized by condensation of the amino and carboxyl termini of shorter fragments. Methods of forming peptide bonds by activation of a carboxyl terminal end (such as by the use of the coupling reagent N, N′-dicylohexylcarbodimide) are well known in the art.

### FGF2/FGF1 Chimeric Nucleic Acid Molecules and Vectors

Nucleic acid molecules encoding an FGF2/FGF1 chimeric protein, including those that further include a β-Klotho-binding peptide and/or FGFR1c-binding peptide, are encompassed by this disclosure. Based on the genetic code, nucleic acid sequences coding for any FGF2/FGF1 chimeric sequence, such as those generated using the sequences shown in Table 1, 2 or 3, as well as the sequences in SEQ ID NOS: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, can be routinely generated. Similarly, nucleic acid sequences coding for any FGF2/FGF1 chimeric proteins that further include a β-Klotho-binding peptide and/or FGFR1c-binding peptide such as those generated using the sequences shown in SEQ ID NOS: 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166 or 167, can be routinely generated. In some examples, such a sequence is optimized for expression in a host cell, such as a host cell used to express the chimeric protein.

In one example, a nucleic acid sequence coding for an FGF2/FGF1 chimeric protein has at least 60%, at least 70%, at least 75%, at least 80%, at least 90%, at least 92%, at least 95%, at least 96%, at least 97%, at least 99% or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167 can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same chimeric protein sequence. In one example, a chimeric FGF2/FGF1 nucleic acid sequence has at least 70%, at least 80%, at least 85%, at least 90%, at least 92%, at least 95%, at least 98%, or at least 99% sequence identity to SEQ ID NO: 31, 32, or 33.

Nucleic acid molecules include DNA, cDNA and RNA sequences which encode a FGF2/FGF1 chimeric peptide, including those that further include a β-Klotho-binding peptide and/or FGFR1c-binding peptide. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3^{rd} Edition, W.H. 5 Freeman and Co., NY).

Codon preferences and codon usage tables for a particular species can be used to engineer isolated nucleic acid molecules encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) that take advantage of the codon usage preferences of that particular species. For example, the chimeric proteins disclosed herein can be designed to have codons that are preferentially used by a particular organism of interest.

A nucleic acid encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a nucleic acid molecule encoding a portion of an FGF2/FGF1 chimeric protein can be isolated by polymerase chain reaction of cDNA using primers based on the DNA sequence of FGF1 and FGF2, and the desired sequences ligated together to form the chimera. A wide variety of cloning and in vitro amplification methodologies are well known to persons skilled in the art. In addition, nucleic acids encoding sequences encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175 or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be prepared by cloning techniques. Examples of appropriate cloning and sequencing techniques, and instructions sufficient to direct persons of skill through cloning are found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.

Nucleic acid sequences encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

In one example, an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) is prepared by inserting the cDNA which encodes the chimeric protein into a vector. The insertion can be made so that the individual portion of the chimeric protein (such as the N- and C-terminal portions) are read in frame so that one continuous FGF2/FGF1 chimeric protein is produced.

The FGF2/FGF1 chimeric protein nucleic acid coding sequence, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect, plant and mammalian organisms. Methods of expressing DNA sequences having eukaryotic or viral sequences in prokaryotes are well known in the art. Biologically functional viral and plasmid DNA vectors capable of expression and replication in a host are known in the art. The vector can encode a selectable marker, such as a thymidine kinase gene.

Nucleic acid sequences encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be operatively linked to expression control sequences. An expression control sequence operatively linked to an FGF2/FGF1 chimeric protein coding sequence is ligated such that expression of the chimeric protein coding sequence is achieved under conditions compatible with the expression control sequences. The expression control sequences include, but are not limited to appropriate promoters, enhancers, transcription terminators, a start codon (i.e., ATG) in front of an FGF2/FGF1 chimeric protein-encoding gene, splicing signal for introns, maintenance of the correct reading frame of that gene to permit proper translation of mRNA, and stop codons.

In one embodiment, vectors are used for expression in yeast such as S. cerevisiae, P. pastoris, or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2p) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The nucleic acid molecules encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can also be designed to express in insect cells.

An FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast cell lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared that encode an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167). Exemplary viral vectors include polyoma, SV40, adenovirus, vaccinia virus, adeno-associated virus, herpes viruses including HSV and EBV, Sindbis viruses, alphaviruses and retroviruses of avian, murine, and human origin. Baculovirus (Autographa californica multinuclear polyhedrosis virus; AcMNPV) vectors are also known in the art, and may be obtained from commercial sources. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

Viral vectors that encode an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can include at least one expression control element operationally linked to the nucleic acid sequence encoding the FGF2/FGF1 chimeric protein. The expression control elements are inserted in the vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the FGF2/FGF1 chimeric protein in the host system. The expression vector can contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Basic techniques for preparing recombinant DNA viruses containing a heterologous DNA sequence encoding the FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) are known. Such techniques involve, for example, homologous recombination between the viral DNA sequences flanking the DNA sequence in a donor plasmid and homologous sequences present in the parental virus. The vector can be constructed for example by steps known in the art, such as by using a unique restriction endonuclease site that is naturally present or artificially inserted in the parental viral vector to insert the heterologous DNA.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing FGF2/FGF1 chimeric proteins, including those that further include a β-Klotho-binding peptide and/or FGFR1c-binding peptide, in cells including higher eukaryotic cells such as the COS, CHO, HeLa and myeloma cell lines.

### Cells Expressing FGF2/FGF1 Chimeric Proteins

A nucleic acid molecule encoding an FGF2/FGF1 chimeric protein disclosed herein, including those that further include a β-Klotho-binding peptide and/or FGFR1c-binding peptide, can be used to transform cells and make transformed cells. Thus, cells expressing an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) are disclosed. Cells expressing an FGF2/FGF1 chimeric protein disclosed herein, including those that further include a β-Klotho-binding peptide and/or FGFR1c-binding peptide, can be eukaryotic or prokaryotic. Examples of such cells include, but are not limited to bacteria, archea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, SF9 cells, C129 cells, 293 cells, Neurospora, and immortalized mammalian myeloid and lymphoid cell lines.

Cells expressing an FGF2/FGF1 chimeric protein are transformed or recombinant cells. Such cells can include at least one exogenous nucleic acid molecule that encodes an FGF2/FGF1 chimeric protein, including those that further include a β-Klotho-binding peptide and/or FGFR1c-binding peptide, for example a sequence encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167. It is understood that all progeny may not be identical to the parental cell since there may be mutations that occur during replication. Methods of stable transfer, meaning that the foreign DNA is continuously maintained in the host cell, are known in the art.

Transformation of a host cell with recombinant DNA may be carried out by conventional techniques as are well known. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. Techniques for the transformation of yeast cells, such as polyethylene glycol transformation, protoplast transformation and gene guns are also known in the art.

### Pharmaceutical Compositions that Include FGF2/FGF1 Chimeras

Pharmaceutical compositions that include an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) or a nucleic acid encoding these proteins, can be formulated with an appropriate pharmaceutically acceptable carrier, depending upon the particular mode of administration chosen.

In some embodiments, the pharmaceutical composition consists essentially of an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) (or a nucleic acid encoding such a protein) and a pharmaceutically acceptable carrier. In these embodiments, additional therapeutically effective agents are not included in the compositions.

In other embodiments, the pharmaceutical composition includes an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) (or a nucleic acid encoding such a protein) and a pharmaceutically acceptable carrier. Additional therapeutic agents, such as agents for the treatment of diabetes, can be included. Thus, the pharmaceutical compositions can include a therapeutically effective amount of another agent. Examples of such agents include, without limitation, anti-apoptotic substances such as the Nemo-Binding Domain and compounds that induce proliferation such as cyclin dependent kinase (CDK)-6, CDK-4 and cyclin Dl. Other active agents can be utilized, such as antidiabetic agents for example, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570) and antagonists, PPAR-gamma/alpha modulators (such as KRP 297), alpha-glucosidase inhibitors (e.g., acarbose, voglibose), dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g. exendin-4) or amylin. Additional examples include immunomodulatory factors such as anti-CD3 mAb, growth factors such as HGF, VEGF, PDGF, lactogens, and PTHrP. In some examples, the pharmaceutical compositions containing an FGF2/FGF1 chimeric protein, including those that further include a β-Klotho-binding peptide and/or FGFR1c-binding peptide, can further include a therapeutically effective amount of other FGFs, such as FGF21, FGF19, or both, heparin, or combinations thereof.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, an FGF2/FGF1 chimeric protein, including those that further include a 3-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) is included in a controlled release formulation, for example, a microencapsulated formulation. Various types of biodegradable and biocompatible polymers, methods can be used, and methods of encapsulating a variety of synthetic compounds, proteins and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other embodiments, an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) is included in a nanodispersion system. Nanodispersion systems and methods for producing such nanodispersions are well known to one of skill in the art. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006. With regard to the administration of nucleic acids, one approach to administration of nucleic acids is direct treatment with plasmid DNA, such as with a mammalian expression plasmid. As described above, the nucleotide sequence encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be placed under the control of a promoter to increase expression of the protein.

Many types of release delivery systems are available and known. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or polynucleotide encoding this chimeric protein, is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant may be particularly suitable for treatment of chronic conditions, such as diabetes. Long-term release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. These systems have been described for use with nucleic acids (see U.S. Pat. No. 6,218,371). For use in vivo, nucleic acids and peptides are preferably relatively resistant to degradation (such as via endo- and exo-nucleases). Thus, modifications of the disclosed FGF2/FGF1 chimeric proteins, such as the inclusion of a C-terminal amide, can be used.

The dosage form of the pharmaceutical composition can be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The pharmaceutical compositions that include an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be formulated in unit dosage form, suitable for individual administration of precise dosages. In one non-limiting example, a unit dosage contains from about 1 mg to about 1 g of an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), such as about 10 mg to about 100 mg, about 50 mg to about 500 mg, about 100 mg to about 900 mg, about 250 mg to about 750 mg, or about 400 mg to about 600 mg. In other examples, a therapeutically effective amount of an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) is about 0.01 mg/kg to about 50 mg/kg, for example, about 0.5 mg/kg to about 25 mg/kg or about 1 mg/kg to about 10 mg/kg. In other examples, a therapeutically effective amount of an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) is about 1 mg/kg to about 5 mg/kg, for example about 2 mg/kg. In a particular example, a therapeutically effective amount of an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) includes about 1 mg/kg to about 10 mg/kg, such as about 2 mg/kg.

### Treatment Using FGF2/FGF1 Chimeras

The disclosed FGF2/FGF1 chimeric proteins, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or nucleic acids encoding such proteins, can be administered to a subject, for example to treat a metabolic disease, for example by reducing fed and fasting blood glucose, improving insulin sensitivity and glucose tolerance, reducing systemic chronic inflammation, ameliorating hepatic steatosis in a mammal, or combinations thereof.

The compositions of this disclosure that include an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) (or nucleic acids encoding these molecules) can be administered to humans or other animals by any means, including orally, intravenously, intramuscularly, intraperitoneally, intranasally, intradermally, intrathecally, subcutaneously, via inhalation or via suppository. In one non-limiting example, the composition is administered via injection. In some examples, site-specific administration of the composition can be used, for example by administering an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) (or a nucleic acid encoding these molecules) to pancreas tissue (for example by using a pump, or by implantation of a slow release form at the site of the pancreas). The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly or monthly etc.) doses over a period of a few days to months, or even years. For example, a therapeutically effective amount of an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be administered in a single dose, twice daily, weekly, or in several doses, for example daily, or during a course of treatment. In a particular non-limiting example, treatment involves once daily dose or twice daily dose.

The amount of FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) administered can be dependent on the subject being treated, the severity of the affliction, and the manner of administration, and is best left to the judgment of the prescribing clinician. Within these bounds, the formulation to be administered will contain a quantity of the FGF2/FGF1 chimeric protein in amounts effective to achieve the desired effect in the subject being treated. A therapeutically effective amount of FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be the amount of the chimeric protein, or a nucleic acid encoding these molecules that is necessary to treat diabetes or reduce blood glucose levels (for example a reduction of at least 20%).

When a viral vector is utilized for administration of an nucleic acid encoding an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), the recipient can receive a dosage of each recombinant virus in the composition in the range of from about 10⁵ to about 10¹⁰ plaque forming units/mg mammal, although a lower or higher dose can be administered. Examples of methods for administering the composition into mammals include, but are not limited to, exposure of cells to the recombinant virus ex vivo, or injection of the composition into the affected tissue or intravenous, subcutaneous, intradermal or intramuscular administration of the virus. Alternatively the recombinant viral vector or combination of recombinant viral vectors may be administered locally by direct injection into the pancreases in a pharmaceutically acceptable carrier. Generally, the quantity of recombinant viral vector, carrying the nucleic acid sequence of the FGF2/FGF1 chimeric protein to be administered, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) is based on the titer of virus particles. An exemplary range to be administered is 10⁵ to 10¹⁰ virus particles per mammal, such as a human.

In some examples, the FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175 or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or a nucleic acid encoding the FGF2/FGF1 chimeric protein, is administered in combination (such as sequentially or simultaneously or contemporaneously) with one or more other agents, such as those useful in the treatment of diabetes or insulin resistance.

Anti-diabetic agents are generally categorized into six classes: biguanides; thiazolidinediones; sulfonylureas; inhibitors of carbohydrate absorption; fatty acid oxidase inhibitors and anti-lipolytic drugs; and weight-loss agents. Any of these agents can also be used in the methods disclosed herein. The anti-diabetic agents include those agents disclosed in Diabetes Care, 22(4):623-634. One class of anti-diabetic agents of use is the sulfonylureas, which are believed to increase secretion of insulin, decrease hepatic glucogenesis, and increase insulin receptor sensitivity. Another class of anti-diabetic agents of use the biguanide antihyperglycemics, which decrease hepatic glucose production and intestinal absorption, and increase peripheral glucose uptake and utilization, without inducing hyperinsulinemia.

In some examples, the FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) can be administered in combination with effective doses of anti-diabetic agents (such as biguanides, thiazolidinediones, or incretins) and/or lipid lowering compounds (such as statins or fibrates). The term “administration in combination” or “co-administration” refers to both concurrent and sequential administration of the active agents. Administration of the FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) or a nucleic acid encoding such a chimeric protein, may also be in combination with lifestyle modifications, such as increased physical activity, low fat diet, low sugar diet, and smoking cessation. Additional agents of use include, without limitation, anti-apoptotic substances such as the Nemo-Binding Domain and compounds that induce proliferation such as cyclin dependent kinase (CDK)-6, CDK-4 and Cyclin Dl. Other active agents can be utilized, such as antidiabetic agents for example, metformin, sulphonylureas (e.g., glibenclamide, tolbutamide, glimepiride), nateglinide, repaglinide, thiazolidinediones (e.g., rosiglitazone, pioglitazone), peroxisome proliferator-activated receptor (PPAR)-gamma-agonists (such as C1262570) and antagonists, PPAR-gamma/alpha modulators (such as KRP 297), alpha-glucosidase inhibitors (e.g., acarbose, voglibose), Dipeptidyl peptidase (DPP)-IV inhibitors (such as LAF237, MK-431), alpha2-antagonists, agents for lowering blood sugar, cholesterol-absorption inhibitors, 3-hydroxy-3-methylglutaryl-coenzyme A (HMGCoA) reductase inhibitors (such as a statin), insulin and insulin analogues, GLP-1 and GLP-1 analogues (e.g., exendin-4) or amylin. In some embodiments the agent is an immunomodulatory factor such as anti-CD3 mAb, growth factors such as HGF, vascular endothelial growth factor (VEGF), platelet derived growth factor (PDGF), lactogens, or parathyroid hormone related protein (PTHrP). In one example, the FGF2/FGF1 chimeric protein is administered in combination with a therapeutically effective amount of another FGF, such as FGF21, FGF19, or both, heparin, or combinations thereof.

In some embodiments, methods are provided for treating diabetes or pre-diabetes in a subject by administering a therapeutically effective amount of a composition including an FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or a nucleic acid encoding the chimeric protein, to the subject. The subject can have diabetes type I or diabetes type II. The subject can be any mammalian subject, including human subjects and veterinary subjects such as cats and dogs. The subject can be a child or an adult. The subject can also be administered insulin. The method can include measuring blood glucose levels.

In some examples, the method includes selecting a subject with diabetes, such as type I or type II diabetes, or a subject at risk for diabetes, such as a subject with pre-diabetes. These subjects can be selected for treatment with the disclosed FGF2/FGF1 chimeric proteins, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167) or nucleic acid molecules encoding such.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dL (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes Care 33:S11-61, 2010).

In some examples, the subject treated with the disclosed compositions and methods has HbA1C of greater than 6.5% or greater than 7%.

In some examples, treating diabetes includes one or more of increasing glucose tolerance, decreasing insulin resistance (for example, decreasing plasma glucose levels, decreasing plasma insulin levels, or a combination thereof), decreasing serum triglycerides, decreasing free fatty acid levels, and decreasing HbA1c levels in the subject. In some embodiments, the disclosed methods include measuring glucose tolerance, insulin resistance, plasma glucose levels, plasma insulin levels, serum triglycerides, free fatty acids, and/or HbA1c levels in a subject.

In some examples, administration of a FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or nucleic acid molecule encoding such, treats a metabolic disease, such as diabetes (such as type II diabetes) or pre-diabetes, by decreasing of HbA1C, such as a reduction of at least 0.5%, at least 1%, or at least 1.5%, such as a decrease of 0.5% to 0.8%, 0.5% to 1%, 1 to 1.5% or 0.5% to 2%. In some examples the target for HbA1C is less than about 6.5%, such as about 4-6%, 4-6.4%, or 4-6.2%. In some examples, such target levels are achieved within about 26 weeks, within about 40 weeks, or within about 52 weeks. Methods of measuring HbA1C are routine, and the disclosure is not limited to particular methods. Exemplary methods include HPLC, immunoassays, and boronate affinity chromatography.

In some examples, administration of a FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or nucleic acid molecule encoding such, treats diabetes or pre-diabetes by increasing glucose tolerance, for example, by decreasing blood glucose levels (such as two-hour plasma glucose in an OGTT or FPG) in a subject. In some examples, the method includes decreasing blood glucose by at least 5% (such as at least 10%, at least 15%, at least 20%, at least 25%, at least 30%, at least 35%, or more) as compared with a control (such as no administration of any of insulin, a FGF2/FGF1 chimeric protein, including those that further include a 3-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or a nucleic acid molecule encoding such). In particular examples, a decrease in blood glucose level is determined relative to the starting blood glucose level of the subject (for example, prior to treatment with a FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or nucleic acid molecule encoding such). In other examples, decreasing blood glucose levels of a subject includes reduction of blood glucose from a starting point (for example greater than about 126 mg/dL FPG or greater than about 200 mg/dL OGTT two-hour plasma glucose) to a target level (for example, FPG of less than 126 mg/dL or OGTT two-hour plasma glucose of less than 200 mg/dL). In some examples, a target FPG may be less than 100 mg/dL. In other examples, a target OGTT two-hour plasma glucose may be less than 140 mg/dL. Methods to measure blood glucose levels in a subject (for example, in a blood sample from a subject) are routine.

In other embodiments, the disclosed methods include comparing one or more indicator of diabetes (such as glucose tolerance, triglyceride levels, free fatty acid levels, or HbA1c levels) to a control (such as no administration of any of insulin, a FGF2/FGF1 chimeric protein, including those that further include a β-Klotho-binding peptide and/or FGFR1c-binding peptide, (such as one encoding a protein generated using the sequences shown in Table 1, 2 or 3, one or more sequences shown in SEQ ID NO: 22, 28, 29, 30, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175, or those encoding a protein having at least 90%, at least 92%, at least 95%, at least 98%, or at least 99% sequence identity to SEQ ID NO: 9, 10, 11, 12, 13, 99, 100, 101, 102, 103, 104, 156, 157, 158, 159, 160, 161, 162, 163, 164, 165, 166 or 167), or a nucleic acid molecule encoding such), wherein an increase or decrease in the particular indicator relative to the control (as discussed above) indicates effective treatment of diabetes. The control can be any suitable control against which to compare the indicator of diabetes in a subject. In some embodiments, the control is a sample obtained from a healthy subject (such as a subject without diabetes). In some embodiments, the control is a historical control or standard reference value or range of values (such as a previously tested control sample, such as a group of subjects with diabetes, or group of samples from subjects that do not have diabetes). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound in a cross-over study. In further examples, the control is the subject (or group of subjects) prior to treatment.

The disclosure is illustrated by the following non-limiting Examples.

### Example 1

### Preparation of Chimeric Proteins

FGF2/FGF1 chimeric proteins can be made using known methods (e.g., see Xia et al., PLoS One. 7(11):e48210, 2012).

Briefly, a nucleic acid sequence encoding a FGF2/FGF1 chimeric sequence (SEQ ID NO: 9) can be fused downstream of an enterokinase (EK) recognition sequence (Asp4Lys) preceded by a flexible 20 amino acid linker (derived from the S-tag sequence of pBAC-3) and an N-terminal (His)₆ tag. The resulting expressed fusion protein utilizes the (His)₆ tag for efficient purification and can be subsequently processed by EK digestion to yield the FGF2/FGF1 chimeric protein.

The FGF2/FGF1 chimeric protein can be expressed from an E. coli host after induction with isopropyl-β-D-thio-galactoside. The expressed protein can be purified utilizing sequential column chromatography on Ni-nitrilotriacetic acid (NTA) affinity resin followed by ToyoPearl HW-40S size exclusion chromatography. The purified protein can be digested with EK to remove the N-terminal (His)₆ tag, 20 amino acid linker, and (Asp₄Lys) EK recognition sequence. A subsequent second Ni-NTA chromatographic step can be utilized to remove the released N-terminal FGF2/FGF1 chimeric protein (along with any uncleaved fusion protein). Final purification can be performed using HiLoad Superdex 75 size exclusion chromatography equilibrated to 50 mM Na₂PO₄, 100 mM NaCl, 10 mM (NH₄)₂SO₄, 0.1 mM ethylenediaminetetraacetic acid (EDTA), 5 mM L-Methionine, pH at 6.5 (“PBX” buffer); L-Methionine can be included in PBX buffer to limit oxidization of reactive thiols and other potential oxidative degradation.

In some examples, the enterokinase is not used, and instead, an FGF2/FGF1 chimeric protein (such as one that includes an N-terminal methionine) can be made and purified using heparin affinity chromatography.

For storage and use, the purified chimeric protein can be sterile filtered through a 0.22 micron filter, purged with N2, snap frozen in dry ice and stored at −80° C. prior to use. The purity of the chimeric protein can be assessed by both Coomassie Brilliant Blue and Silver Stain Plus (BIO-RAD Laboratories, Inc., Hercules Calif.) stained sodium dodecylsulfate polyacrylamide gel electrophoresis (SDS PAGE). FGF2/FGF1 chimeric proteins can be prepared in the absence of heparin. Prior to IV bolus, heparin, or PBS, can be added to the protein.

### Example 2

### FGF1/FGF2 Chimera Reduces Blood Glucose in Ob/Ob Mice

Ob/ob mice about 6 month's in age were used. Mice were fed ad lib throughout the procedure. Blood glucose levels were measured from tail bleeds using a novaMax glucometer (Nova diabetes care, inc., USA).

Mice were injected subcutaneously at 0.5 mg/kg with (a) PBS; negative control; (b) mouse FGF1; positive control (SEQ ID NO: 8 but lacking first 15 a.a. MAEGEITTFAALTER and with an added M at N terminus); (c) FGF24 (SEQ ID NO: 9) or (d) FGF25 (SEQ ID NO: 35; human FGF1 (SEQ ID NO: 6) with codon usage changes to improve expression in bacteria).

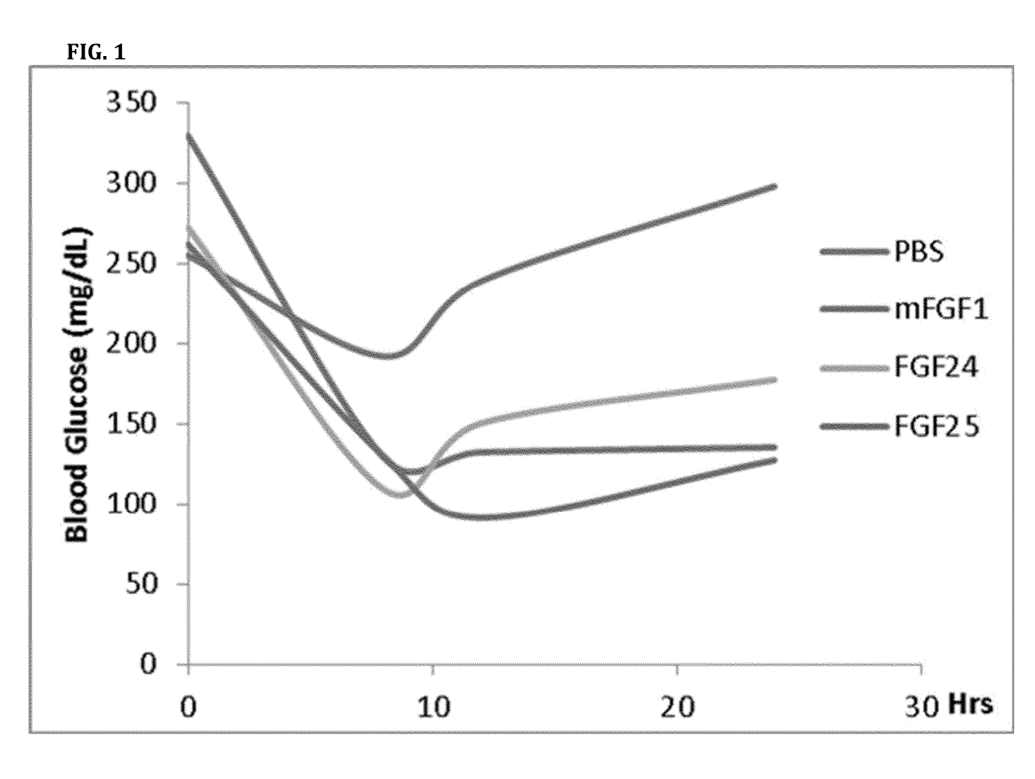

As shown in FIG. 1 and Tables 4 and 5 below, a single bolus of FGF24 and FGF25 can reduce blood glucose levels in obese and diabetic ob/ob mouse over a period of days.

TABLE 4
Glucose Trends

| | time | 3/12 2AM | 3/12 10AM | 3/12 2PM | 3/13 2AM |
|---|---|---|---|---|---|
| ID | treatment | 0 | 8 | 12 | 24 |
| 7-O | PBS | 245 | 191 | 236 | 226 |
| 7-R | PBS | 265 | 193 | 242 | 370 |
| 1-O | mFGF1 | 371 | 108 | 143 | 130 |
| 1-R | mFGF1 | 288 | 151 | 122 | 141 |
| 8-O | FGF24 | 270 | 113 | 131 | 225 |
| 8-R | FGF24 | 275 | 106 | 170 | 130 |
| 9-O | FGF25 | 315 | 126 | 88 | 158 |
| 9-R | FGF25 | 209 | 134 | 95 | 97 |

TABLE 5
Averages for each group (n = 2 per group)

| time | 3/12 2AM | 3/12 10AM | 3/12 2PM | 3/13 2AM |
|---|---|---|---|---|
| hrs post-inj | 0 | 8 | 12 | 24 |
| PBS | 255 | 192 | 239 | 298 |
| mFGF1 | 329.5 | 129.5 | 132.5 | 135.5 |
| FGF24 | 272.5 | 109.5 | 150.5 | 177.5 |
| FGF25 | 262 | 130 | 91.5 | 127.5 |

### Example 3

### Effect on Intracellular Signaling with FGF1 Mutants

Peptides M1, M2, M3, M4, and M5 (see SEQ ID NOS: 36, 42, 54, 68, and 19+a C117V mutation, respectively); KN (SEQ ID NO: 18), KLE (SEQ ID NO: 19); FGF1 (SEQ ID NO: 14) and NT2 (SEQ ID NO: 16) were generated as described in Example 1. The NT truncations, peptides NT1 (SEQ ID NO: 15), NT2 (SEQ ID NO: 16), and NT3 (SEQ ID NO: 17), were prepared without the His tag and enterokinase cleavage, and purified by heparin affinity. Peptides (10 ng/ml) were incubated with serum-starved HEK293 cells for 15 minutes. Total cell lysates were subject to Western blotting with antibodies specific for pAkt, Akt, pERK and ERK.

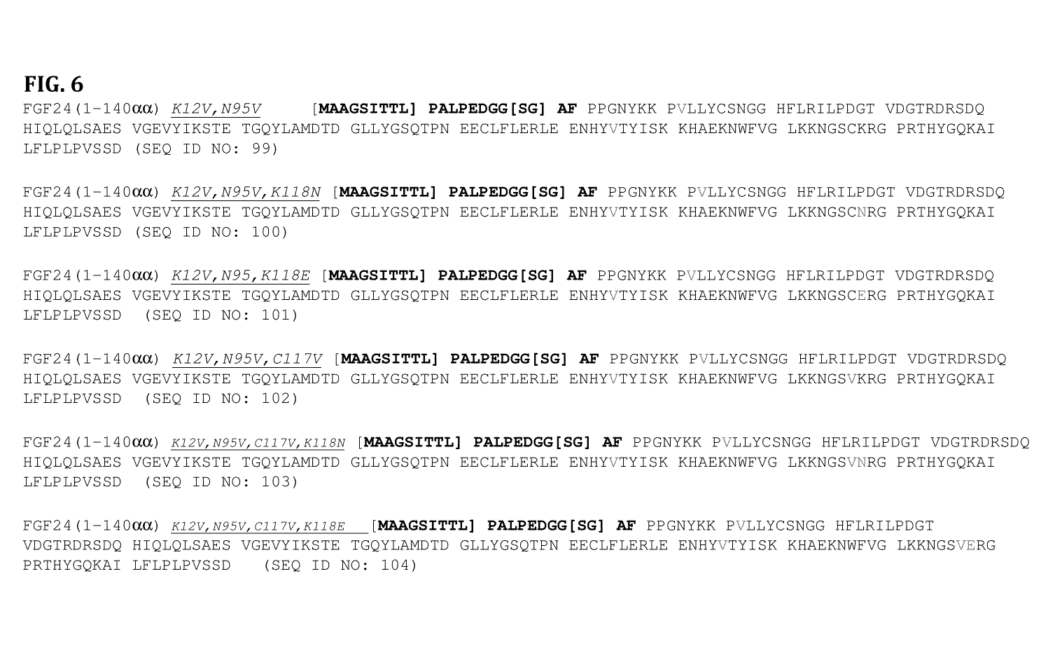

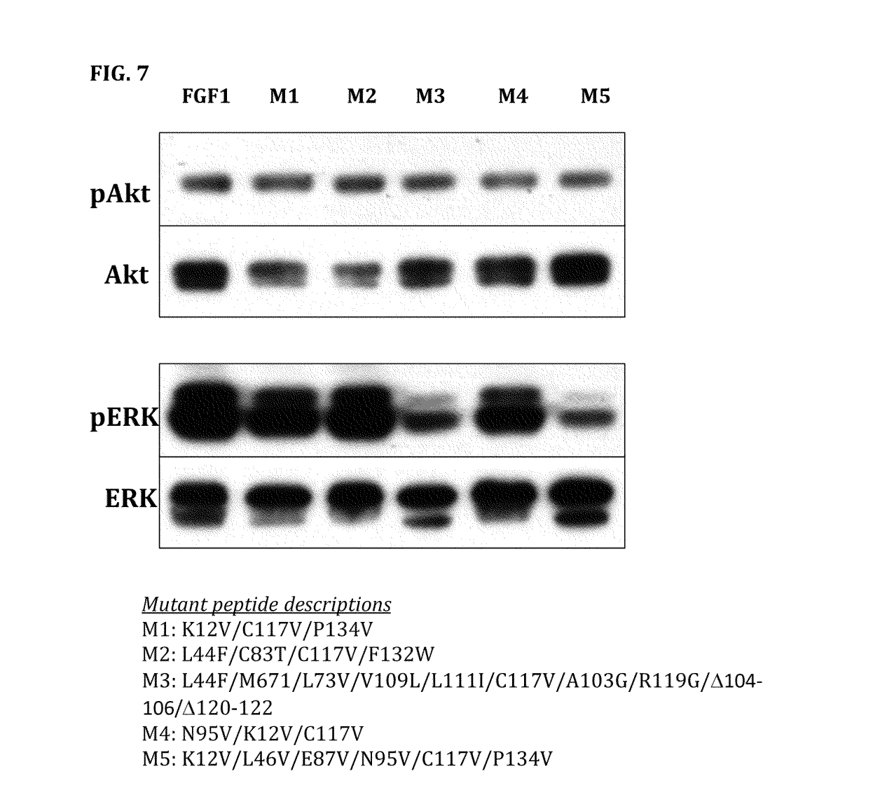

As shown in FIG. 7, the thermostable M3 analog shows reduced ERK signaling, similar to that seen with the M5 analog, correlating with the reduced glucose lowering activity seen in ob/ob mice.

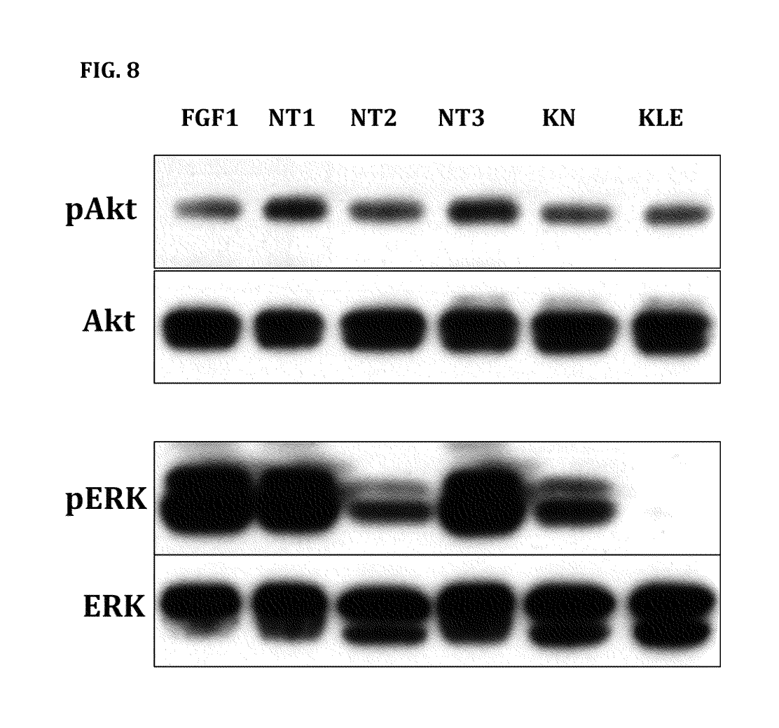

As shown in FIG. 8, deletion of 9 (NT1) or 11 (NT3)N-terminal residues of FGF1 does not significantly affect FGFR downstream signaling, while deletion of 13 (NT2) residues severely compromises ERK phosphorylation. The introduction of the point mutations K12V, N95V reduced ERK phosphorylation, while incorporating the additional mutations L46V, E87V and P134V totally abrogates ERK signaling.

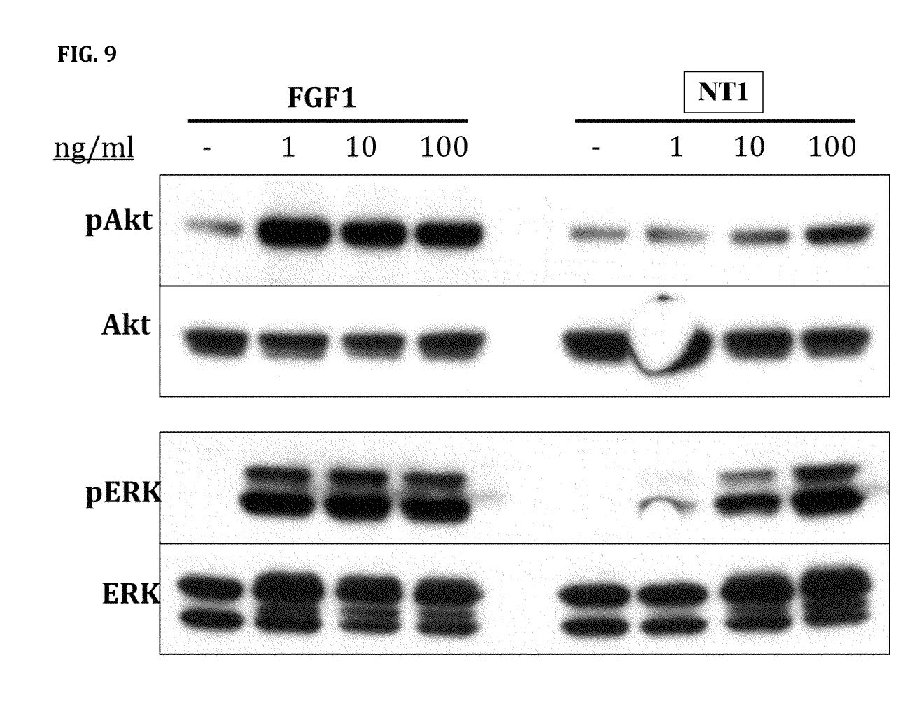

As shown in FIG. 9, deletion of 9 amino acids from the N-terminus of FGF1 (NT1, FGF1^{ΔNT}) induces an ˜100 fold reduction in FGFR signaling, as seen in the reduced phosphorylation of downstream ERK and AKT pathways.

### Example 4

### Effect on Blood Glucose with FGF1 Mutants

Peptides M1, M2, M3 (see SEQ ID NOS: 36, 42, and 54, respectively), FGF1 (SEQ ID NO: 14), NT1 (FGF1^{ΔNT}, SEQ ID NO: 15) and NT2 (FGF1^{ΔNT2}, SEQ ID NO: 16) were generated as described in Example 3. Peptides (0.5 mg/kg) or PBS (control) were injected SQ into 5 mo old C57BL/6J ob/ob mice fed normal chow. Blood glucose levels were subsequently determined.

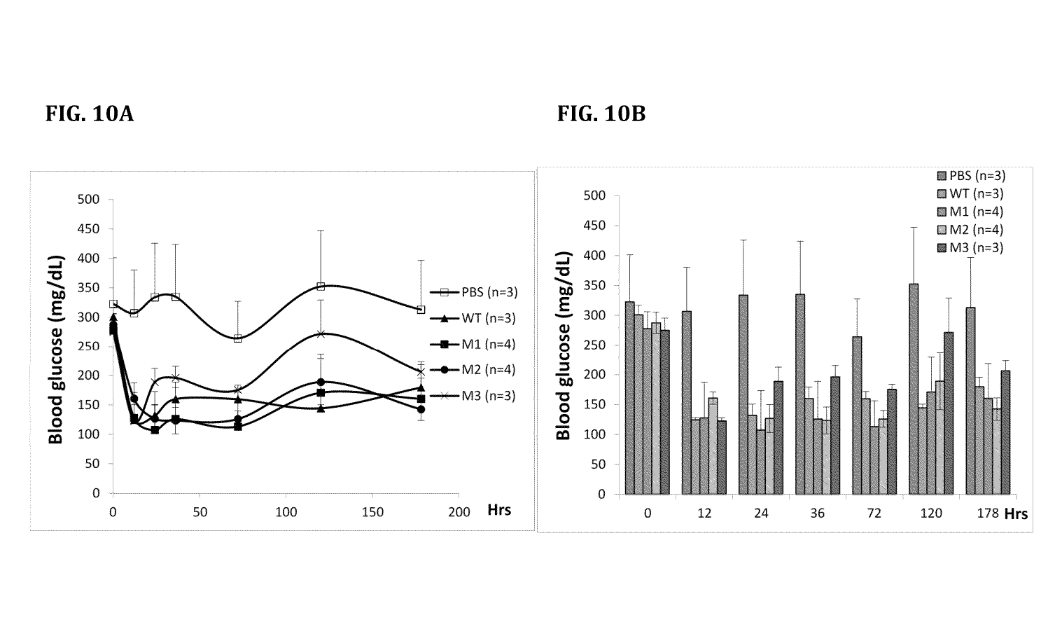

As shown in FIGS. 10A and 10B, peptides M1 and M2 lowered glucose as well as wild-type FGF1. Thus, FGF1 analogs can be designed with increased thermostability, and improved pharmacokinetic properties, while still having desired effects on lowing blood glucose. Thus, the FGF1 portion of the FGF2/FGF1 chimeras provided herein can include these mutations (e.g., one or more of K12V, C117V, P134V, L44F, C83T, and F132W).

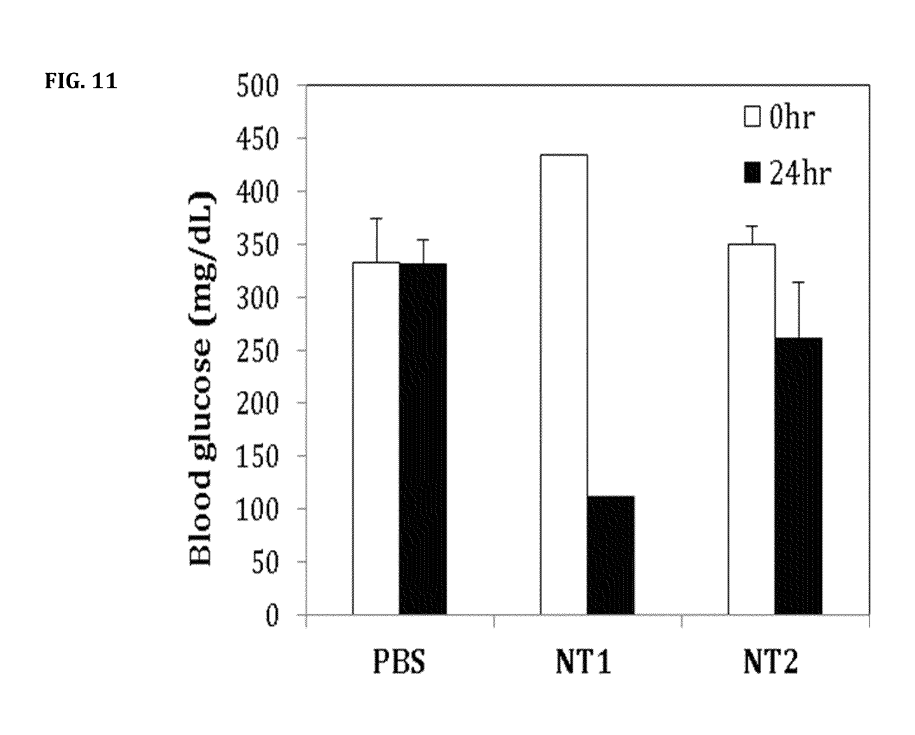

As shown in FIG. 11 peptide FGF1^{ΔNT} (NT1) significantly lowered glucose, while FGF1^{ΔNT2} (NT2) lost its ability to significantly lowered glucose. Thus, FGF1 can be N-terminally truncated (such as the first 9 amino acids, but not more than 13 amino acids), while still having desired effects on lowing blood glucose. Thus, the FGF1 portion of the FGF2/FGF1 chimeras provided herein can include such a truncation.

### Example 5

### Glucose Lowering Correlates with FGFR Signaling

Peptides FGF1 (SEQ ID NO: 14), NT1 (FGF1^{ΔNT}, SEQ ID NO: 15), and NT2 (SEQ ID NO: 16) were generated as described in Example 3. Peptides (10 ng/ml) were incubated with serum-starved HEK293 cells for 15 minutes. Total cell lysates were subject to Western blotting with antibodies specific for pAkt, Akt, pERK and ERK.

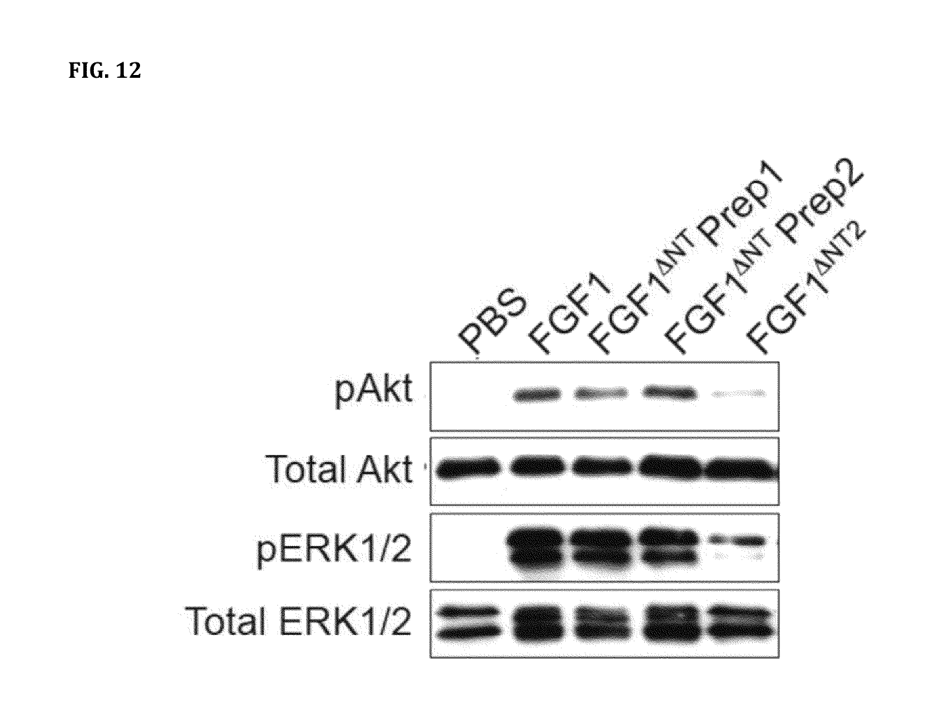

As shown in FIG. 12, comparable activation of the downstream signaling effectors ERK and AKT is seen with FGF1 and two independent preparations of FGF1^{ΔNT} that lacks the N-terminal 9 amino acids (SEQ ID NO: 15). In contrast, the deletion of an additional 4 N-terminal amino acids markedly reduces both ERK and AKT phosphorylation. These in vitro FGFR-mediated signaling results correlate with the in vivo glucose lowering effect observed in FIG. 11, supporting the hypothesis that the glucose-lowering activity is mediated through an FGF receptor.

### Example 6

### Effect on Blood Glucose with FGF1 Mutants

Peptides FGF1-KLE (SEQ ID NO: 19) and FGF1-KN (SEQ ID NO: 18) were generated as described in Example 1. Peptides (0.5 mg/kg) were injected SQ into 5 mo old C57BL/6J ob/ob mice fed normal chow. Blood glucose levels were subsequently determined 0 to 120 hours later.

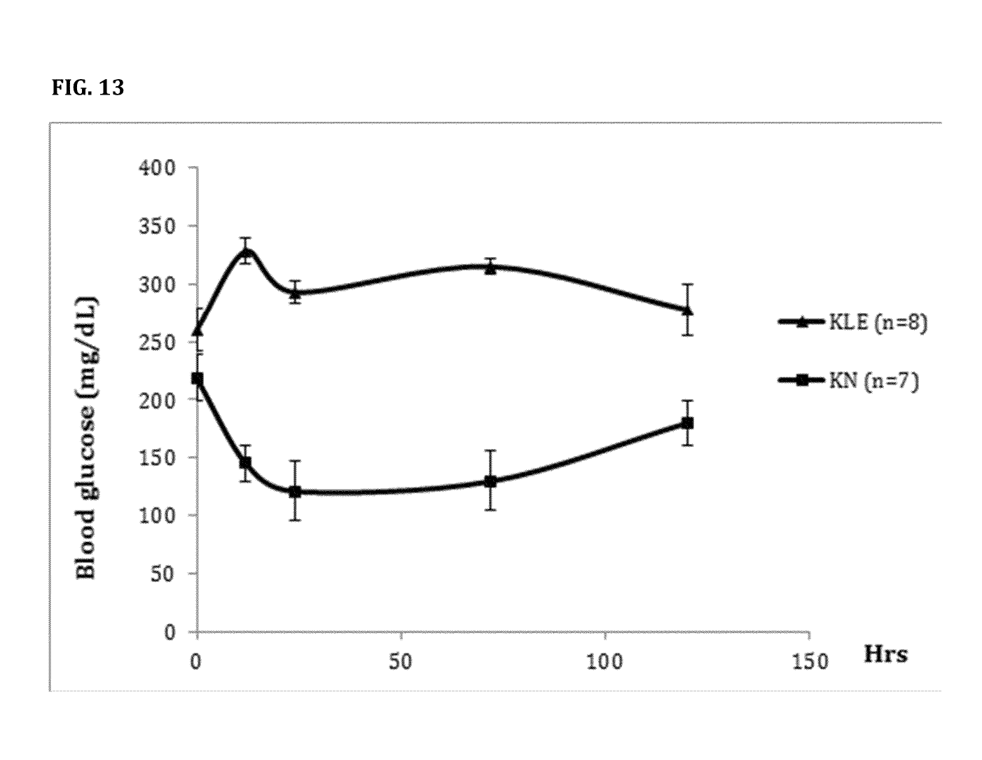

As shown in FIG. 13, the FGF1-KN mutant retained the ability to lower glucose for 120 hrs despite a marked reduction in its mitogenic activity. In contrast, the mitogenically dead FGF1-KLE failed to lower glucose. These results indicate that the mitogenicity and glucose-lowering activity can be independently affected through targeted mutations. Thus, the FGF1 portion of the FGF2/FGF1 chimeras provided herein can include the mutations in the KN mutant (e.g., one or more of K12V and N95V) to reduce its mitogenicity without significantly compromising its ability to lower blood glucose levels.

### Example 7

Dose-Response Effects on Blood Glucose with FGF1 Mutants Peptides rFGF1^{ΔNT} (SEQ ID NO: 15), or rFGF1 (SEQ ID NO: 14) were generated as described in Example 3 (generated with an N-terminal methionine and purified with heparin affinity and ion exchange chromatography). Peptides (0.016 to 10 ng/ml) or PBS were incubated with serum-starved HEK293 cells for 15 minutes. Total cell lysates were subject to Western blotting with antibodies specific for pFRS2a, pAkt, Akt, pERK and ERK. Peptides (0.016 mg/kg) were injected SQ into diabetes-induced model (DIO) 5 mo old C57BL/6J ob/ob mice fed normal chow, or into peptides (0 to 0.5 mg/kg) were injected SQ into high fat diet (HFD) fed diet-induced obesity (DIO) mice or into 12 week old C57BL/6J ob/ob mice (0 to 0.5 mg/kg) fed normal chow. Blood glucose levels were subsequently determined.

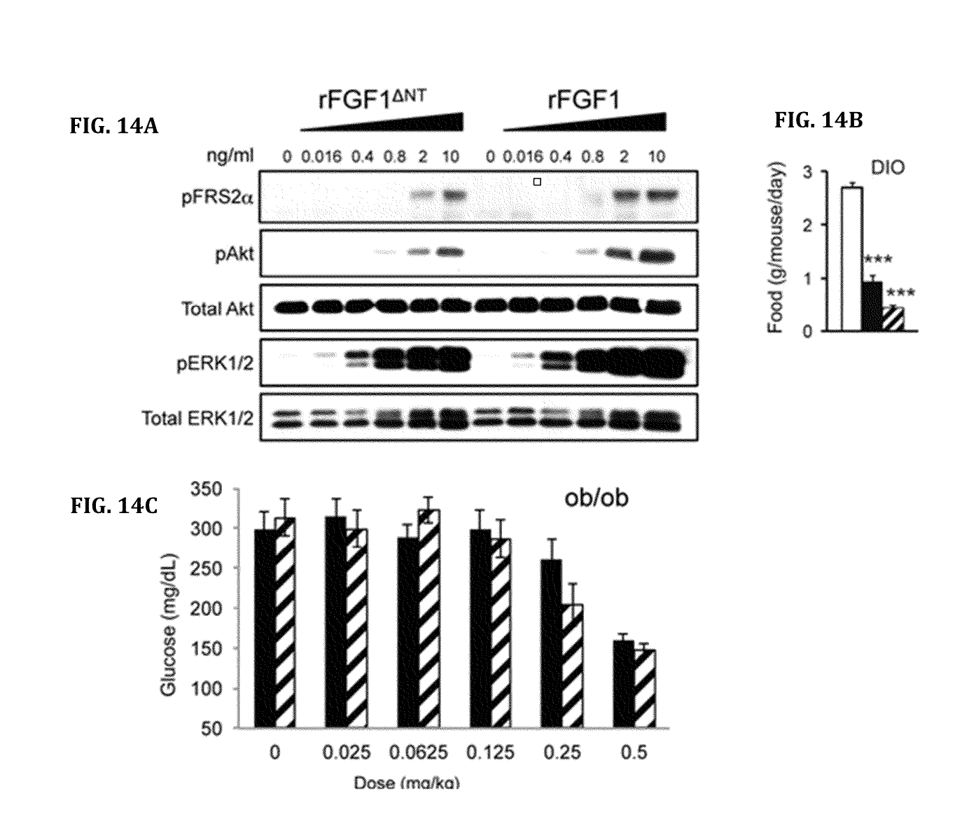

As shown in FIG. 14A, deletion of 9 N-terminal amino acids of FGF1 significantly reduces FGFR downstream signaling, including phosphorylation of ERK and AKT. Dose dependent phosphorylation of the FGFR substrate FRS2a, confirms that both FGF1 and FGF1^{ΔNT} are capable of activating FGF receptors.

As shown in FIG. 14B, food intake in DIO mice after receiving, rFGF1 or rFGF1^{ΔNT} was significantly reduced, as compared to mice that received PBS alone. The similarity in the extent of the transient reduction in food intake between rFGF1 and rFGF1^{ΔNT} further supports the conclusion that both proteins achieve their in vivo glucose lowering effects by signaling through an FGF receptor.

As shown in FIG. 14C, an essentially identical dose-response curve was observed for the glucose lowering effects of rFGF1 and rFGF1^{ΔNT} (NT1) in ob/ob mice. Given the significant reduction in mitogenicity of rFGF1^{ΔNT}, these results demonstrate that the glucose lowering and mitogenic activities of FGF1 can be dissociated.

### Example 8

Effect of N-terminal FGF1 Truncations on Blood Glucose Levels Peptides NT1 (SEQ ID NO: 15), NT2 (SEQ ID NO: 16), or NT3 (SEQ ID NO: 17) were generated as described in Example 3. Peptides (0.5 mg/kg) were injected SQ into 5 mo old C57BL/6J ob/ob mice fed normal chow, or peptides (0 to 0.5 mg/kg) were injected SQ into 12 week old ob/ob mice fed normal chow. Blood glucose levels were subsequently determined (0 hr, 16 hrs, or 24 hrs).

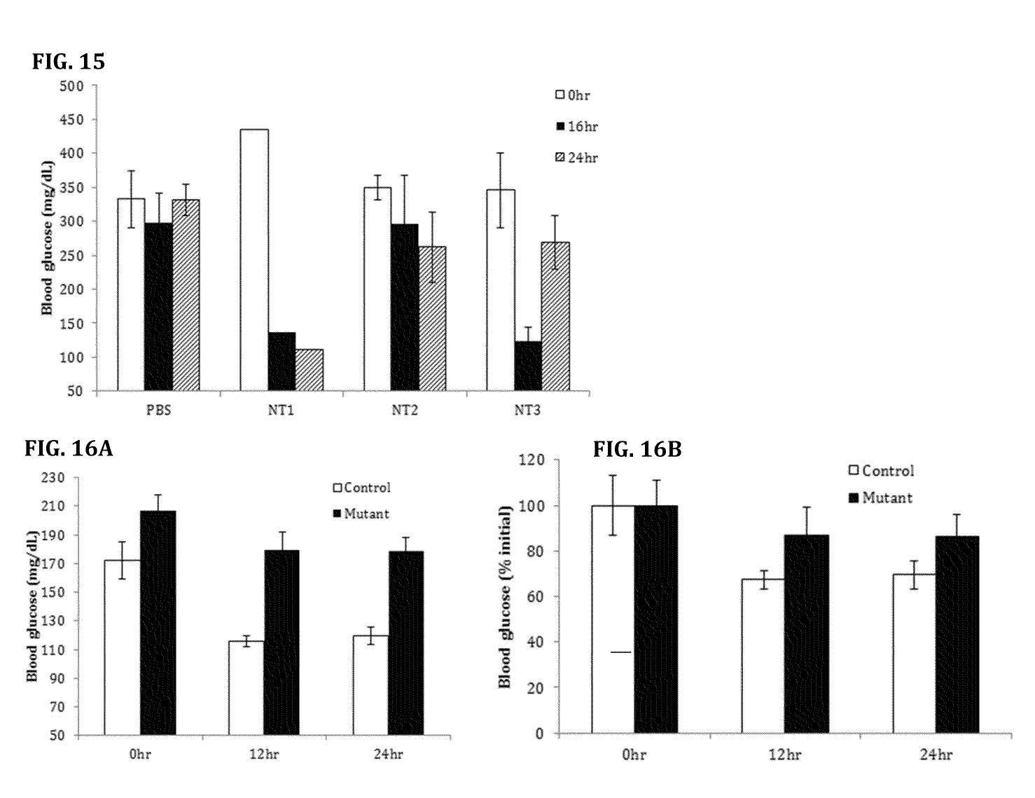

As shown in FIG. 15 if the N-terminus is truncated at 14 amino acids, glucose lowering ability is dramatically decreased (NT2). Thus, FGF1 can be N-terminally truncated (such as the first 9, 10, or 11 amino acids), while maintaining the desired effects on lowering blood glucose. Thus, the FGF1 portion of the FGF2/FGF1 chimeras provided herein can include such a truncation.

In another experiment, NT1 (SEQ ID NO: 15) (0.5 mg/kg) was injected SQ into 8 month old HFD-fed wildtype (FGFR1 f/f, open bars) or adipose-specific FGFR1 knockout (R1 KO, aP2-Cre; FGFR1 f/f, filled bars) mice Blood glucose levels were subsequently determined (0 hr, 12 hrs, or 24 hrs).

As shown in FIGS. 16A and 16B, rFGF1^{ΔNT} (NT1) (SEQ ID NO: 15) lowers blood glucose levels in HFD-fed wildtype mice (control) but has no effect on FGFR1 KO (mutant) mice. FIG. 16A reports the changes in blood glucose, while FIG. 16B reports the data normalized to starting glucose levels at 100%. These results demonstrate that expression of FGFR1 in adipose tissue is required for rFGF1^{ΔNT} mediated glucose lowering.

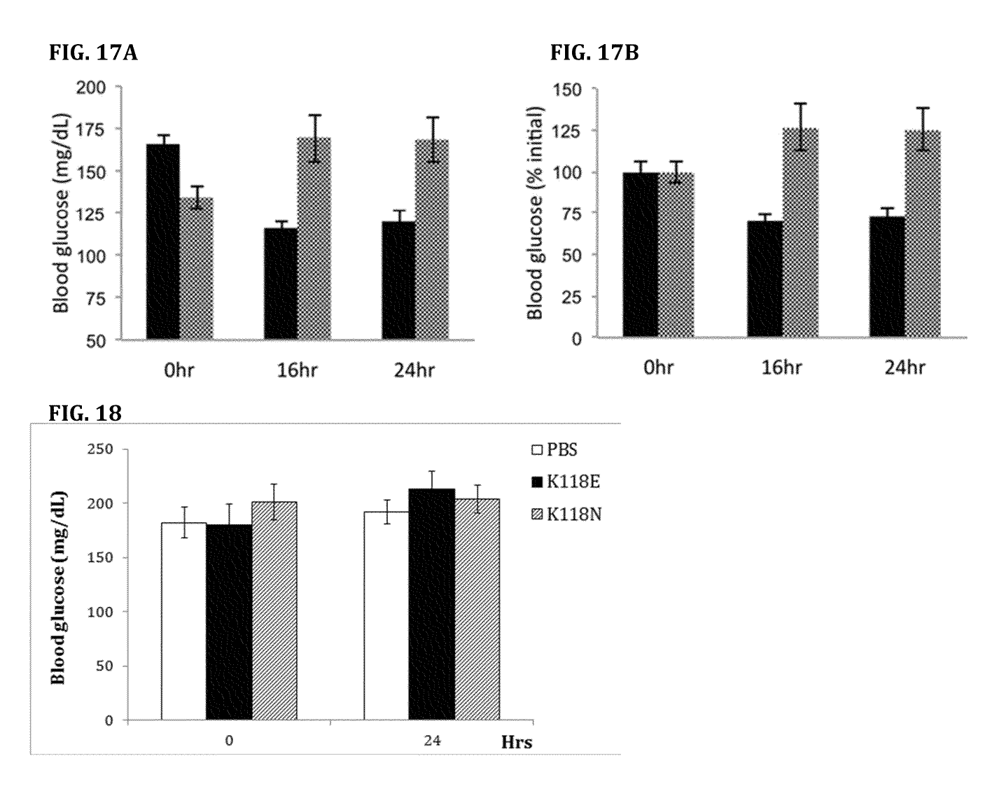

As shown in FIGS. 17A and 17B mouse rFGF1 (amino acids 15-155 of SEQ ID NO: 8) lowers blood glucose levels in HFD-fed wildtype mice (FGFR1 f/f mice, filled bars) but has no effect on aP2-Cre; FGFR1 f/f (FGFR1 KO, speckled bars) mice. FIG. 17A reports the changes in blood glucose, while FIG. 17B reports the data normalized to starting glucose levels at 100%. These results demonstrate that expression of FGFR1 in adipose tissue is required for rFGF1 mediated glucose lowering.

### Example 9

### Effect of FGF1 Point Mutations on Blood Glucose Lowering

Peptides K118E (SEQ ID NO: 21), K118N (SEQ ID NO: 20), FGF1 (SEQ ID NO: 14), and KKK (SEQ ID NO: 168) were generated as described in Example 1, while FGF1^{ΔNT} (NT1) (SEQ ID NO: 15) was expressed with an N-terminal methionine and purified using heparin affinity and ion exchange chromatography. Peptides (0.5 mg/kg) or PBS were injected SQ into 7 months HFD-fed C57BL/6J mice. Blood glucose levels were subsequently determined (0 hr or 24 hrs). These mice are diet-induced obese (DIO) mice.

As shown in FIG. 18, mutation of the single lysine, K118, to either Asn (K118N, SEQ ID NO: 20) or Glu (K118E, SEQ ID NO: 21), is sufficient to abrogate glucose lowering activity in DIO mice.

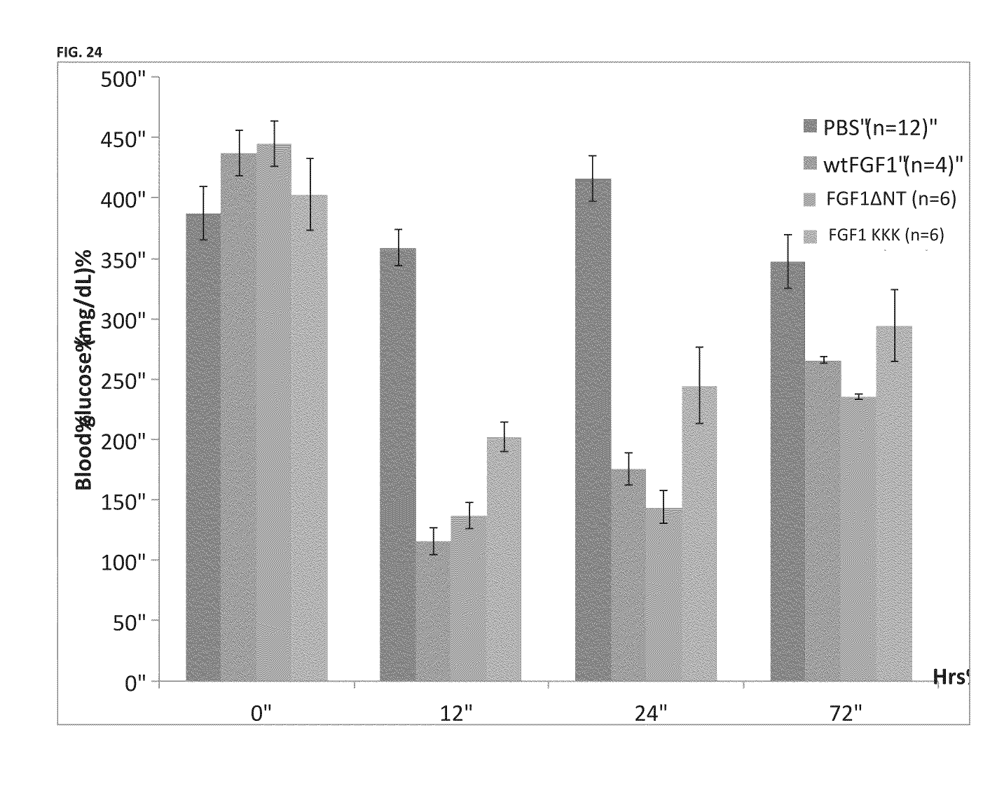

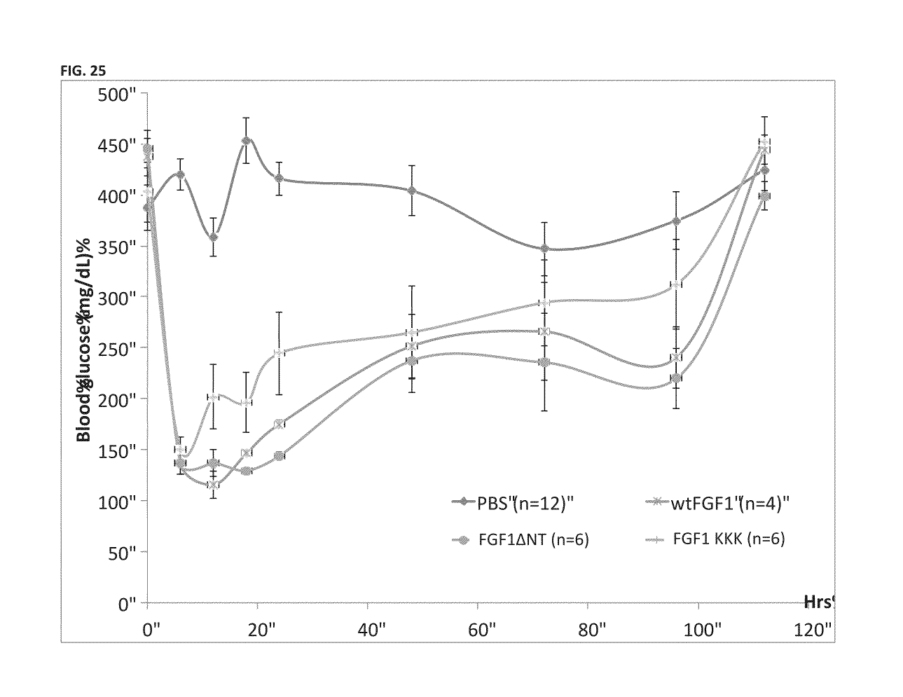

As shown in FIGS. 24 and 25, mutating selected amino acids implicated in the heparin binding site of FGF1, namely amino acids K112, K113, and K118, resulted in a mutated FGF1 sequence that could lower blood glucose levels in ob/ob mice. Thus, the FGF/2FGF1 chimeras provided herein can include mutations in the FGF1 portion at all three of K112, K113, and K118, such as a K112D, K113Q, and K118V substitution. However, while the mutation of K118 to the hydrophobic residue valine was tolerated, mutations involving a charge reversal (K118E) or to a polar residue (K118N) are not tolerated.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. A chimeric protein comprising:
an N-terminus coupled to a C-terminus, wherein the N-terminus comprises an N-terminal portion of fibroblast growth factor (FGF) 2 protein and the C-terminus comprises a portion of an FGF1 protein.

2. The chimeric protein of claim 1, wherein the N-terminus comprises at least 12 consecutive amino acids from amino acids 1-30 of FGF2 and the C-terminus comprises at least 120 consecutive amino acids from amino acids 5-141 of FGF1, wherein the at least 12 consecutive amino acids from amino acids 1-30 of FGF2 can comprise 1, 2, 3 or 4 point mutations, and wherein the at least 120 consecutive amino acids from amino acids 5-141 of FGF1 can comprise 1-20 point mutations.

3. The chimeric protein of claim 2, wherein the at least 12 consecutive amino acids from amino acids 1-30 of FGF2 comprise or consist of:
MAAGSITTLP ALPEDGGSGA F (amino acids 1 to 21 of SEQ ID NO: 2),
MAASGITSLP ALPEDGGAAF (amino acids 1 to 20 of SEQ ID NO: 4),
PALPEDGGSGAF (amino acids 10 to 21 of SEQ ID NO: 2), or
PALPEDGGAAF (amino acids 10 to 20 of SEQ ID NO: 4).

4. The chimeric protein of claim 2, wherein the at least 12 consecutive amino acids from amino acids 1-30 of FGF2 comprising 1, 2, 3 or 4 point mutations comprise or consist of:
MAAGSITTLP ALPEDGGSFA F (amino acids 1 to 21 of SEQ ID NO: 10);
MAAGSITTLP ALPEDGGSFNL (amino acids 1 to 21 of SEQ ID NO: 11);
PALPEDGGSFAF (amino acids 10 to 21 of SEQ ID NO: 10);
PALPEDGGSFNL (amino acids 10 to 21 of SEQ ID NO: 11);
MPALPEDGGSGAF (amino acids 1 to 13 of SEQ ID NO: 13);
MPALPEDGGAAF (amino acids 1 to 12 of SEQ ID NO: 28);
MPALPEDGFAAF (amino acids 1 to 12 of SEQ ID NO: 29); or
MPALPEDGFFSGAF (amino acids 1 to 14 of SEQ ID NO: 30).

5. The chimeric protein of claim 2, wherein the at least at least 120 consecutive amino acids of FGF1 comprise or consist of:
(amino acids 4 to 140 of SEQ ID NO: 14)
PPGNYK KPKLLYCSNG GHFLRILPDG TVDGTRDRSD QHIQLQLSAE
SVGEVYIKST ETGQYLAMDT DGLLYGSQTP NEECLFLERL
EENHYNTYIS KKHAEKNWFVGLKKNGSCKR GPRTHYGQKA
ILFLPLPVSSD;
the protein sequence shown in SEQ ID NO: 14;
the protein sequence shown in SEQ ID NO: 15;
the protein sequence shown in SEQ ID NO: 16;
the protein sequence shown in SEQ ID NO: 17; or
the protein sequence shown in SEQ ID NO: 27.

6. The chimeric protein of claim 2, wherein the at least at least 120 consecutive amino acids from amino acids 5-141 of FGF1 comprising 1-20 point mutations comprise or consist of:
the protein sequence shown in SEQ ID NO: 18;
the protein sequence shown in SEQ ID NO: 19;
the protein sequence shown in SEQ ID NO: 20;
the protein sequence shown in SEQ ID NO: 21; or
the protein sequence shown in SEQ ID NO: 37, 38, 39, 40, 41, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 95, 97, 98, 168, 169, 170, 171, 172, 173, 174, or 175.

7. The chimeric protein of claim 1, wherein the N-terminal amino acid is a methionine.

8. The chimeric protein of claim 1, wherein the portion of the FGF1 protein is modified to decrease binding affinity for heparin and/or heparan sulfate compared to the portion of the FGF1 protein without the modification.

9. The chimeric protein of claim 1, further comprising:
one or more β-Klotho-binding proteins at the N-terminus or the C-terminus;
one or more fibroblast growth factor receptor (FGFR) 1c-binding proteins at the N-terminus or the C-terminus;
one or more β-Klotho-binding proteins and one or more fibroblast growth factor receptor (FGFR) 1c-binding proteins at the N-terminus or the C-terminus; or
one or more β-Klotho-binding proteins and one or more fibroblast growth factor receptor (FGFR) 1c-binding proteins at the N-terminus and the C-terminus.

10. The chimeric protein of claim 1, wherein the chimeric protein is
130-160 amino acids in length;
200 to 400 amino acids in length;
300 to 375 amino acids in length; or
250 to 300 amino acids in length.

11. The chimeric protein of claim 1, wherein the chimeric protein comprises at least 80%, at least 85%, at least 90%, at least 95%, at least 96%, at least 97%, at least 98% or at least 99% sequence identity to any of SEQ ID NOS: 9, 10, 11, 12, 13, 99, 100, 101, 102, 103, 104, 156, 157, 158, 159, 160, 161, 162, 163, 164, 165, 166 and 167.

12. An isolated nucleic acid encoding the chimeric protein of claim 1.

13. A nucleic acid vector comprising the isolated nucleic acid of claim 12.

14. A host cell comprising the vector of claim 13.

15. The host cell of claim 13, wherein the host cell is a bacterium or yeast cell.

16. The host cell of claim 15, wherein the bacterium is E. coli.

17. A method of reducing blood glucose in a mammal, comprising:
administering a therapeutically effective amount of the protein of claim 1 to the mammal, thereby reducing the blood glucose.

18. A method of treating a metabolic disease in a mammal, comprising:
administering a therapeutically effective amount of the protein of claim 1 to the mammal, thereby treating the metabolic disease.

19. The method of claim 18, wherein the metabolic disease is type 2 diabetes, non-type 2 diabetes, type 1 diabetes, polycystic ovary syndrome (PCOS), metabolic syndrome (MetS), obesity, non-alcoholic steatohepatitis (NASH), non-alcoholic fatty liver disease (NAFLD), hyperlipidemia, hypertension, latent autoimmune diabetes (LAD), or maturity onset diabetes of the young (MODY).

20. A method of reducing fed and fasting blood glucose, improving insulin sensitivity and glucose tolerance, reducing systemic chronic inflammation, ameliorating hepatic steatosis in a mammal, or combinations thereof, comprising:
administering a therapeutically effective amount of the protein of claim 1 to the mammal, thereby reducing fed and fasting blood glucose, improving insulin sensitivity and glucose tolerance, reducing systemic chronic inflammation, and ameliorating hepatic steatosis in a mammal, or combinations thereof.

21. The method of claim 17, wherein the therapeutically effective amount of the protein is at least 0.5 mg/kg.

22. The method of claim 17, wherein the administering is subcutaneous, intraperitoneal, intramuscular, or intravenous.

23. The method of claim 17, wherein the mammal is a cat or dog.

24. The method of claim 17, wherein the mammal is a human.

25. The method of claim 17, wherein the method further comprises administering an additional therapeutic compound.

26. The method of claim 25, wherein the additional therapeutic compound is one or more of an alpha-glucosidase inhibitor, amylin agonist, dipeptidyl-peptidase 4 (DPP-4) inhibitor, meglitinide, sulfonylurea, or a peroxisome proliferator-activated receptor (PPAR)-gamma agonist.

27. The method of claim 26, wherein the PPAR-gamma agonist is a thiazolidinedione (TZD), aleglitazar, farglitazar, muraglitazar, or tesaglitazar.

28. The method of claim 27, wherein the TZD is pioglitazone, rosiglitazone, rivoglitazone, or troglitazone.
